SEQUENCE LISTING (20 sequences)

SEQ ID NO: 1 - DNA, 7070 bp, Artificial Sequence
  Description of Artificial Sequence Synthetic nucleotide sequence
tcgagaggcctgacgtcgggcccggtaccacgcgtcatatgactagttggagaatcatgacctcagcatctgccccaagctttaaccccggcaagggtcccggctcagcagtcggaattgcccttttaggattcggaacagtcggcactgaggtgatgcgtctgatgaccgagtacggtgatgaacttgcgcaccgcattggtggcccactggaggttcgtggcattgctgtttctgatatctcaaagccacgtgaaggcgttgcacctgagctgctcactgaggacgcttttgcactcatcgagcgcgaggatgttgacatcgtcgttgaggttatcggcggcattgagtacccacgtgaggtagttctcgcagctctgaaggccggcaagtctgttgttaccgccaataaggctcttgttgcagctcactctgctgagcttgctgatgcagcggaagccgcaaacgttgacctgtacttcgaggctgctgttgcaggcgcaattccagtggttggcccactgcgtcgctccctggctggcgatcagatccagtctgtgatgggcatcgttaacggcaccaccaacttcatcttggacgccatggattccaccggcgctgactatgcagattctttggctgaggcaactcgtttgggttacgccgaagctgatccaactgcagacgtcgaaggccatgacgccgcatccaaggctgcaattttggcatccatcgctttccacacccgtgttaccgcggatgatgtgtactgcgaaggtatcagcaacatcagcgctgccgacattgaggcagcacagcaggcaggccacaccatcaagttgttggccatctgtgagaagttcaccaacaaggaaggaaagtcggctatttctgctcgcgtgcacccgactctattacctgtgtcccacccactggcgtcggtaaacaagtcctttaatgcaatctttgttgaagcagaagcagctggtcgcctgatgttctacggaaacggtgcaggtggcgcgccaaccgcgtctgctgtgcttggcgacgtcgttggtgccgcacgaaacaaggtgcacggtggccgtgctccaggtgagtccacctacgctaacctgccgatcgctgatttcggtgagaccaccactcgttaccacctcgacatggatgtggaagatcgcgtgggggttttggctgaattggctagcctgttctctgagcaaggaatcttcctgcgtacaatccgacaggaagagcgcgatgatgatgcacgtctgatcgtggtcacccactctgcgctggaatctgatctttcccgcaccgttgaactgctgaaggctaagcctgttgttaaggcaatcaacagtgtgatccgcctcgaaagggactaattttactgacatggcaattgaactgaacgtcggtcgtaaggttaccgtcacggtacctggatcttctgcaaacctcggacctggctttgacactttaggtttggcactgtcggtatacgacactgtcgaagtggaaattattccatctggcttggaagtggaagtttttggcgaaggccaaggcgaagtccctcttgatggctcccacctggtggttaaagctattcgtgctggcctgaaggcagctgacgctgaagttcctggattgcgagtggtgtgccacaacaacattccgcagtctcgtggtcttggctcctctgctgcagcggcggttgctggtgttgctgcagctaatggtttggcggatttcccgctgactcaagagcagattgttcagttgtcctctgcctttgaaggccacccagataatgctgcggcttctgtgctgggtggagcagtggtgtcgtggacaaatctgtctatcgacggcaagagccagccacagtatgctgctgtaccacttgaggtgcaggacaatattcgtgcgactgcgctggttcctaatttccacgcatccaccgaagctgtgcgccgagtccttcccactgaagtcactcacatcgatgcgcgatttaacgtgtcccgcgttgcagtgatgatcgttgcgttgcagcagcgtcctgatttgctgtgggagggtactcgtgaccgtctgcaccagccttatcgtgcagaagtgttgcctattacctctgagtgggtaaaccgcctgcgcaaccgtggctacgcggcatacctttccggtgccggcccaaccgccatggtgctgtccactgagccaattccagacaaggttttggaagatgctcgtgagtctggcattaaggtgcttgagcttgaggttgcgggaccagtcaaggttgaagttaaccaaccttaggcccaacaaggaaggcccccttcgaatcaagaagggggccttattagtgcagcaattattcgctgaacacgtgaaccttacaggtgcccggcgcgttgagtggtttgagttccagctggatgcggttgttttcaccgaggctttcttggatgaatccggcgtggatggcgcagacgaaggctgatgggcgtttgtcgttgaccacaaatgggcagctgtgtagagcgagggagtttgcttcttcggtttcggtggggtcaaagcccatttcgcggaggcggttaatgagcggggagagggcttcgtcgagttcttcggcttcggcgtggttaatgcccatgacgtgtgcccactgggttccgatggaaagtgctttggcgcggaggtcggggttgtgcattgcgtcatcgtcgacatcgccgagcatgttggccatgagttcgatcagggtgatgtattctttggcgacagcgcggttgtcggggacgcgtgtttggaagatgagggaggggcgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 2 - DNA, 7070 bp, Artificial Sequence; identical to SEQ ID NO: 1
  Description of Artificial Sequence Synthetic nucleotide sequence

SEQ ID NO: 3 - DNA, 8766 bp, Artificial Sequence
  Description of Artificial Sequence Synthetic nucleotide sequence
tcgagaggcctgacgtcgggcccggtaccacgcgtcatatgactagttcggacctagggatatcgtcgacatcgatgctcttctgcgttaattaacaattgggatctctcaactaatgcagcgatgcgttctttccagaatgctttcatgacagggatgctgtcttgatcaggcaggcgtctgtgctggatgccgaagctggatttattgtcgcctttggaggtgaagttgacgctcactcgagaatcatcggccaaccatttggcattgaatgttctaggttcggaggcggaggttttctcaattagtgcgggatcgagccactgcgcccgcaggtcatcgtctccgaagagcttccacactttttcgaccggcaggttaagggttttggaggcattggccgcgaacccatcgctggtcatcccgggtttgcgcatgccacgttcgtattcataaccaatcgcgatgccttgagcccaccagccactgacatcaaagttgtccacgatgtgctttgcgatgtgggtgtgagtccaagaggtggcttttacgtcgtcaagcaattttagccactcttcccacggctttccggtgccgttgaggatagcttcaggggacatgcctggtgttgagccttgcggagtggagtcagtcatgcgaccgagactagtggcgctttgggtaccgggccccccctcgaggtcgagcggcttaaagtttggctgccatgtgaatttttagcaccctcaacagttgagtgctggcactctcgggggtagagtgccaaataggttgtttgacacacagttgttcacccgcgacgacggctgtgctggaaacccacaaccggcacacacaaaatttttctcatggagggattcatcatgtcgacttcagttacttcaccagcccacaacaacgcacattcctccgaatttttggatgcgttggcaaaccatgtgttgatcggcgacggcgccatgggcacccagctccaaggctttgacctggacgtggaaaaggatttccttgatctggaggggtgtaatgagattctcaacgacacccgccctgatgtgttgaggcagattcaccgcgcctactttgaggcgggagctgacttggttgagaccaatacttttggttgcaacctgccgaacttggcggattatgacatcgctgatcgttgccgtgagcttgcctacaagggcactgcagtggctagggaagtggctgatgagatggggccgggccgaaacggcatgcggcgtttcgtggttggttccctgggacctggaacgaagcttccatcgctgggccatgcaccgtatgcagatttgcgtgggcactacaaggaagcagcgcttggcatcatcgacggtggtggcgatgcctttttgattgagactgctcaggacttgcttcaggtcaaggctgcggttcacggcgttcaagatgccatggctgaacttgatacattcttgcccattatttgccacgtcaccgtagagaccaccggcaccatgctcatgggttctgagatcggtgccgcgttgacagcgctgcagccactgggtatcgacatgattggtctgaactgcgccaccggcccagatgagatgagcgagcacctgcgttacctgtccaagcacgccgatattcctgtgtcggtgatgcctaacgcaggtcttcctgtcctgggtaaaaacggtgcagaatacccacttgaggctgaggatttggcgcaggcgctggctggattcgtctccgaatatggcctgtccatggtgggtggttgttgtggcaccacacctgagcacatccgtgcggtccgcgatgcggtggttggtgttccagagcaggaaacctccacactgaccaagatccctgcaggccctgttgagcaggcctcccgcgaggtggagaaagaggactccgtcgcgtcgctgtacacctcggtgccattgtcccaggaaaccggcatttccatgatcggtgagcgcaccaactccaacggttccaaggcattccgtgaggcaatgctgtctggcgattgggaaaagtgtgtggatattgccaagcagcaaacccgcgatggtgcacacatgctggatctttgtgtggattacgtgggacgagacggcaccgccgatatggcgaccttggcagcacttcttgctaccagctccactttgccaatcatgattgactccaccgagccagaggttattcgcacaggccttgagcacttgggtggacgaagcatcgttaactccgtcaactttgaagacggcgatggccctgagtcccgctaccagcgcatcatgaaactggtaaagcagcacggtgcggccgtggttgcgctgaccattgatgaggaaggccaggcacgtaccgctgagcacaaggtgcgcattgctaaacgactgattgacgatatcaccggcagctacggcctggatatcaaagacatcgttgtggactgcctgaccttcccgatctctactggccaggaagaaaccaggcgagatggcattgaaaccatcgaagccatccgcgagctgaagaagctctacccagaaatccacaccaccctgggtctgtccaatatttccttcggcctgaaccctgctgcacgccaggttcttaactctgtgttcctcaatgagtgcattgaggctggtctggactctgcgattgcgcacagctccaagattttgccgatgaaccgcattgatgatcgccagcgcgaagtggcgttggatatggtctatgatcgccgcaccgaggattacgatccgctgcaggaattcatgcagctgtttgagggcgtttctgctgccgatgccaaggatgctcgcgctgaacagctggccgctatgcctttgtttgagcgtttggcacagcgcatcatcgacggcgataagaatggccttgaggatgatctggaagcaggcatgaaggagaagtctcctattgcgatcatcaacgaggaccttctcaacggcatgaagaccgtgggtgagctgtttggttccggacagatgcagctgccattcgtgctgcaatcggcagaaaccatgaaaactgcggtggcctatttggaaccgttcatggaagaggaagcagaagctaccggatctgcgcaggcagagggcaagggcaaaatcgtcgtggccaccgtcaagggtgacgtgcacgatatcggcaagaacttggtggacatcattttgtccaacaacggttacgacgtggtgaacttgggcatcaagcagccactgtccgccatgttggaagcagcggaagaacacaaagcagacgtcatcggcatgtcgggacttcttgtgaagtccaccgtggtgatgaaggaaaaccttgaggagatgaacaacgccggcgcatccaattacccagtcattttgggtggcgctgcgctgacgcgtacctacgtggaaaacgatctcaacgaggtgtacaccggtgaggtgtactacgcccgtgatgctttcgagggcctgcgcctgatggatgaggtgatggcagaaaagcgtggtgaaggacttgatcccaactcaccagaagctattgagcaggcgaagaagaaggcggaacgtaaggctcgtaatgagcgttcccgcaagattgccgcggagcgtaaagctaatgcggctcccgtgattgttccggagcgttctgatgtctccaccgatactccaaccgcggcaccaccgttctggggaacccgcattgtcaagggtctgcccttggcggagttcttgggcaaccttgatgagcgcgccttgttcatggggcagtggggtctgaaatccacccgcggcaacgagggtccaagctatgaggatttggtggaaactgaaggccgaccacgcctgcgctactggctggatcgcctgaagtctgagggcattttggaccacgtggccttggtgtatggctacttcccagcggtcgcggaaggcgatgacgtggtgatcttggaatccccggatccacacgcagccgaacgcatgcgctttagcttcccacgccagcagcgcggcaggttcttgtgcatcgcggatttcattcgcccacgcgagcaagctgtcaaggacggccaagtggacgtcatgccattccagctggtcaccatgggtaatcctattgctgatttcgccaacgagttgttcgcagccaatgaataccgcgagtacttggaagttcacggcatcggcgtgcagctcaccgaagcattggccgagtactggcactcccgagtgcgcagcgaactcaagctgaacgacggtggatctgtcgctgattttgatccagaagacaagaccaagttcttcgacctggattaccgcggcgcccgcttctcctttggttacggttcttgccctgatctggaagaccgcgcaaagctggtggaattgctcgagccaggccgtatcggcgtggagttgtccgaggaactccagctgcacccagagcagtccacagacgcgtttgtgctctaccacccagaggcaaagtactttaacgtctaatctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 4 - DNA, 184 bp, Artificial Sequence
  Description of Artificial Sequence Synthetic promoter sequence
ggtcgagcggcttaaagtttggctgccatgtgaatttttagcaccctcaacagttgagtgctggcactctcgggggtagagtgccaaataggttgtttgacacacagttgttcacccgcgacgacggctgtgctggaaacccacaaccggcacacacaaaatttttctcatggagggattcatc

SEQ ID NO: 5 - DNA, 7070 bp, Artificial Sequence
  Description of Artificial Sequence Synthetic nucleotide sequence
tcgagaggcctgacgtcgggcccggtaccacgcgtcatatgactagttggagaatcatgacctcagcatctgccccaagctttaaccccggcaagggtcccggctcagcagtcggaattgcccttttaggattcggaacagtcggcactgaggtgatgcgtctgatgaccgagtacggtgatgaacttgcgcaccgcattggtggcccactggaggttcgtggcattgctgtttctgatatctcaaagccacgtgaaggcgttgcacctgagctgctcactgaggacgcttttgcactcatcgagcgcgaggatgttgacatcgtcgttgaggttatcggcggcattgagtacccacgtgaggtagttctcgcagctctgaaggccggcaagtctgttgttaccgccaataaggctcttgttgcagctcactctgctgagcttgctgatgcagcggaagccgcaaacgttgacctgtacttcgaggctgctgttgcaggcgcaattccagtggttggcccactgcgtcgctccctggctggcgatcagatccagtctgtgatgggcatcgttaacggcaccaccaacttcatcttggacgccatggattccaccggcgctgactatgcagattctttggctgaggcaactcgtttgggttacgccgaagctgatccaactgcagacgtcgaaggccatgacgccgcatccaaggctgcaattttggcatccatcgctttccacacccgtgttaccgcggatgatgtgtactgcgaaggtatcagcaacatcagcgctgccgacattgaggcagcacagcaggcaggccacaccatcaagttgttggccatctgtgagaagttcaccaacaaggaaggaaagtcggctatttctgctcgcgtgcacccgactctattacctgtgtcccacccactggcgtcggtaaacaagtcctttaatgcaatctttgttgaagcagaagcagctggtcgcctgatgttctacggaaacggtgcaggtggcgcgccaaccgcgtctgctgtgcttggcgacgtcgttggtgccgcacgaaacaaggtgcacggtggccgtgctccaggtgagtccacctacgctaacctgccgatcgctgatttcggtgagaccaccactcgttaccacctcgacatggatgtggaagatcgcgtgggggttttggctgaattggctagcctgttctctgagcaaggaatcttcctgcgtacaatccgacaggaagagcgcgatgatgatgcacgtctgatcgtggtcacccactctgcgctggaatctgatctttcccgcaccgttgaactgctgaaggctaagcctgttgttaaggcaatcaacagtgtgatccgcctcgaaagggactaattttactgacatggcaattgaactgaacgtcggtcgtaaggttaccgtcacggtacctggatcttctgcaaacctcggacctggctttgacactttaggtttggcactgtcggtatacgacactgtcgaagtggaaattattccatctggcttggaagtggaagtttttggcgaaggccaaggcgaagtccctcttgatggctcccacctggtggttaaagctattcgtgctggcctgaaggcagctgacgctgaagttcctggattgcgagtggtgtgccacaacaacattccgcagtctcgtggtcttggctccgctgctgcagcggcggttgctggtgttgctgcagctaatggtttggcggatttcccgctgactcaagagcagattgttcagttgtcctctgcctttgaaggccacccagataatgctgcggcttctgtgctgggtggagcagtggtgtcgtggacaaatctgtctatcgacggcaagagccagccacagtatgctgctgtaccacttgaggtgcaggacaatattcgtgcgactgcgctggttcctaatttccacgcatccaccgaagctgtgcgccgagtccttcccactgaagtcactcacatcgatgcgcgatttaacgtgtcccgcgttgcagtgatgatcgttgcgttgcagcagcgtcctgatttgctgtgggagggtactcgtgaccgtctgcaccagccttatcgtgcagaagtgttgcctattacctctgagtgggtaaaccgcctgcgcaaccgtggctacgcggcatacctttccggtgccggcccaaccgccatggtgctgtccactgagccaattccagacaaggttttggaagatgctcgtgagtctggcattaaggtgcttgagcttgaggttgcgggaccagtcaaggttgaagttaaccaaccttaggcccaacaaggaaggcccccttcgaatcaagaagggggccttattagtgcagcaattattcgctgaacacgtgaaccttacaggtgcccggcgcgttgagtggtttgagttccagctggatgcggttgttttcaccgaggctttcttggatgaatccggcgtggatggcgcagacgaaggctgatgggcgtttgtcgttgaccacaaatgggcagctgtgtagagcgagggagtttgcttcttcggtttcggtggggtcaaagcccatttcgcggaggcggttaatgagcggggagagggcttcgtcgagttcttcggcttcggcgtggttaatgcccatgacgtgtgcccactgggttccgatggaaagtgctttggcgcggaggtcggggttgtgcattgcgtcatcgtcgacatcgccgagcatgttggccatgagttcgatcagggtgatgtattctttggcgacagcgcggttgtcggggacgcgtgtttggaagatgagggaggggcgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 6 - DNA, 6625 bp, Artificial Sequence
  Description of Artificial Sequence Synthetic nucleotide sequence
tcgagaggcctgacgtcgggcccggtaccgttgctcgctgatctttcggcttaacaactttgtattcaatcagtcgggcatagaaagaaaacgcaatgatataggaaccaactgccgccaaaaccagccacacagagttgattgtttcgccacgggagaaagcgattgctccccaacccaccgccgcgataaccccaaagacaaggagaccaacgcgggcggtcggtgacattttaggggacttcttcacgcctactggaaggtcagtagcgttgctgtacaccaaatcatcgtcattgatgttgtcagtctgttttatggtcacgatctttactgttttctcttcgggtcgtttcaaagccactatgcgtagaaacagcgggcagaaacagcgggcagaaactgtgtgcagaaatgcatgcagaaaaaggaaagttcggccagatgggtgtttctgtatgccgatgatcggatctttgacagctgggtatgcgacaaatcaccgagagttgttaattcttaacaatggaaaagtaacattgagagatgatttataccatcctgcaccatttagagtggggctagtcatacccccataaccctagctgtacgcaatcgatttcaaatcagttggaaaaagtcaagaaaattacccgagacatatgcggcttaaagtttggctgccatgtgaatttttagcaccctcaacagttgagtgctggcactctcgagggtagagtgccaaataggttgtttgacacacagttgttcacccgcgacgacggctgtgctggaaacccacaaccggcacacacaaaatttttctcatggccgttaccctgcgaatgtccacagggtagctggtagtttgaaaatcaacgccgttgcccttaggattcagtaactggcacattttgtaatgcgctagatctgtgtgctcagtcttccaggctgcttatcacagtgaaagcaaaaccaattcgtggctgcgaaagtcgtagccaccacgaagtccaggaggacatacaatgccaaagtacgacaattccaatgctgaccagtggggctttgaaacccgctccattcacgcaggccagtcagtagacgcacagaccagcgcacgaaaccttccgatctaccaatccaccgctttcgtgttcgactccgctgagcacgccaagcagcgtttcgcacttgaggatctaggccctgtttactcccgcctcaccaacccaaccgttgaggctttggaaaaccgcatcgcttccctcgaaggtggcgtccacgctgtagcgttctcctccggacaggccgcaaccaccaacgccattttgaacctggcaggagcgggcgaccacatcgtcacctccccacgcctctacggtggcaccgagactctattccttatcactcttaaccgcctgggtatcgatgtttccttcgtggaaaaccccgacgaccctgagtcctggcaggcagccgttcagccaaacaccaaagcattcttcggcgagactttcgccaacccacaggcagacgtcctggatattcctgcggtggctgaagttgcgcaccgcaacagcgttccactgatcatcgacaacaccatcgctaccgcagcgctcgtgcgcccgctcgagctcggcgcagacgttgtcgtcgcttccctcaccaagttctacaccggcaacggctccggactgggcggcgtgcttatcgacggcggaaagttcgattggactgtcgaaaaggatggaaagccagtattcccctacttcgtcactccagatgctgcttaccacggattgaagtacgcagaccttggtgcaccagccttcggcctcaaggttcgcgttggccttctacgcgacaccggctccaccctctccgcattcaacgcatgggctgcagtccagggcatcgacaccctttccctgcgcctggagcgccacaacgaaaacgccatcaaggttgcagaattcctcaacaaccacgagaaggtggaaaaggttaacttcgcaggcctgaaggattccccttggtacgcaaccaaggaaaagcttggcctgaagtacaccggctccgttctcaccttcgagatcaagggcggcaaggatgaggcttgggcatttatcgacgccctgaagctacactccaaccttgcaaacatcggcgatgttcgctccctcgttgttcacccagcaaccaccacccattcacagtccgacgaagctggcctggcacgcgcgggcgttacccagtccaccgtccgcctgtccgttggcatcgagaccattgatgatatcatcgctgacctcgaaggcggctttgctgcaatctagcactagttcggacctagggatatcgtcgacatcgatgctcttctgcgttaattaacaattgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 7 - DNA, 363 bp, Artificial Sequence
  Description of Artificial Sequence Synthetic promoter sequence
cggcttaaagtttggctgccatgtgaatttttagcaccctcaacagttgagtgctggcactctcgagggtagagtgccaaataggttgtttgacacacagttgttcacccgcgacgacggctgtgctggaaacccacaaccggcacacacaaaatttttctcatggccgttaccctgcgaatgtccacagggtagctggtagtttgaaaatcaacgccgttgcccttaggattcagtaactggcacattttgtaatgcgctagatctgtgtgctcagtcttccaggctgcttatcacagtgaaagcaaaaccaattcgtggctgcgaaagtcgtagccaccacgaagtccaggaggacataca

SEQ ID NO: 8 - DNA, 6350 bp, Artificial Sequence
  Description of Artificial Sequence Synthetic nucleotide sequence
tcgagctcggcgcagacgttgtcgtcgcttccctcaccaagttctacaccggcaacggctccggactgggcggcgtgcttatcgacggcggaaagttcgattggactgtcgaaaaggatggaaagccagtattcccctacttcgtcactccagatgctgcttaccacggattgaagtacgcagaccttggtgcaccagccttcggcctcaaggttcgcgttggccttctacgcgacaccggctccaccctctccgcattcaacgcatgggctgcagtccagggcatcgacaccctttccctgcgcctggagcgccacaacgaaaacgccatcaaggttgcagaattcctcaacaaccacgagaaggtggaaaaggttaacttcgcaggcctgaaggattccccttggtacgcaaccaaggaaaagcttggcctgaagtacaccggctccgttctcaccttcgagatcaagggcggcaaggatgaggcttgggcatttatcgacgccctgaagctacactccaaccttgcaaacatcggcgatgttcgctccctcgttgttcacccagcaaccaccacccattcacagtccgacgaagctggcctggcacgcgcgggcgttacccagtccaccgtccgcctgtccgttggcatcgagaccattgatgatatcatcgctgacctcgaaggcggctttgctgcaatctagcactagttcggacctagggatatcgtcgagagctgccaattattccgggcttgtgacccgctacccgataaataggtcggctgaaaaatttcgttgcaatatcaacaaaaaggcctatcattgggaggtgtcgcaccaagtacttttgcgaagcgccatctgacggattttcaaaagatgtatatgctcggtgcggaaacctacgaaaggattttttacccatgcccaccctcgcgccttcaggtcaacttgaaatccaagcgatcggtgatgtctccaccgaagccggagcaatcattacaaacgctgaaatcgcctatcaccgctggggtgaataccgcgtagataaagaaggacgcagcaatgtcgttctcatcgaacacgccctcactggagattccaacgcagccgattggtgggctgacttgctcggtcccggcaaagccatcaacactgatatttactgcgtgatctgtaccaacgtcatcggtggttgcaacggttccaccggacctggctccatgcatccagatggaaatttctggggtaatcgcttccccgccacgtccattcgtgatcaggtaaacgccgaaaaacaattcctcgacgcactcggcatcaccacggtcgccgcagtacttggtggttccatgggtggtgcccgcaccctagagtgggccgcaatgtacccagaaactgttggcgcagctgctgttcttgcagtttctgcacgcgccagcgcctggcaaatcggcattcaatccgcccaaattaaggcgattgaaaacgaccaccactggcacgaaggcaactactacgaatccggctgcaacccagccaccggactcggcgccgcccgacgcatcgcccacctcacctaccgtggcgaactagaaatcgacgaacgcttcggcaccaaagcccaaaagaacgaaaacccactcggtccctaccgcaagcccgaccagcgcttcgccgtggaatcctacttggactaccaagcagacaagctagtacagcgtttcgacgccggctcctacgtcttgctcaccgacgccctcaaccgccacgacattggtcgcgaccgcggaggcctcaacaaggcactcgaatccatcaaagttccagtccttgtcgcaggcgtagataccgatattttgtacccctaccaccagcaagaacacctctccagaaacctgggaaatctactggcaatggcaaaaatcgtatcccctgtcggccacgatgctttcctcaccgaaagccgccaaatggatcgcatcgtgaggaacttcttcagcctcatctccccagacgaagacaacccttcgacctacatcgagttctacatctaacatatgactagttcggacctagggatatcgtcgacatcgatgctcttctgcgttaattaacaattgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 9 - DNA, 192 bp, Artificial Sequence
  Description of Artificial Sequence Synthetic promoter sequence
gagctgccaattattccgggcttgtgacccgctacccgataaataggtcggctgaaaaatttcgttgcaatatcaacaaaaaggcctatcattgggaggtgtcgcaccaagtacttttgcgaagcgccatctgacggattttcaaaagatgtatatgctcggtgcggaaacctacgaaaggattttttaccc

SEQ ID NO: 10 - DNA, 7080 bp, Artificial Sequence
  Description of Artificial Sequence Synthetic nucleotide sequence
cagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgctcccggccgggccatgggccgggcgtgcgcaatagactcgtcaccaaaaccgatggagtgtttttgacgctggaagatggcagcaccgtgattgacgcgatgagctcctggtggtcggcaattcatggacacggacacccccgactgaaagctgccgcccaaaaacaaatcgacaccatgagtcacgtcatgtttggcggactaacccacgagcccgccattaagctcacccacaaactcctcaatctcactggaaattcctttgaccacgtcttttattccgattcgggctcggtctcagtggaggtcgccatcaaaatggcactgcaggcctccaaaggacaaggccacccggaacgaacaaaactcctcacctggcggtccggctaccacggagacacattcaccgcgatgagcgtgtgcgacccagaaaatggcatgcatagcctctggaaaggcacactccccgagcagattttcgcccccgccccaccagttcgggggtcatcgccgcaggcgatttccgagtacctgcgcagcatggaattgcttatcgacgagaccgtctccgcaatcatcatcgaaccgatcgtccaaggcgctggaggcatgcgcgcggccgcacagcgatcccagaggaaatatcctctggggtcgctgtgtcgaccttaaagtttggctgccatgtgaatttttagcaccctcaacagttgagtgctggcactctcgggggtagagtgccaaataggttgtttgacacacagttgttcacccgcgacgacggctgtgctggaaacccacaaccggcacacacaaaatttttctagtaggagcacaaacacatgtccctaacgaacatcccagcctcatctcaatgggcaattagcgacgttttgaagcgtccttcacccggccgagtacctttttctgtcgagtttatgccaccccgcgacgatgcagctgaagagcgtctttaccgcgcagcagaggtcttccatgacctcggtgcatcgtttgtctccgtgacttatggtgctggcggatcaacccgtgagagaacctcacgtattgctcgacgattagcgaaacaaccgttgaccactctggtgcacctgaccctggttaaccacactcgcgaagagatgaaggcaattcttcgggaatacctagagctgggattaacaaacctgttggcgcttcgaggagatccgcctggagacccattaggcgattgggtgagcaccgatggaggactgaactatgcctctgagctcatcgatcttattaagtccactcctgagttccgggaattcgacctcggtatcgcctccttccccgaagggcatttccgggcgaaaactctagaagaagacaccaaatacactctggcgaagctgcgtggaggggcagagtactccatcacgcagatgttctttgatgtggaagactacctgcgacttcgtgatcgccttgtcgctgcagaccccattcatggtgcgaagccaatcattcctggcatcatgcccattacgagcctgcggtctgtgcgtcgacaggtcgaactctctggtgctcaattgccgagccaactagaagaatcacttgttcgagctgcaaacggcaatgaagaagcgaacaaagacgagatccgcaaggtgggcattgaatattccaccaatatggcagagcgactcattgccgaaggtgcggaagatctgcacttcatgacgcttaacttcacccgtgcaacccaagaagtgttgtacaaccttggcatggcgcctgcttggggagcagagcacggccaagacgcggtgcgttaagggatccgccctcccgcacgctttgcgggagggcggtaccggaactggggttgggaaaaccttctccacagccgttttggttcgatacttagccgatcaaggacacgatgttctgcccgtaaagctagtccaaaccggtgaacttccaggcgagggagacatctttaacattgaacgcttgactggaattgctggagaggaatttgctcgtttcaaagaccctcttgcgccaaatctggcagcccgacgagagggggtcgagccaatacagtttgatcagattatctcgtggcttcgtggttttgacgacccagatcgcatcattgtggtggagggcgctggtggcctgctggtcagattaggggaagatttcaccctggcagatgttgcctccgctttgaatgcacccttagtgattgtgacaagcaccggattgggaagcctcaacgctgctgaattaagcgttgaggcagcaaaccgccgaggactcacagtgttgggagtcctcggcggttcgatccctcaaaatcctgatctagctacgatgcttaatctcgaagaatttgagagagtcaccggcgtgcccttttggggagctttgccggaagggttgtcacgggtggaggggttcgtcgaaaagcaatcttttccggcccttgatgcctttaagaaaccgccggcaaggctcccaacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgattt

SEQ ID NO: 11 - DNA, 8554 bp, Artificial Sequence
  Description of Artificial Sequence Synthetic nucleotide sequence
tcgagaggcctgacgtcgggcccggtaccgttgctcgctgatctttcggcttaacaactttgtattcaatcagtcgggcatagaaagaaaacgcaatgatataggaaccaactgccgccaaaaccagccacacagagttgattgtttcgccacgggagaaagcgattgctccccaacccaccgccgcgataaccccaaagacaaggagaccaacgcgggcggtcggtgacattttaggggacttcttcacgcctactggaaggtcagtagcgttgctgtacaccaaatcatcgtcattgatgttgtcagtctgttttatggtcacgatctttactgttttctcttcgggtcgtttcaaagccactatgcgtagaaacagcgggcagaaactgtgtgcagaaatgcatgcagaaaaaggaaagttcggccagatgggtgtttctgtatgccgatgatcggatctttgacagctgggtatgcgacaaatcaccgagagttgttaattcttaacaatggaaaagtaacattgagagatgatttataccatcctgcaccatttagagtggggctagtcatacccccataaccctagctgtacgcaatcgatttcaaatcagttggaaaaagtcaagaaaattacccgagacatatgcggcttaaagtttggctgccatgtgaatttttagcaccctcaacagttgagtgctggcactctcgagggtagagtgccaaataggttgtttgacacacagttgttcacccgcgacgacggctgtgctggaaacccacaaccggcacacacaaaatttttctcatggagggattcatcatgccaaagtacgacaattccaatgctgaccagtggggctttgaaacccgctccattcacgcaggccagtcagtagacgcacagaccagcgcacgaaaccttccgatctaccaatccaccgctttcgtgttcgactccgctgagcacgccaagcagcgtttcgcacttgaggatctaggccctgtttactcccgcctcaccaacccaaccgttgaggctttggaaaaccgcatcgcttccctcgaaggtggcgtccacgctgtagcgttctcctccggacaggccgcaaccaccaacgccattttgaacctggcaggagcgggcgaccacatcgtcacctccccacgcctctacggtggcaccgagactctattccttatcactcttaaccgcctgggtatcgatgtttccttcgtggaaaaccccgacgaccctgagtcctggcaggcagccgttcagccaaacaccaaagcattcttcggcgagactttcgccaacccacaggcagacgtcctggatattcctgcggtggctgaagttgcgcaccgcaacagcgttccactgatcatcgacaacaccatcgctaccgcagcgctcgtgcgcccgctcgagctcggcgcagacgttgtcgtcgcttccctcaccaagttctacaccggcaacggctccggactgggcggcgtgcttatcgacggcggaaagttcgattggactgtcgaaaaggatggaaagccagtattcccctacttcgtcactccagatgctgcttaccacggattgaagtacgcagaccttggtgcaccagccttcggcctcaaggttcgcgttggccttctacgcgacaccggctccaccctctccgcattcaacgcatgggctgcagtccagggcatcgacaccctttccctgcgcctggagcgccacaacgaaaacgccatcaaggttgcagaattcctcaacaaccacgagaaggtggaaaaggttaacttcgcaggcctgaaggattccccttggtacgcaaccaaggaaaagcttggcctgaagtacaccggctccgttctcaccttcgagatcaagggcggcaaggatgaggcttgggcatttatcgacgccctgaagctacactccaaccttgcaaacatcggcgatgttcgctccctcgttgttcacccagcaaccaccacccattcacagtccgacgaagctggcctggcacgcgcgggcgttacccagtccaccgtccgcctgtccgttggcatcgagaccattgatgatatcatcgctgacctcgaaggcggctttgctgcaatctagcactagttcggacctagggatatcgtcgagagctgccaattattccgggcttgtgacccgctacccgataaataggtcggctgaaaaatttcgttgcaatatcaacaaaaaggcctatcattgggaggtgtcgcaccaagtacttttgcgaagcgccatctgacggattttcaaaagatgtatatgctcggtgcggaaacctacgaaaggattttttacccatgcccaccctcgcgccttcaggtcaacttgaaatccaagcgatcggtgatgtctccaccgaagccggagcaatcattacaaacgctgaaatcgcctatcaccgctggggtgaataccgcgtagataaagaaggacgcagcaatgtcgttctcatcgaacacgccctcactggagattccaacgcagccgattggtgggctgacttgctcggtcccggcaaagccatcaacactgatatttactgcgtgatctgtaccaacgtcatcggtggttgcaacggttccaccggacctggctccatgcatccagatggaaatttctggggtaatcgcttccccgccacgtccattcgtgatcaggtaaacgccgaaaaacaattcctcgacgcactcggcatcaccacggtcgccgcagtacttggtggttccatgggtggtgcccgcaccctagagtgggccgcaatgtacccagaaactgttggcgcagctgctgttcttgcagtttctgcacgcgccagcgcctggcaaatcggcattcaatccgcccaaattaaggcgattgaaaacgaccaccactggcacgaaggcaactactacgaatccggctgcaacccagccaccggactcggcgccgcccgacgcatcgcccacctcacctaccgtggcgaactagaaatcgacgaacgcttcggcaccaaagcccaaaagaacgaaaacccactcggtccctaccgcaagcccgaccagcgcttcgccgtggaatcctacttggactaccaagcagacaagctagtacagcgtttcgacgccggctcctacgtcttgctcaccgacgccctcaaccgccacgacattggtcgcgaccgcggaggcctcaacaaggcactcgaatccatcaaagttccagtccttgtcgcaggcgtagataccgatattttgtacccctaccaccagcaagaacacctctccagaaacctgggaaatctactggcaatggcaaaaatcgtatcccctgtcggccacgatgctttcctcaccgaaagccgccaaatggatcgcatcgtgaggaacttcttcagcctcatctccccagacgaagacaacccttcgacctacatcgagttctacatctaacatatgactagttcggacctagggatatcgtcgacatcgatgctcttctgcgttaattaacaattgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgcgcaaagtcccgcttcgtgaaaattttcgtgccgcgtgattttccgccaaaaactttaacgaacgttcgttataatggtgtcatgaccttcacgacgaagtactaaaattggcccgaatcatcagctatggatctctctgatgtcgcgctggagtccgacgcgctcgatgctgccgtcgatttaaaaacggtgatcggatttttccgagctctcgatacgacggacgcgccagcatcacgagactgggccagtgccgcgagcgacctagaaactctcgtggcggatcttgaggagctggctgacgagctgcgtgctcggccagcgccaggaggacgcacagtagtggaggatgcaatcagttgcgcctactgcggtggcctgattcctccccggcctgacccgcgaggacggcgcgcaaaatattgctcagatgcgtgtcgtgccgcagccagccgcgagcgcgccaacaaacgccacgccgaggagctggaggcggctaggtcgcaaatggcgctggaagtgcgtcccccgagcgaaattttggccatggtcgtcacagagctggaagcggcagcgagaattatcgcgatcgtggcggtgcccgcaggcatgacaaacatcgtaaatgccgcgtttcgtgtgccgtggccgcccaggacgtgtcagcgccgccaccacctgcaccgaatcggcagcagcgtcgcgcgtcgaaaaagcgcacaggcggcaagaagcgataagctgcacgaatacctgaaaaatgttgaacgccccgtgagcggtaactcacagggcgtcggctaacccccagtccaaacctgggagaaagcgctcaaaaatgactctagcggattcacgagacattgacacaccggcctggaaattttccgctgatctgttcgacacccatcccgagctcgcgctgcgatcacgtggctggacgagcgaagaccgccgcgaattcctcgctcacctgggcagagaaaatttccagggcagcaagacccgcgacttcgccagcgcttggatcaaagacccggacacggagaaacacagccgaagttataccgagttggttcaaaatcgcttgcccggtgccagtatgttgctctgacgcacgcgcagcacgcagccgtgcttgtcctggacattgatgtgccgagccaccaggccggcgggaaaatcgagcacgtaaaccccgaggtctacgcgattttggagcgctgggcacgcctggaaaaagcgccagcttggatcggcgtgaatccactgagcgggaaatgccagctcatctggctcattgatccggtgtatgccgcagcaggcatgagcagcccgaatatgcgcctgctggctgcaacgaccgaggaaatgacccgcgttttcggcgctgaccaggctttttcacataggctgagccgtggccactgcactctccgacgatcccagccgtaccgctggcatgcccagcacaatcgcgtggatcgcctagctgatcttatggaggttgctcgcatgatctcaggcacagaaaaacctaaaaaacgctatgagcaggagttttctagcggacgggcacgtatcgaagcggcaagaaaagccactgcggaagcaaaagcacttgccacgcttgaagcaagcctgccgagcgccgctgaagcgtctggagagctgatcgacggcgtccgtgtcctctggactgctccagggcgtgccgcccgtgatgagacggcttttcgccacgctttgactgtgggataccagttaaaagcggctggtgagcgcctaaaagacaccaagggtcatcgagcctacgagcgtgcctacaccgtcgctcaggcggtcggaggaggccgtgagcctgatctgccgccggactgtgaccgccagacggattggccgcgacgtgtgcgcggctacgtcgctaaaggccagccagtcgtccctgctcgtcagacagagacgcagagccagccgaggcgaaaagctctggccactatgggaagacgtggcggtaaaaaggccgcagaacgctggaaagacccaaacagtgagtacgcccgagcacagcgagaaaaactagctaagtccagtcaacgacaagctaggaaagctaaaggaaatcgcttgaccattgcaggttggtttatgactgttgagggagagactggctcgtggccgacaatcaatgaagctatgtctgaatttagcgtgtcacgtcagaccgtgaatagagcacttaaggtctgcgggcattgaacttccacgaggacgccgaaagcttcccagtaaatgtgccatctcgtaggcagaaaacggttcccccgtagggtctctctcttggcctcctttctaggtcgggctgattgctcttgaagctctctaggggggctcacaccataggcagataacgttccccaccggctcgcctcgtaagcgcacaaggactgctcccaaagatcttcaaagccactgccgcgactgccttcgcgaagccttgccccgcggaaatttcctccaccgagttcgtgcacacccctatgccaagcttctttcaccctaaattcgagagattggattcttaccgtggaaattcttcgcaaaaatcgtcccctgatcgcccttgcgacgttggcgtcggtgccgctggttgcgcttggcttgaccgacttgatcagcggccgctcgatttaaatc

SEQ ID NO: 12 - DNA, 8431 bp, Artificial Sequence
  Description of Artificial Sequence Synthetic nucleotide sequence
tcgaccaaaggacgcaccgatgagctgagctgctgcagtggattggcccaccatacccaaaggaatccatgatgttggtgttgccatcagtgggatgccctgcgccttggggccgaaatagtagaaatacactcgggtaaaaactgctggtgcagacgccaaagttaaaaggaagagcccgaaagaaacccacagcatcgccggaagttcaaagtgctcatggagttgtgctgccgaggtggaagcaaccatcggcgtgacaagaggaagaccccacgcaaaagttggtgtgcccgccttagatcgcaaaatggccgttatatataaggaataggcaacaagtcccacggctgtgccaatagaccagcacacaaacataaatccccacagatcatcacccaaaactacggggcttgcagttcccaatgcgatcaaacccatggacagcattgcccatgccggcatgacttcagttttgaatgaaggagagcggtagattagccaaccgccaataatgacaattgccaccacaacagctaacgcgaagaagaaatctgcgacgactggaaaaccatggattttcaacagtgatgacaacaatgagatgcccatgagggaaccagcccacgaggggccaggtggaggtaagaccgcagcgtagcttttggtcgaagaaggagtgggcatgcccattactttaagcctttggggcagtgaaaccgctaaatgggagcgttgtgcgctcgatcactggtctagacctttgggctccaaaagttgcaatttcgcgaatacttcaacacttgtttgcaatgtttgttaataaatgggttcgctagtggattctgtcgttagtactggccgtcgtggtggggtcatgtatttaggtagggcaaagttaagatcagagcactttttgatacgactaactggatataacctttggggtaacgtggggatgtgtgtgagtaattttcaaagtatttaaaagggggatctagggtaaaaatttggcttcaagtacatatctttagttcggtagttgagggcgggtggtgacagtgcgggcatgcatgtgagtgtaaatgttgttttaaaaaggtgtgtactgacagtgggccggtttgtgctggtcggccactagcggagtgcttggattgtgatggcagggtaagggaaagggattaccattaccgctgttcttggcgttttgttgcctattgtccgaatgttaagtgttaatggtgggaaaactgggaaagttgtcccctggaatgtgtgagaattgcccaaatctgaacccaatggccatggacggggaatgaactgtcggagaacggttgaggttaattcttgaaaccacccccaaaataggctatttaaacgggtgctctcatattaaagaaagtgtgtagatgcgtgtgggcagggggtaggtccactggtaatgacaaatgtgtccgttgtctcacctaaagctcgagcggcttaaagtttggctgccatgtgaatttttagcaccctcaacagttgagtgctggcactctcgggggtagagtgccaaataggttgtttgacacacagttgttcacccgcgacgacggctgtgctggaaacccacaaccggcacacacaaaatttttctcatgaagggattgcatatgacctcagcatctgccccaagctttaaccccggcaagggtcccggctcagcagtcggaattgcccttttaggattcggaacagtcggcactgaggtgatgcgtctgatgaccgagtacggtgatgaacttgcgcaccgcattggtggcccactggaggttcgtggcattgctgtttctgatatctcaaagccacgtgaaggcgttgcacctgagctgctcactgaggacgcttttgcactcatcgagcgcgaggatgttgacatcgtcgttgaggttatcggcggcattgagtacccacgtgaggtagttctcgcagctctgaaggccggcaagtctgttgttaccgccaataaggctcttgttgcagctcactctgctgagcttgctgatgcagcggaagccgcaaacgttgacctgtacttcgaggctgctgttgcaggcgcaattccagtggttggcccactgcgtcgctccctggctggcgatcagatccagtctgtgatgggcatcgttaacggcaccaccaacttcatcttggacgccatggattccaccggcgctgactatgcagattctttggctgaggcaactcgtttgggttacgccgaagctgatccaactgcagacgtcgaaggccatgacgccgcatccaaggctgcaattttggcatccatcgctttccacacccgtgttaccgcggatgatgtgtactgcgaaggtatcagcaacatcagcgctgccgacattgaggcagcacagcaggcaggccacaccatcaagttgttggccatctgtgagaagttcaccaacaaggaaggaaagtcggctatttctgctcgcgtgcacccgactctattacctgtgtcccacccactggcgtcggtaaacaagtcctttaatgcaatctttgttgaagcagaagcagctggtcgcctgatgttctacggaaacggtgcaggtggcgcgccaaccgcgtctgctgtgcttggcgacgtcgttggtgccgcacgaaacaaggtgcacggtggccgtgctccaggtgagtccacctacgctaacctgccgatcgctgatttcggtgagaccaccactcgttaccacctcgacatggatgtggaagatcgcgtgggggttttggctgaattggctagcctgttctctgagcaaggaatcttcctgcgtacaatccgacaggaagagcgcgatgatgatgcacgtctgatcgtggtcacccactctgcgctggaatctgatctttcccgcaccgttgaactgctgaaggctaagcctgttgttaaggcaatcaacagtgtgatccgcctcgaaagggactaaactagtagttcacgcttaagaactgctaaataacaagaaaggctcccaccgaaagtgggagcctttcttgtcgttaagcgatgaattcctcaaaacctcagtgctttttaaacaccaacaccaagttacttaccgcgaattctcggagcactgggactttaaccatccaccagacccaatacgggtggtagcggggaaaagcggcaaccaattccgcattgcccacggaggctccccattccagcccctcccggcaggacacattaaacagtgactccccgaaaacgttcttaggcgggtgcccgtgtttcttcgtgtagcgatcgcgggcaaattctccgccaacgtagtgttcccacagtccggtttcatggccgccgaagggccctaaccaaatggtgtagctcaggattgccaggccgccgctgcgggtgacgcggagcatttcttctcccaattcccacggtgcggagacatgttctgcaacgttggaggagtacaccacgtcaaaggaatcgggaagaaacggcaggtcgaggccggatccgcggactgatccgtggacgtcgatgccagctgcggacatttcgccaacgtcgggttcgacggagaagtaggtggcgcccagtgtctcaaaggcttcggcgaagtatccgggtccgccgccgacgtcgagaactttcaggtcatttaatccggcgccagaaatatcttcagacaaagccgccaccagactcgaggtatcgagggccaggtttccgtaaaagatgtcaggtcgggtttgttcgtatttgaaatcagacagtaaaccccacgacctgcccaaggtagccaagcgacgaagagccggaagctccggaaatgaggccatttatgcgcgggtccagttgaggtcgcggatgtcttcgccgttcatccatcgcaaaatggtggtgatggcatcgtcgatggaggaaacaatggtggtgttttttactcctgcgacgccgaattctagggtccagccgtctacttctgcgaggctgcgcgcgaagatgtaggaggactcgtcggcggggttgtctaggtcgaagtgtgcaatcttcaaataatccccggaatccccggaatctccagagcccccggcaatgacgttcaactggtgcagatcctctagagttctgtgaaaaacaccgtggggcagtttctgcttcgcggtgttttttatttgtggggcactagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttgagag

SEQ ID NO: 13 - DNA, 5863 bp, Artificial Sequence
  Description of Artificial Sequence Synthetic nucleotide sequence
tcgagaggcctgacgtcgggcccggtaccacgcgtcatatgcaggtgaggtaaccccaaaagaggtaaaacccgcgccaccgacttttcaggagcggggacgcgggtttttgccatgaatccgaagatactacatcagatttttaggccaacttgagggctgcgcgaagttcatcaacgcggtcgatagcctcccaaggaaggtccaaatcagtgcgaccaaagtggccgtaggcagcagtgtcagcgtagatcggacgaagcagatcaagctcacggataattgctgctggacgcaggtcaaagacctccaacacggcagcctgaatctgctcgtcgctcaggccttccttgttggtgtcaaaggtttcaacgtaaagtccgactggctttgcgcgtccaatggcgtatgcaacctgaacttcagcgcgatcagcaaggcctgctgccacgatgttctttgctacccaacgcatggcgtatgcagcagagcggtccaccttgcttggatccttaccggagaatgctccaccaccatggcgagccatgccaccgtaggtatccacgatgatcttgcggccggtcagacccgcatcacccatggggccacccagaatgaaggaacctgaagggttgatcaacacggtgatctcaccggttgccagatcctcaatgcctgcgtctttgattacccaatcaatgacgtgttcgcgcagttgggtttccaaccatgcacggtcaacttctgggtcgtgctgggtggagatgacaacggtatccaggtggctagggcggtcttgcgcatcgtatgcgaaggtgacctgggtttttccgtctggacgcaggtgaggaacgatgccctctttacgaacctgggtcagacgacgtgacagtcggtgcgccaacgcgataggaagaggcatgtactcttcggtttcgttggtggcgtagccgaacatcaggccctggtcgccagcacctgcgcggtcgtcttcttcaacgtcgccgttggtgcgggcttcgtcggagttatccacgccgtcagcgatttcctgggactgctcaccgatggatactgagacgccagcggtgcgtccgtcgaatccaacctcagaggagttgaatccgatttcgatgagcttgttgcggactaattgagggatctctacgtaagcgctggtacggacctcgccaacaacatggacgattccggtggtgaccacagtttccactgcgacgcgcgactgcggatctttttcgagcagcgcgtccaaaatggtatcggaaatagcatcacatattttgtctggatgtccctcagttacagattcactggtgaacaaacggacggcggttggctgagccacaaatacccttctttcgaagaagttgagaataaatagtcttaaatacaaaaaaccaatatagaccaagctgtctaaaactgcaatgtcagtggtctagctggatttttctagacttcgcgatacgccagtgccgcgtcccaaatttgcgcagcaacctcgattttgctgccgtgctccacatcgactaccccaccgtgagcatccaaaatccagccctcattgtgcttttgcccaaacactttgcccatgcccacctcattacacatgaggaggtcgcagcccttcttcccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 14 - DNA, 5477 bp, Artificial Sequence
  Description of Artificial Sequence Synthetic nucleotide sequence
tcgagctctccaatctccactgaggtacttaatccttccggggaattcgggcgcttaaatcgagaaattaggccatcaccttttaataacaatacaatgaataattggaataggtcgacacctttggagcggagccggttaaaattggcagcattcaccgaaagaaaaggagaaccacatgcttgccctaggttggattacatggatcattattggtggtctagctggttggattgcctccaagattaaaggcactgatgctcagcaaggaattttgctgaacatagtcgtcggtattatcggtggtttgttaggcggctggctgcttggaatcttcggagtggatgttgccggtggcggcttgatcttcagcttcatcacatgtctgattggtgctgtcattttgctgacgatcgtgcagttcttcactcggaagaagtaatctgctttaaatccgtagggcctgttgatatttcgatatcaacaggccttttggtcattttggggtggaaaaagcgctagacttgcctgtggattaaaactatacgaaccggtttgtctatattggtgttagacagttcgtcgtatcttgaaacagaccaacccgaaaggacgtggccgaacgtggctgctagctaatccttgatggtggacttgctggatctcgattggtccacaacatcagtcctcttgagacggctcgcgatttggctcggcagttgttgtcggctccacctgcggactactcaatttagtttcttcattttccgaaggggtatcttcgttgggggaggcgtcgataagccccttctttttagctttaacctcagcgcgacgctgctttaagcgctgcatggcggcgcggttcatttcacgttgcgtttcgcgcctcttgttcgcgatttctttgcgggcctgttttgcttcgttgatttcggcagtacgggttttggtgagttccacgtttgttgcgtgaagcgttgaggcgttccatggggtgagaatcatcagggcgcggtttttgcgtcgtgtccacaggaagatgcgcttttctttttgttttgcgcggtagatgtcgcgctgctctaggtggtgcactttgaaatcgtcggtaagtgggtatttgcgttccaaaatgaccatcatgatgattgtttggaggagcgtccacaggttgttgctgacgcgtcatatgactagttcggacctagggatatcgtcgacatcgatgctcttctgcgttaattaacaattgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 15 - DNA, 8877 bp, Artificial Sequence
  Description of Artificial Sequence Synthetic nucleotide sequence
tcgatttaaatctcgagaggcctgacgtcgggcccggtaccgggccccccctcgaggtcgagcggcttaaagtttggctgccatgtgaatttttagcaccctcaacagttgagtgctggcactctcgggggtagagtgccaaataggttgtttgacacacagttgttcacccgcgacgacggctgtgctggaaacccacaaccggcacacacaaaatttttctcatggagggattcatcatgtcgacttcagttacttcaccagcccacaacaacgcacattcctccgaatttttggatgcgttggcaaaccatgtgttgatcggcgacggcgccatgggcacccagctccaaggctttgacctggacgtggaaaaggatttccttgatctggaggggtgtaatgagattctcaacgacacccgccctgatgtgttgaggcagattcaccgcgcctactttgaggcgggagctgacttggttgagaccaatacttttggttgcaacctgccgaacttggcggattatgacatcgctgatcgttgccgtgagcttgcctacaagggcactgcagtggctagggaagtggctgatgagatggggccgggccgaaacggcatgcggcgtttcgtggttggttccctgggacctggaacgaagcttccatcgctgggccatgcaccgtatgcagatttgcgtgggcactacaaggaagcagcgcttggcatcatcgacggtggtggcgatgcctttttgattgagactgctcaggacttgcttcaggtcaaggctgcggttcacggcgttcaagatgccatggctgaacttgatacattcttgcccattatttgccacgtcaccgtagagaccaccggcaccatgctcatgggttctgagatcggtgccgcgttgacagcgctgcagccactgggtatcgacatgattggtctgaactgcgccaccggcccagatgagatgagcgagcacctgcgttacctgtccaagcacgccgatattcctgtgtcggtgatgcctaacgcaggtcttcctgtcctgggtaaaaacggtgcagaatacccacttgaggctgaggatttggcgcaggcgctggctggattcgtctccgaatatggcctgtccatggtgggtggttgttgtggcaccacacctgagcacatccgtgcggtccgcgatgcggtggttggtgttccagagcaggaaacctccacactgaccaagatccctgcaggccctgttgagcaggcctcccgcgaggtggagaaagaggactccgtcgcgtcgctgtacacctcggtgccattgtcccaggaaaccggcatttccatgatcggtgagcgcaccaactccaacggttccaaggcattccgtgaggcaatgctgtctggcgattgggaaaagtgtgtggatattgccaagcagcaaacccgcgatggtgcacacatgctggatctttgtgtggattacgtgggacgagacggcaccgccgatatggcgaccttggcagcacttcttgctaccagctccactttgccaatcatgattgactccaccgagccagaggttattcgcacaggccttgagcacttgggtggacgaagcatcgttaactccgtcaactttgaagacggcgatggccctgagtcccgctaccagcgcatcatgaaactggtaaagcagcacggtgcggccgtggttgcgctgaccattgatgaggaaggccaggcacgtaccgctgagcacaaggtgcgcattgctaaacgactgattgacgatatcaccggcagctacggcctggatatcaaagacatcgttgtggactgcctgaccttcccgatctctactggccaggaagaaaccaggcgagatggcattgaaaccatcgaagccatccgcgagctgaagaagctctacccagaaatccacaccaccctgggtctgtccaatatttccttcggcctgaaccctgctgcacgccaggttcttaactctgtgttcctcaatgagtgcattgaggctggtctggactctgcgattgcgcacagctccaagattttgccgatgaaccgcattgatgatcgccagcgcgaagtggcgttggatatggtctatgatcgccgcaccgaggattacgatccgctgcaggaattcatgcagctgtttgagggcgtttctgctgccgatgccaaggatgctcgcgctgaacagctggccgctatgcctttgtttgagcgtttggcacagcgcatcatcgacggcgataagaatggccttgaggatgatctggaagcaggcatgaaggagaagtctcctattgcgatcatcaacgaggaccttctcaacggcatgaagaccgtgggtgagctgtttggttccggacagatgcagctgccattcgtgctgcaatcggcagaaaccatgaaaactgcggtggcctatttggaaccgttcatggaagaggaagcagaagctaccggatctgcgcaggcagagggcaagggcaaaatcgtcgtggccaccgtcaagggtgacgtgcacgatatcggcaagaacttggtggacatcattttgtccaacaacggttacgacgtggtgaacttgggcatcaagcagccactgtccgccatgttggaagcagcggaagaacacaaagcagacgtcatcggcatgtcgggacttcttgtgaagtccaccgtggtgatgaaggaaaaccttgaggagatgaacaacgccggcgcatccaattacccagtcattttgggtggcgctgcgctgacgcgtacctacgtggaaaacgatctcaacgaggtgtacaccggtgaggtgtactacgcccgtgatgctttcgagggcctgcgcctgatggatgaggtgatggcagaaaagcgtggtgaaggacttgatcccaactcaccagaagctattgagcaggcgaagaagaaggcggaacgtaaggctcgtaatgagcgttcccgcaagattgccgcggagcgtaaagctaatgcggctcccgtgattgttccggagcgttctgatgtctccaccgatactccaaccgcggcaccaccgttctggggaacccgcattgtcaagggtctgcccttggcggagttcttgggcaaccttgatgagcgcgccttgttcatggggcagtggggtctgaaatccacccgcggcaacgagggtccaagctatgaggatttggtggaaactgaaggccgaccacgcctgcgctactggctggatcgcctgaagtctgagggcattttggaccacgtggccttggtgtatggctacttcccagcggtcgcggaaggcgatgacgtggtgatcttggaatccccggatccacacgcagccgaacgcatgcgctttagcttcccacgccagcagcgcggcaggttcttgtgcatcgcggatttcattcgcccacgcgagcaagctgtcaaggacggccaagtggacgtcatgccattccagctggtcaccatgggtaatcctattgctgatttcgccaacgagttgttcgcagccaatgaataccgcgagtacttggaagttcacggcatcggcgtgcagctcaccgaagcattggccgagtactggcactcccgagtgcgcagcgaactcaagctgaacgacggtggatctgtcgctgattttgatccagaagacaagaccaagttcttcgacctggattaccgcggcgcccgcttctcctttggttacggttcttgccctgatctggaagaccgcgcaaagctggtggaattgctcgagccaggccgtatcggcgtggagttgtccgaggaactccagctgcacccagagcagtccacagacgcgtttgtgctctaccacccagaggcaaagtactttaacgtctaatctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgcgcaaagtcccgcttcgtgaaaattttcgtgccgcgtgattttccgccaaaaactttaacgaacgttcgttataatggtgtcatgaccttcacgacgaagtactaaaattggcccgaatcatcagctatggatctctctgatgtcgcgctggagtccgacgcgctcgatgctgccgtcgatttaaaaacggtgatcggatttttccgagctctcgatacgacggacgcgccagcatcacgagactgggccagtgccgcgagcgacctagaaactctcgtggcggatcttgaggagctggctgacgagctgcgtgctcggccagcgccaggaggacgcacagtagtggaggatgcaatcagttgcgcctactgcggtggcctgattcctccccggcctgacccgcgaggacggcgcgcaaaatattgctcagatgcgtgtcgtgccgcagccagccgcgagcgcgccaacaaacgccacgccgaggagctggaggcggctaggtcgcaaatggcgctggaagtgcgtcccccgagcgaaattttggccatggtcgtcacagagctggaagcggcagcgagaattatcgcgatcgtggcggtgcccgcaggcatgacaaacatcgtaaatgccgcgtttcgtgtgccgtggccgcccaggacgtgtcagcgccgccaccacctgcaccgaatcggcagcagcgtcgcgcgtcgaaaaagcgcacaggcggcaagaagcgataagctgcacgaatacctgaaaaatgttgaacgccccgtgagcggtaactcacagggcgtcggctaacccccagtccaaacctgggagaaagcgctcaaaaatgactctagcggattcacgagacattgacacaccggcctggaaattttccgctgatctgttcgacacccatcccgagctcgcgctgcgatcacgtggctggacgagcgaagaccgccgcgaattcctcgctcacctgggcagagaaaatttccagggcagcaagacccgcgacttcgccagcgcttggatcaaagacccggacacggagaaacacagccgaagttataccgagttggttcaaaatcgcttgcccggtgccagtatgttgctctgacgcacgcgcagcacgcagccgtgcttgtcctggacattgatgtgccgagccaccaggccggcgggaaaatcgagcacgtaaaccccgaggtctacgcgattttggagcgctgggcacgcctggaaaaagcgccagcttggatcggcgtgaatccactgagcgggaaatgccagctcatctggctcattgatccggtgtatgccgcagcaggcatgagcagcccgaatatgcgcctgctggctgcaacgaccgaggaaatgacccgcgttttcggcgctgaccaggctttttcacataggctgagccgtggccactgcactctccgacgatcccagccgtaccgctggcatgcccagcacaatcgcgtggatcgcctagctgatcttatggaggttgctcgcatgatctcaggcacagaaaaacctaaaaaacgctatgagcaggagttttctagcggacgggcacgtatcgaagcggcaagaaaagccactgcggaagcaaaagcacttgccacgcttgaagcaagcctgccgagcgccgctgaagcgtctggagagctgatcgacggcgtccgtgtcctctggactgctccagggcgtgccgcccgtgatgagacggcttttcgccacgctttgactgtgggataccagttaaaagcggctggtgagcgcctaaaagacaccaagggtcatcgagcctacgagcgtgcctacaccgtcgctcaggcggtcggaggaggccgtgagcctgatctgccgccggactgtgaccgccagacggattggccgcgacgtgtgcgcggctacgtcgctaaaggccagccagtcgtccctgctcgtcagacagagacgcagagccagccgaggcgaaaagctctggccactatgggaagacgtggcggtaaaaaggccgcagaacgctggaaagacccaaacagtgagtacgcccgagcacagcgagaaaaactagctaagtccagtcaacgacaagctaggaaagctaaaggaaatcgcttgaccattgcaggttggtttatgactgttgagggagagactggctcgtggccgacaatcaatgaagctatgtctgaatttagcgtgtcacgtcagaccgtgaatagagcacttaaggtctgcgggcattgaacttccacgaggacgccgaaagcttcccagtaaatgtgccatctcgtaggcagaaaacggttcccccgtagggtctctctcttggcctcctttctaggtcgggctgattgctcttgaagctctctaggggggctcacaccataggcagataacgttccccaccggctcgcctcgtaagcgcacaaggactgctcccaaagatcttcaaagccactgccgcgactgccttcgcgaagccttgccccgcggaaatttcctccaccgagttcgtgcacacccctatgccaagcttctttcaccctaaattcgagagattggattcttaccgtggaaattcttcgcaaaaatcgtcccctgatcgcccttgcgacgttggcgtcggtgccgctggttgcgcttggcttgaccgacttgatcagcggccgc

SEQ ID NO: 16 - DNA, 7534 bp, Artificial Sequence
  Description of Artificial Sequence Synthetic nucleotide sequence
tcgatttaaatctcgagaggcctgacgtcgggcccggtaccacgcgtcatatgactagttcggacctagggatatcgtcgaccggcttaaagtttggctgccatgtgaatttttagcaccctcaacagttgagtgctggcactctcgggggtagagtgccaaataggttgtttgacacacagttgttcacccgcgacgacggctgtgctggaaacccacaaccggcacacacaaaatttttctcatggagggattcatcatgacttccaacttttcttccactgtcgctggtcttcctcgcatcggagcgaagcgtgaactgaagttcgcgctcgaaggctactggaatggatcaattgaaggtcgcgaacttgcgcagaccgcccgccaattggtcaacactgcatcggattctttgtctggattggattccgttccgtttgcaggacgttcctactacgacgcaatgctcgataccgccgctattttgggtgtgctgccggagcgttttgatgacatcgctgatcatgaaaacgatggtctcccactgtggattgaccgctactttggcgctgctcgcggtactgagaccctgcctgcacaggcaatgaccaagtggtttgataccaactaccactacctcgtgccggagttgtctgcggatacacgtttcgttttggatgcgtccgcgctgattgaggatctccgttgccagcaggttcgtggcgttaatgcccgccctgttctggttggtccactgactttcctttcccttgctcgcaccactgatggttccaatcctttggatcacctgcctgcactgtttgaggtctacgagcgcctcatcaagtctttcgatactgagtgggttcagatcgatgagcctgcgttggtcaccgatgttgctcctgaggttttggagcaggtccgcgctggttacaccactttggctaagcgcgatggcgtgtttgtcaatacttacttcggctctggcgatcaggcgctgaacactcttgcgggcatcggccttggcgcgattggcgttgacttggtcacccatggcgtcactgagcttgctgcgtggaagggtgaggagctgctggttgcgggcatcgttgatggtcgtaacatttggcgcaccgacctgtgtgctgctcttgcttccctgaagcgcctggcagctcgcggcccaatcgcagtgtctacctcttgttcactgctgcacgttccttacaccctcgaggctgagaacattgagcctgaggtccgcgactggcttgccttcggctcggagaagatcaccgaggtcaagctgcttgccgacgccctagccggcaacatcgacgcggctgcgttcgatgcggcgtccgcagcaattgcttctcgacgcacctccccacgcaccgcaccaatcacgcaggaactccctggccgtagccgtggatccttcgacactcgtgttacgctgcaggagaagtcactggagcttccagctctgccaaccaccaccattggttctttcccacagaccccatccattcgttctgctcgcgctcgtctgcgcaaggaatccatcactttggagcagtacgaagaggcaatgcgcgaagaaatcgatctggtcatcgccaagcaggaagaacttggtcttgatgtgttggttcacggtgagccagagcgcaacgacatggttcagtacttctctgaacttctcgacggtttcctctcaaccgccaacggctgggtccaaagctacggctcccgctgtgttcgtcctccagtgttgttcggaaacgtttcccgcccagcgccaatgactgtcaagtggttccagtacgcacagagcctgacccagaagcatgtcaagggaatgctcaccggtccagtcaccatccttgcatggtccttcgttcgcgatgatcagccgctggctaccactgctgaccaggttgcactggcactgcgcgatgaaattaacgatctcatcgaggctggcgcgaagatcatccaggtggatgagcctgcgattcgtgaactgttgccgctacgagacgtcgataagcctgcctacctgcagtggtccgtggactccttccgcctggcgactgccggcgcacccgacgacgtccaaatccacacccacatgtgctactccgagttcaacgaagtgatctcctcggtcatcgcgttggatgccgatgtcaccaccatcgaagcagcacgttccgacatgcaggtcctcgctgctctgaaatcttccggcttcgagctcggcgtcggacctggtgtgtgggatatccactccccgcgcgttccttccgcgcagaaagtggacggtctcctcgaggctgcactgcagtccgtggatcctcgccagctgtgggtcaacccagactgtggtctgaagacccgtggatggccagaagtggaagcttccctaaaggttctcgttgagtccgctaagcaggctcgtgagaaaatcggagcaactatctaatctagagttctgtgaaaaacaccgtggggcagtttctgcttcgcggtgttttttatttgtggggcactagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgcgcaaagtcccgcttcgtgaaaattttcgtgccgcgtgattttccgccaaaaactttaacgaacgttcgttataatggtgtcatgaccttcacgacgaagtactaaaattggcccgaatcatcagctatggatctctctgatgtcgcgctggagtccgacgcgctcgatgctgccgtcgatttaaaaacggtgatcggatttttccgagctctcgatacgacggacgcgccagcatcacgagactgggccagtgccgcgagcgacctagaaactctcgtggcggatcttgaggagctggctgacgagctgcgtgctcggccagcgccaggaggacgcacagtagtggaggatgcaatcagttgcgcctactgcggtggcctgattcctccccggcctgacccgcgaggacggcgcgcaaaatattgctcagatgcgtgtcgtgccgcagccagccgcgagcgcgccaacaaacgccacgccgaggagctggaggcggctaggtcgcaaatggcgctggaagtgcgtcccccgagcgaaattttggccatggtcgtcacagagctggaagcggcagcgagaattatcgcgatcgtggcggtgcccgcaggcatgacaaacatcgtaaatgccgcgtttcgtgtgccgtggccgcccaggacgtgtcagcgccgccaccacctgcaccgaatcggcagcagcgtcgcgcgtcgaaaaagcgcacaggcggcaagaagcgataagctgcacgaatacctgaaaaatgttgaacgccccgtgagcggtaactcacagggcgtcggctaacccccagtccaaacctgggagaaagcgctcaaaaatgactctagcggattcacgagacattgacacaccggcctggaaattttccgctgatctgttcgacacccatcccgagctcgcgctgcgatcacgtggctggacgagcgaagaccgccgcgaattcctcgctcacctgggcagagaaaatttccagggcagcaagacccgcgacttcgccagcgcttggatcaaagacccggacacggagaaacacagccgaagttataccgagttggttcaaaatcgcttgcccggtgccagtatgttgctctgacgcacgcgcagcacgcagccgtgcttgtcctggacattgatgtgccgagccaccaggccggcgggaaaatcgagcacgtaaaccccgaggtctacgcgattttggagcgctgggcacgcctggaaaaagcgccagcttggatcggcgtgaatccactgagcgggaaatgccagctcatctggctcattgatccggtgtatgccgcagcaggcatgagcagcccgaatatgcgcctgctggctgcaacgaccgaggaaatgacccgcgttttcggcgctgaccaggctttttcacataggctgagccgtggccactgcactctccgacgatcccagccgtaccgctggcatgcccagcacaatcgcgtggatcgcctagctgatcttatggaggttgctcgcatgatctcaggcacagaaaaacctaaaaaacgctatgagcaggagttttctagcggacgggcacgtatcgaagcggcaagaaaagccactgcggaagcaaaagcacttgccacgcttgaagcaagcctgccgagcgccgctgaagcgtctggagagctgatcgacggcgtccgtgtcctctggactgctccagggcgtgccgcccgtgatgagacggcttttcgccacgctttgactgtgggataccagttaaaagcggctggtgagcgcctaaaagacaccaagggtcatcgagcctacgagcgtgcctacaccgtcgctcaggcggtcggaggaggccgtgagcctgatctgccgccggactgtgaccgccagacggattggccgcgacgtgtgcgcggctacgtcgctaaaggccagccagtcgtccctgctcgtcagacagagacgcagagccagccgaggcgaaaagctctggccactatgggaagacgtggcggtaaaaaggccgcagaacgctggaaagacccaaacagtgagtacgcccgagcacagcgagaaaaactagctaagtccagtcaacgacaagctaggaaagctaaaggaaatcgcttgaccattgcaggttggtttatgactgttgagggagagactggctcgtggccgacaatcaatgaagctatgtctgaatttagcgtgtcacgtcagaccgtgaatagagcacttaaggtctgcgggcattgaacttccacgaggacgccgaaagcttcccagtaaatgtgccatctcgtaggcagaaaacggttcccccgtagggtctctctcttggcctcctttctaggtcgggctgattgctcttgaagctctctaggggggctcacaccataggcagataacgttccccaccggctcgcctcgtaagcgcacaaggactgctcccaaagatcttcaaagccactgccgcgactgccttcgcgaagccttgccccgcggaaatttcctccaccgagttcgtgcacacccctatgccaagcttctttcaccctaaattcgagagattggattcttaccgtggaaattcttcgcaaaaatcgtcccctgatcgcccttgcgacgttggcgtcggtgccgctggttgcgcttggcttgaccgacttgatcagcggccgc

SEQ ID NO: 17 - DNA, 7322 bp, Artificial Sequence
  Description of Artificial Sequence Synthetic nucleotide sequence
ggccgctcgatttaaatctcgagctctggagtgcgacaggtttgatgataaaaaattagcgcaagaagacaaaaatcaccttgcgctaatgctctgttacaggtcactaataccatctaagtagttgattcatagtgactgcatatgtaagtatttccttagataacaattgattgaatgtatgcaaataaatgcatacaccataggtgtggtttaatttgatgccctttttcagggctggaatgtgtaagagcggggttatttatgctgttgtttttttgttactcgggaagggctttacctcttccgcataaacgcttccatcagcgtttatagttaaaaaaatctttcggggggatggggagtaagcttgtgttatccgctcgggcccaatccgcaagctccaccgactcgttggcgtgcgactctagataaatatcaagcagctggccgccaataacctcagtacgcatgccacgccaagcatccctcgtgcgggccaatgcctctgcactcaaaccggaatcctgcagcatgtcttctgcccacaccaatgccatatcgccagccaaaatcgagactgaaacgccaaagtgctcgggatcgccttcgaaattattggcgcggtgatcagcttccacagcccggtgaactgtgggggctccgcgccgggtatcagaagaatcgataatatcgtcatgaatcaaggcacaagcctggatgaattcgagactcgctgcggcgtcaaggacggactcaagtttttcagaagaattcttatggccttgcgccgccaggaaaccagcccacgcataaagaggacggattcgctttcctccattgagcacgaaactgcgaagatgggccacagcatctgtgacaggagcgccgatatcagcaattgttagctcttgagcatcgaggaactgcgtcaaacgatctcgcacgacctccggaaatttgtcgaggtcaaggtcatgggcatcgaaactgctcaaggagacgtccttcaatcgaatagggggatgcgggctgaattttggtggaggtgaataaatgccagaggcagtcccaacaaaacactctcatcacactaagatacccgtcgactcatacgttaaatctatcaccgcaagggataaatatctaacaccgtgcgtgttgactattttacctctggcggtgataatggttgcatgtactaaggaggattaattaatgtccctaacgaacatcccagcctcatctcaatgggcaattagcgacgttttgaagcgtccttcacccggccgagtacctttttctgtcgagtttatgccaccccgcgacgatgcagctgaagagcgtctttaccgcgcagcagaggtcttccatgacctcggtgcatcgtttgtctccgtgacttatggtgctggcggatcaacccgtgagagaacctcacgtattgctcgacgattagcgaaacaaccgttgaccactctggtgcacctgaccctggttaaccacactcgcgaagagatgaaggcaattcttcgggaatacctagagctgggattaacaaacctgttggcgcttcgaggagatccgcctggagacccattaggcgattgggtgagcaccgatggaggactgaactatgcctctgagctcatcgatcttattaagtccactcctgagttccgggaattcgacctcggtatcgcctccttccccgaagggcatttccgggcgaaaactctagaagaagacaccaaatacactctggcgaagctgcgtggaggggcagagtactccatcacgcagatgttctttgatgtggaagactacctgcgacttcgtgatcgccggatcctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttggtacgggatttaaatgatccgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggcc

SEQ ID NO: 18 - DNA, 7520 bp, Artificial Sequence
  Description of Artificial Sequence Synthetic nucleotide sequence
ctggagtgcgacaggtttgatgataaaaaattagcgcaagaagacaaaaatcaccttgcgctaatgctctgttacaggtcactaataccatctaagtagttgattcatagtgactgcatatgtaagtatttccttagataacaattgattgaatgtatgcaaataaatgcatacaccataggtgtggtttaatttgatgccctttttcagggctggaatgtgtaagagcggggttatttatgctgttgtttttttgttactcgggaagggctttacctcttccgcataaacgcttccatcagcgtttatagttaaaaaaatctttcggggggatggggagtaagcttgtgttatccgctcgggcccggtaccacgcgtgagttctttgagttcctgtggggtgaacttgacctgtgctgggccacgacgtccgaaaacgtgcacttcagtggccttgttttctttgagggagtcgtagacgttgtcggaaatttcggtgactttgagctcgtcgcctgtcttagccaggatgcgggctacgtcgaggccgacgttaccaacgccgataacagcgacggactgtgcagacagatcccaggagcgctcgaagcgtgggttgccgtcgtagaagccaacgaactcgccggcaccgaaggagccttctgcttcaattccggggatgttgaggtcgcggtctgcaactgcgccggtggagaacacgactgcatcgtagtagtcgcggagttcttcgacggtgatgtctttgccgatttcaatgttaccgagcaggcgcaggcgtggcttgtccaacacgttgtgcagggacttaacgatgcccttgatgcgtgggtggtctggagcaacgccgtaacggatgagtccgaacggtgcaggcatttgctcgaaaaggtcaacgaacacttcgcgctcttcattgcggatgaggaggtcggatgcgtaaatgccagcagggccagctccgatgacggctacgcgcaggggagttgtcataatattacacctccttagataacaattgattgaatgtatgcaaataaatgcccttcttcagggcttaatttttaagagcgtcaccttcatggtggtcagtgcgtcctgctgatgtgctcagtatcaccgccagtggtatttatgtcaacaccgccagagataatttatcaccgcagatggttatctgtatgttttttatatgaatttattttttgcaggggggcattgtttggtaggtgagagatcaattctgcgtcgactcatacgttaaatctatcaccgcaagggataaatatctaacaccgtgcgtgttgactattttacctctggcggtgataatggttgcatgtactaaggaggattaattaatgacaacaaccaccggaagtgcccggccagcacgtgccgccaggaagcctaagcccgaaggccaatggaaaatcgacggcaccgagccgcttaaccatgccgaggaaattaagcaagaagaacccgcttttgctgtcaagcagcgggtcattgatatttactccaagcagggtttttcttccattgcaccggatgacattgccccacgctttaagtggttgggcatttacacccagcgtaagcaggatctgggcggtgaactgaccggtcagcttcctgatgatgagctgcaggatgagtacttcatgatgcgtgtgcgttttgatggcggactggcttcccctgagcgcctgcgtgccgtgggtgaaatttctagggattatgctcgttccaccgcggacttcaccgaccgccagaacattcagctgcactggattcgtattgaagatgtgcctgcgatctgggagaagctagaaaccgtcggactgtccaccatgcttggttgcggtgacgttccacgtgttatcttgggctccccagtttctggcgtagctgctgaagagctgatcgatgccaccccggctatcgatgcgattcgtgagcgctacctagacaaggaagagttccacaaccttcctcgtaaggatcctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttggtacgggatttaaatgatccgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatctcgagct

SEQ ID NO: 19 - DNA, 5697 bp, Artificial Sequence
  Description of Artificial Sequence Synthetic nucleotide sequence
tcgaggcgtcttccggtgtcatggttgaaccgaattccagcacaatattttccggtttaaagcaatcgatcacatagtcgattttgtccaaccactgaaaacctgcaaggaccacccaatcccctgcagcatgttcagcaaccattggcagcggcggatagcgaacttcccccttttctcccgttgccattttcgcgtcactgatcaggtgactgagctttttgtagccttccggatttttacacaagactgtcaacacgccttcttgcagactcagctccgcaccataaacggtatgcattccagcttccgcggcagcttccgcaaatctcactgcaccataaaaaccatccctatccatgactgatagagcaacaagtcctaactttttggcctgcacaaccacatcagacggatccgatgcgccagtgagaaagttataactgctggtggcatgcagctcggcaaaaggaaccgacgcttccccctgcatggcagatgaaggcgcctgcgcatccggctcatgcagcaccggacgcagagattcgacctttttacctgagaggattctttccaatttggaccacgataatggcctgccgttaaagcttcccccgccattccattccataatgataggatacatttttagaacaaattttccaataagttttccacgccagccggagaaggaaatagaccaagctgtacagatcgacgcgtcctggctgagtacaacgtcggctccggcgcagacctcaccccagttggctccagcgaaatcgtgccactggcactattctggaaggaccacgactccatcgacggcattgacggcgagtccgttgccatccctaacgatccttccaaccagggccgcgccatcaacgttctcgttcaggcaggtctggtcaccctgaagaccccaggtctggtcaccccagctccagtcgatatcgacgaggcagcttccaaggtttccgtcatcccagtcgacgcagctcaggcaccaaccgcttaccaggagggtcgcccagcgatcatcaacaactccttccttgaccgcgcaggcatcgatccaaacctcgcggtcttcgaagatgatcctgagtctgaagaagcagagccatacatcaacgtcttcgtcaccaaggctgaggacaaggacgatgccaacatcgcccgcctcgttgagctgtggcacgacccagaggttctggctgcagtagaccgcgactctgagggcacctccgtcccagttgatcgtccaggagctgaccttcaggaaatccttgatcgccttgaggctgatcaggaaaacgcataatctcttttgagttctttgcatacccatgtgcagatttctttgcacaatcacagcctgaaaatcagactgtgaacttcaaacgcatatgactagttcggacctagggatatcgtcgacatcgatgctcttctgcgttaattaacaattgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 20 - DNA, 7767 bp, Artificial Sequence
  Description of Artificial Sequence Synthetic nucleotide sequence
tcgatttaaatctcgagctctggagtgcgacaggtttgatgataaaaaattagcgcaagaagacaaaaatcaccttgcgctaatgctctgttacaggtcactaataccatctaagtagttgattcatagtgactgcatatgtaagtatttccttagataacaattgattgaatgtatgcaaataaatgcatacaccataggtgtggtttaatttgatgccctttttcagggctggaatgtgtaagagcggggttatttatgctgttgtttttttgttactcgggaagggctttacctcttccgcataaacgcttccatcagcgtttatagttaaaaaaatctttcggggggatggggagtaagcttgtgttatccgctcgggcccggtaccacgcgtcatatgactagttcggacctagggatatcgtcgactcatacgttaaatctatcaccgcaagggataaatatctaacaccgtgcgtgttgactattttacctctggcggtgataatggttgcatgtactaaggaggattaattaatgtccctaacgaacatcccagcctcatctcaatgggcaattagcgacgttttgaagcgtccttcacccggccgagtacctttttctgtcgagtttatgccaccccgcgacgatgcagctgaagagcgtctttaccgcgcagcagaggtcttccatgacctcggtgcatcgtttgtctccgtgacttatggtgctggcggatcaacccgtgagagaacctcacgtattgctcgacgattagcgaaacaaccgttgaccactctggtgcacctgaccctggttaaccacactcgcgaagagatgaaggcaattcttcgggaatacctagagctgggattaacaaacctgttggcgcttcgaggagatccgcctggagacccattaggcgattgggtgagcaccgatggaggactgaactatgcctctgagctcatcgatcttattaagtccactcctgagttccgggaattcgacctcggtatcgcctccttccccgaagggcatttccgggcgaaaactctagaagaagacaccaaatacactctggcgaagctgcgtggaggggcagagtactccatcacgcagatgttctttgatgtggaagactacctgcgacttcgtgatcgccttgtcgctgcagaccccattcatggtgcgaagccaatcattcctggcatcatgcccattacgagcctgcggtctgtgcgtcgacaggtcgaactctctggtgctcaattgccgagccaactagaagaatcacttgttcgagctgcaaacggcaatgaagaagcgaacaaagacgagatccgcaaggtgggcattgaatattccaccaatatggcagagcgactcattgccgaaggtgcggaagatctgcacttcatgacgcttaacttcacccgtgcaacccaagaagtgttgtacaaccttggcatggcgcctgcttggggagcagagcacggccaagacgcggtgcgttaaggatcctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttggtacgggatttaaatgatccgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgcgcaaagtcccgcttcgtgaaaattttcgtgccgcgtgattttccgccaaaaactttaacgaacgttcgttataatggtgtcatgaccttcacgacgaagtactaaaattggcccgaatcatcagctatggatctctctgatgtcgcgctggagtccgacgcgctcgatgctgccgtcgatttaaaaacggtgatcggatttttccgagctctcgatacgacggacgcgccagcatcacgagactgggccagtgccgcgagcgacctagaaactctcgtggcggatcttgaggagctggctgacgagctgcgtgctcggccagcgccaggaggacgcacagtagtggaggatgcaatcagttgcgcctactgcggtggcctgattcctccccggcctgacccgcgaggacggcgcgcaaaatattgctcagatgcgtgtcgtgccgcagccagccgcgagcgcgccaacaaacgccacgccgaggagctggaggcggctaggtcgcaaatggcgctggaagtgcgtcccccgagcgaaattttggccatggtcgtcacagagctggaagcggcagcgagaattatcgcgatcgtggcggtgcccgcaggcatgacaaacatcgtaaatgccgcgtttcgtgtgccgtggccgcccaggacgtgtcagcgccgccaccacctgcaccgaatcggcagcagcgtcgcgcgtcgaaaaagcgcacaggcggcaagaagcgataagctgcacgaatacctgaaaaatgttgaacgccccgtgagcggtaactcacagggcgtcggctaacccccagtccaaacctgggagaaagcgctcaaaaatgactctagcggattcacgagacattgacacaccggcctggaaattttccgctgatctgttcgacacccatcccgagctcgcgctgcgatcacgtggctggacgagcgaagaccgccgcgaattcctcgctcacctgggcagagaaaatttccagggcagcaagacccgcgacttcgccagcgcttggatcaaagacccggacacggagaaacacagccgaagttataccgagttggttcaaaatcgcttgcccggtgccagtatgttgctctgacgcacgcgcagcacgcagccgtgcttgtcctggacattgatgtgccgagccaccaggccggcgggaaaatcgagcacgtaaaccccgaggtctacgcgattttggagcgctgggcacgcctggaaaaagcgccagcttggatcggcgtgaatccactgagcgggaaatgccagctcatctggctcattgatccggtgtatgccgcagcaggcatgagcagcccgaatatgcgcctgctggctgcaacgaccgaggaaatgacccgcgttttcggcgctgaccaggctttttcacataggctgagccgtggccactgcactctccgacgatcccagccgtaccgctggcatgcccagcacaatcgcgtggatcgcctagctgatcttatggaggttgctcgcatgatctcaggcacagaaaaacctaaaaaacgctatgagcaggagttttctagcggacgggcacgtatcgaagcggcaagaaaagccactgcggaagcaaaagcacttgccacgcttgaagcaagcctgccgagcgccgctgaagcgtctggagagctgatcgacggcgtccgtgtcctctggactgctccagggcgtgccgcccgtgatgagacggcttttcgccacgctttgactgtgggataccagttaaaagcggctggtgagcgcctaaaagacaccaagggtcatcgagcctacgagcgtgcctacaccgtcgctcaggcggtcggaggaggccgtgagcctgatctgccgccggactgtgaccgccagacggattggccgcgacgtgtgcgcggctacgtcgctaaaggccagccagtcgtccctgctcgtcagacagagacgcagagccagccgaggcgaaaagctctggccactatgggaagacgtggcggtaaaaaggccgcagaacgctggaaagacccaaacagtgagtacgcccgagcacagcgagaaaaactagctaagtccagtcaacgacaagctaggaaagctaaaggaaatcgcttgaccattgcaggttggtttatgactgttgagggagagactggctcgtggccgacaatcaatgaagctatgtctgaatttagcgtgtcacgtcagaccgtgaatagagcacttaaggtctgcgggcattgaacttccacgaggacgccgaaagcttcccagtaaatgtgccatctcgtaggcagaaaacggttcccccgtagggtctctctcttggcctcctttctaggtcgggctgattgctcttgaagctctctaggggggctcacaccataggcagataacgttccccaccggctcgcctcgtaagcgcacaaggactgctcccaaagatcttcaaagccactgccgcgactgccttcgcgaagccttgccccgcggaaatttcctccaccgagttcgtgcacacccctatgccaagcttctttcaccctaaattcgagagattggattcttaccgtggaaattcttcgcaaaaatcgtcccctgatcgcccttgcgacgttggcgtcggtgccgctggttgcgcttggcttgaccgacttgatcagcggccgc

(19) United States
(12) Patent Application Publication
(10) Pub. No.: US 2009/0298136 A1
(43) Pub. Date: Dec. 3, 2009

(54) Methionine producing recombinant microorganisms

(75) Inventors: Oskar Zelder, Speyer (DE); Stefan Haefner, Speyer (DE); Corinna Klopprogge, Mannheim (DE); Hartwig Schroder, Nussloch (DE); Andrea Herold, Ketsch (DE); Thomas A. Patterson, North Attleboro, MA (US); Theron Hermann, Kinnelon, NJ (US); R. Rogers Yocum, Lexington, MA (US); Mark K. William, Revere, MA (US); Janice G. Pero, Lexington, MA (US)

Correspondence Address: LAHIVE & COCKFIELD, LLP;FLOOR 30, SUITE 3000, ONE POST OFFICE SQUARE, BOSTON, MA, 02109, US

(73) Assignee: BASF AG, Ludwigshafen (DE)

(21) Appl. No.: 11/988,962

(22) PCT Filed: Jul. 18, 2006
(86) PCT No.: PCT/US2006/028439

§ 371 Date: Jun. 9, 2009

Related U.S. Application Data
(60) Provisional application No. 60/700,699, filed on Jul. 18, 2005.
(60) Provisional application No. 60/714,042, filed on Sep. 1, 2005.

(51) Int. Cl. C12P 13/12; C12N 1/21

(52) U.S. Cl. 435/113; 435/252.31; 435/252.32; 435/252.35

(57) ABSTRACT

This invention relates to methionine producing recombinant microorganisms. Specifically, this invention relates to recombinant strains of Corynebacterium that produce increased levels of methionine compared to their wild-type counterparts and further to methods of generating such microorganisms.

### RELATED APPLICATIONS

This application claims the benefit of priority to U.S. Provisional Patent Application No. 60/700,699, filed on Jul. 18, 2005, and U.S. Provisional Patent Application No. 60/714,042, filed on Sep. 1, 2005, both entitled “Methionine Producing Recombinant Microorganism,” the entire contents of each of which are incorporated by reference herein.

Additionally, this application is related to U.S. Provisional Patent Application No. 60/700,698, filed on Jul. 18, 2005, and U.S. Provisional Patent Application No. 60/713,907, filed on Sep. 1, 2005, both entitled “Use of Dimethyl Disulfide for Methionine Production in Microrganisms,” the entire contents of each of which are incorporated by reference herein.

This application is also related to U.S. Provisional Patent Application No. 60/700,557, filed Jul. 18, 2005, and U.S. Provisional Patent Application No. 60/713,905, filed Sep. 1, 2005, both entitled “Use of a Bacillus MetI Gene to Improve Methionine Production in Microorganisms,” the entire contents of each of which are incorporated by reference herein.

The entire contents of each of these patent applications are hereby expressly incorporated herein by reference including without limitation the specification, claims, and abstract, as well as any figures, tables, or drawings thereof.

### BACKGROUND

Methionine is an amino acid used in many different industries including, but not limited to, animal feed, pharmaceuticals, food additives, cosmetics and dietary supplements. Methionine can be produced on a large scale by many different methods. For example, methionine can be produced chemically by first reacting methyhmercaptan with acrolein producing the intermediate 3-methylmercaptopropionaldehyde (MMP). Further processing involves reacting MMP with hydrogen cyanide to form 5-(2-methylthioethyl) hydantoin, which is then hydrolyzed using caustics such as NaOH together with Na₂CO₃, NH₃ and CO₂. Subsequently, sodium DL-methionine is neutralized with sulfuric acid and Na₂CO₃ to yield D, L-methionine, Na₂SO₄, and CO₂. This process yields a large excess of unused compounds in comparison to the amount of methionine which poses an economic and ecological challenge.

Additionally, fermentation of microorganisms could potentially also be used for production of methionine on a large scale, for example, by cultivating microorganisms with nutrients including, but not limited to, carbohydrate sources, e.g., sugars, such as glucose, fructose, or sucrose, hydrolyzed starch, nitrogen sources, e.g., ammonia, and sulfur sources e.g., sulfate and/or thiosulfate, together with other necessary or supplemental media components. This process would yield L-methionine and biomass as a byproduct with no toxic dangerous, flammable, unstable, noxious starting materials.

However, the titer and yield of methionine produced using the existing processes are too low to be commercially viable. Therefore, there is a need to find improved methods of methionine production that avoid the production of toxic chemicals and harmful byproducts, while being commercially significant.

It has been reported that a high level of production of certain amino acids can be obtained by altering expression of as few as three or even fewer genes and/or proteins encoded by them. For example, a strain that produces 80 g/l of lysine can be constructed simply by altering the expression of aspartokinase, pyruvate carboxylase and homoserine-dehydrogenase (Ohnishi, J. et al., Appl. Microbiol. Biotechnol. 58(2):217-223 (2002)).

It has been reported that altering expression of the following genes alone or in combination with other genes in bacteria leads to methionine production: metF (See, WO/087386A2, WO 04/024931A2 and U.S. Publication No. 2002049305); metH (See, WO 04/024933A2 and US Publication No. 2002/0048793); metA (See, WO/024932 A2); met K (WO 03/100072 A2); sahH (See EP 1507008); metY (See U.S. Publication No. 20050064551); met R and/or met Z (See U.S. Publication No. 2002/0102664); metE (U.S. Publication No. 20020110877); metD (See U.S. Publication No. 20050074802), cysQ (See WO 02/42466A2); cysD, cysN, cysK, cysE and cysH (See WO 02/0086373); and metZ, metC and rxa 00657 (See WO 01/66573). It has also been reported that generation of analogous resistant strains; such as for example, ethionine-resistant strains of amino acid producing bacteria, can lead to production of methionine. (Kumar and Gomes, Biotechnology advances 23: 41-61 (2005)).

However, because methionine biosynthesis involves incorporation of a reduced sulfur atom and is considered to be more complex than the biosynthesis of other amino acids, it is not clear which combination of altered genes and/or use of resistant strains would be required for the production of commercially attractive levels of methionine.

### SUMMARY

The present invention features new and improved methods for increasing production of methionine. In particular, the invention is based, at least in part, on the discovery that alteration of certain genes, for example, by genetic engineering and classical genetics in microorganisms, e.g., Cornyebacterium glutamicum, provides an increased production of methionine.

The present invention further relates to recombinant microorganisms that produce increased levels of methionine relative to methionine produced by their wild-type counterparts, methods of producing such microorganisms, and methods for producing methionine that use such microorganisms. In some embodiments, certain combinations of altered genes lead to increased methionine production which is substantially higher than any titer that has previously been reported, for example, at least 15 g/l, or at least 16 g/l, or at least 17 g/l or higher.

In some embodiments, recombinant microorganisms described herein include genetic alterations in each of any two or more, or three or more, or four or more, or five or more, or six or more, or seven or more, or eight or more genes chosen from as ask^{fbr}, hom^{fbr}, metX, metY, metB, metH, metE, metF and zwf; where the genetic alterations lead to overexpression of the genes, thereby leading to increased methionine production by the microorganism relative to methionine production in absence of the genetic alterations in each of the two or more, or three or more, or four or more, or five or more, or six or more, or seven or more, or eight or more genes. In some embodiments, recombinant microorganisms have genetic alterations in each of at least five genes chosen from ask^{fbr}, hom^{fbr}, metX, metY, metB, metH, metE, metF and zwf, where the genetic alterations lead to overexpression of the at least five genes, thereby resulting in ah increased methionine production by the microorganism relative to the methionine produced in the absence of the genetic alterations in each of the at least five genes. Also described herein are recombinant microorganisms including genetic alterations in each of any six genes, or each of any seven genes, or each of any eight genes chosen from ask^{fbr}, hom^{fbr}, metX, metY, metB, metH, metE, metF and zwf, where the genetic alterations lead to overexpression of the genes, thereby leading to increased methionine production by the microorganism relative to methionine production in absence of the genetic alterations in each of the any six genes, or any seven genes, or any eight genes. Recombinant microorganisms may also include genetic alterations in all of the nine genes ask^{fbr}, hom^{fbr}, metX, metY, metB, metH, metE, metF and zwf, where the genetic alterations lead to overexpression of the nine genes, thereby leading to increased methionine production by the microorganism relative to methionine production in absence of the genetic alterations in each of the nine genes.

As described herein, overexpression can be achieved by various means, including but not limited to, for example, increasing transcription/translation of a gene by, for example, introducing promoter and/or enhancer sequences upstream of the gene, substituting the promoter with a heterologous promoter which increases expression of the gene or leads to constitutive expression of the gene, increasing copy number of the gene, using episomal plasmids, or by modifying the gene sequence, and any combination of such methods, such that the enzyme(s) encoded by the gene has increased activity or increased resistance to inhibition by one or more inhibitory compounds relative to its wild-type counterpart. Additionally, overexpression can also be achieved by, for example, deleting or mutating the gene for a transcriptional factor which normally represses expression of the gene desired to be overexpressed.

In some embodiments, recombinant microorganisms described herein include genetic alterations in each of any two genes chosen from mcbR, hsk, metQ, metK and pepCK, where the genetic alterations decrease expression of the any two genes and/or an activity of the protein encoded by the any two genes (e.g., enzymatic activity) thereby leading to increased methionine production by the microorganism relative to methionine production in absence of the genetic alterations in each of the any two genes. In yet other embodiments, recombinant microorganisms encompassed by the present invention include genetic alterations in each of any three genes, or any four genes, or all five genes chosen from mcbR, hsk, metQ, metK and pepCK, where the genetic alterations decrease the expression of the genes and/or an activity of proteins encoded by the genes, thereby leading to increased methionine production by the microorganism relative to methionine production in absence of the genetic alterations in each of the any three genes, or four genes, or all five genes. As used herein, a decrease in expression of a gene can be achieved by many different means, including but not limited to, for example, mutating the promoter of the gene, replacing the promoter of the gene with a heterologous promoter which lowers the expression of the gene, or by modifying a gene sequence such that it encodes a protein or enzyme(s) with a lower activity than its wild-type counterpart. In certain instances, decrease in expression is achieved by deleting or mutating a gene sequence such that lower level of a protein or enzyme is produced or no protein or enzyme is produced. Additionally, a decrease in expression of a gene can be achieved by, for example, increasing the expression of a transcriptional repressor for the gene.

In some embodiments, recombinant microorganisms encompassed by the present invention include genetic alterations in each of any two genes, or any three genes, or any five genes, or any six genes, or any seven genes, or any eight genes, or all nine genes chosen from ask^{fbr}, hom^{fbr}, metX, metY, metB, metH, metE, metF and zwf, where the genetic alterations lead to overexpression of each of the any two genes, or any three genes, or any four genes, or any five genes, or the any six genes, or the any seven genes, or the any eight genes, or the nine genes, in combination with genetic alterations in each of any one gene, or any two genes, or any three genes, or any four genes, or five genes chosen from mcbR, hsk, metQ, metK and pepCK, where the genetic alterations decrease expression of the any one gene, or the any two genes, or the any three gene, or the any four genes, or the five genes, where the combination results in increased methionine production by the microorganism relative to methionine production in absence of the combination. In some embodiments, recombinant microorganisms include genetic alterations in each of at least five genes chosen from ask^{fbr}, hom^{fbr}, metX, metY, metB, metH, metE, metF and zwf, where the genetic alterations lead to overexpression of each of the at least five genes in combination with genetic alterations in at least one gene chosen from mcbR, hsk, metQ, metK and pepCK, thereby resulting in decreased expression of the at least one gene, wherein the microorganism produces increased level of methionine relative to the methionine produced in absence of the combination.

For example, in some embodiments, recombinant microorganisms described herein include genetic alterations in each gene chosen from a group consisting of ask^{fbr}, hom^{fbr}, metH, and ask^{fbr}, hom^{fbr} metE, thereby resulting in overexpression of the each gene, in combination with genetic alterations in each of mcbR and hsk, thereby resulting in decreased expression of mcbR and hsk, wherein the microorganism produces increased level of methionine relative to the methionine produced in absence of the combination. In yet other embodiments described herein, recombinant microorganisms include genetic alterations in each of at least six genes chosen from the group consisting of ask^{fbr}, hom^{fbr}, metX (also called metA), metY (also called metZ), metF, metH, metE and ask^{fbr}, hom^{fb}, metX, metY, metF and metE, thereby resulting in overexpression of the at least six genes in combination with genetic alterations in each of mcbR and hsk, thereby resulting in decreased expression of mcbR and hsk, wherein the microorganism produces increased level of methionine relative to the methionine produced in the absence of the combination.

Recombinant microorganisms described herein may further include genetic alterations resulting in overexpression of one or more genes in the cysteine biosynthetic pathway. For example, in certain embodiments, recombinant microorganisms described herein include genetic alterations in each of two or more, or three or more, or four or more, or five or more, or six or more, or seven or more, or eight or more, or nine or more, or ten or more, or eleven or more, or twelve or more, or thirteen or more, or fourteen or more, or fifteen or more, or sixteen or more, or seventeen or more, or eighteen or more, or nineteen or more, or twenty or more, or twenty one or more, or twenty two or more, or twenty three or more, or twenty four or more, or twenty five or more, or twenty six or more, or twenty seven or more, or twenty eight or more, or twenty nine or more, or thirty or more, or thirty one or more, or thirty two or more, or thirty three or more, or thirty four, genes chosen from ask^{fbr}, hom^{fbr}, metX (also referred to as metA), metY (also referred to as metZ), metB, metK, metQ, metH, metE, metF, metC, zwf, frpA1, asd, cysE, cysK, cysN, cysD, cysH, cysI, cysC, cysX, cysM, cysA, cysQ cysG, cysZ, cysJ, cysY, hsk, mcbR, pyc, pepCK and ilvA, thereby resulting in increased production of methionine relative to that produced in absence of the genetic alterations.

In some embodiments, recombinant microorganisms described herein include genetic alterations in each of at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty one, or at least twenty two, or at least twenty three, or at least twenty four, or at least twenty five, or twenty six genes chosen from ask^{fbr}, hom^{fbr}, metX (also referred to as metA), metY (also referred to as metZ), metB, metH, metE, metF, metC, zwf, frpA, asd, cysE, cysK, cysN, cysA, cysD, cysH, cysI, cysC, cysX, cysG, cysM, cysZ, cysJ, and pyc, where the at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty one, or at least twenty two, or at least twenty three, or at least twenty four, or at least twenty five, or twenty six genes are overexpressed, thereby resulting in increased production of methionine relative to the methionine production in the absence of the genetic alterations. For example, in some embodiments, recombinant microorganisms include genetic alterations in each of at least eight genes chosen from ask^{fbr}, hom^{fbr}, metX (also referred to as metA), metY (also referred to as metZ), metB, metH, metE, metF, metC, zwf frpA, asd, cysE, cysK, cysN, cysA, cysD, cysH, cysI, cysI, cysC, cysG, cysM, cysZ, cysJ, and pyc, where the genetic alterations lead to overexpression of the at least eight genes, thereby resulting in increased production of methionine relative to methionine produced in absence of the genetic alterations.

In some embodiments recombinant microorganisms include genetic alterations in each of at least five genes chosen from ask^{fbr}, hom^{fbr}, metX, metY, metB, metH, metE, metF and zwf, where the genetic alterations lead to overexpression of each of the at least five genes in combination with at least six genes chosen from cysE, cysK, cysN, cysA, cysD, cysH, cysI, cysC, cysX, cysG, cysM, cysZ, and cysJ, where the genetic alterations result in overexpression of the at least six genes, where the combination results in an increased production of methionine by the microorganism relative to the production in absence of the combination.

In yet other embodiments, recombinant microorganisms include genetic alterations in each of at least two genes chosen from metK, metQ, cysQ, cysY, hsk, mcbR, pepCK and ilvA, where the expression of at least two genes is decreased, thereby resulting in increased production of methionine relative to the methionine production in the absence of the genetic alterations.

In some embodiments, recombinant microorganisms include deregulation of at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty one, or at least twenty two, or at least twenty three, or at least twenty four, or at least twenty five proteins chosen from: Aspartate kinase, Homoserine Dehydrogenase, Homoserine Acetyltransferase, Homoserine Succinyltransferase, Cystathionine γ-synthase, Cystathionine β-lyase, O-Acetylhomoserine sulfhydralase, O-Succinylhomoserine sulfhydralase, Vitamin 12-dependent methionine synthase, Vitamin B12-independent methionine synthase, N5,10-methylene-tetrahydrofolate reductase, Sulfate adenylyltransferase subunit 1, Sulfate adenylyltransferase subunit 2, APS kinase, APS reductase, Phosphoadenosine phosphosulfate reductase, NADP-ferredoxin reductase, Sulfite reductase subunit 1, Sulfite reductase subunit 2, Sulfate transporter, Serine O-acetyltransferase, O-acetyl serine (thiol)-lyase A, Uroporphyrinogen III synthase, Glucose-6-phosphate dehydrogenase, Pyruvate carboxylase, and Aspartate semialdehyde dehydrogenase, where the deregulation includes overexpression of the proteins, thereby resulting in production of methionine in an amount of at least 8 g/l under suitable conditions. In some embodiments, recombinant microorganisms include deregulation of at least five proteins described herein, thereby resulting in production of methionine in an amount of at least 8 g/l under suitable conditions. In yet other embodiments, recombinant microorganisms include deregulation of at least eight proteins described herein, thereby resulting in production of methionine in an amount of at least 16 g/l under suitable conditions. Suitable conditions, as described herein, are conditions which result in an increased production of methionine by the recombinant microorganisms described herein.

In some embodiments described herein, recombinant microorganisms produce methionine in an amount of at least 8 g/l, or at least 9 g/l, or at least 10 g/l, or at least 11 g/l, or at least 12 g/l, or at 13 g/l, or at least 14 g/l, or at least 15 g/l, or at least 16 g/l under suitable conditions. In some embodiments, recombinant microorganisms produce methionine in an amount of at least 8 g/l. In other embodiments, recombinant microorganisms described herein produce methionine in an amount of at least 16 g/l.

In some embodiments, recombinant microorganisms include genetic alterations in each of at least five genes chosen from ask^{fbr}, hom^{fbr}, metX, metY, metB, metH, metE, metF and zwf, where the genetic alterations lead to overexpression of each of the at least five genes in combination with genetic alterations in at least one gene chosen from metK, metQ, hsk, mcbR and pepCK, thereby resulting in decreased expression of the at least one gene, wherein the combination results in methionine production of at least 8 g/l by the microorganism under suitable conditions for example, as described herein.

In one exemplary embodiment, a recombinant microorganism encompassed by the present invention comprises genetic alterations in each of eight genes chosen from ask, hom, metX, metY, metE, metH, metF and mcbR, wherein the titer of methionine produced by the microorganism under suitable conditions is at least 16 g/l.

In some embodiments, overexpression of genes includes constitutive expression of the gene and/or a polypeptide encoded by the gene.

In some embodiments, recombinant microorganisms described herein are ethionine-resistant. Therefore, also encompassed by the present invention are ethionine-resistant recombinant microorganisms including one of the many combinations of genetic alterations, as described herein, where the combination of the ethionine resistance and the genetic alterations results in increased methionine production relative to methionine produced in the absence of the combination. In some embodiments, ethionine-resistant microorganisms including a combination of genetic alterations, as described herein, produce methionine in an amount of at least 8 g/l, or at least 9 g/l, or at least 10 g/l, or at least 11 g/l, or at least 12 g/l, or at least 13 g/l, or at least 14 g/l, or at least 15 g/l, or at least 16 g/l, or at least 17 g/l, or at least 18 g/l, or at least 19 g/l, or at least 20 g/l in a fermentation process.

In some embodiments described herein, recombinant microorganisms include a combination of: (1) genetic alterations in, each of at least six genes chosen from ask^{fbr}, hom^{fbr}, metX (also referred to as metA), metY (also referred to as metZ), metH, metF and ask^{fbr}, hom^{fbr}, metX (also referred to as metA), metY (also referred to as metZ), metH, metF and metE, thereby resulting in overexpression of each of the at least six genes; (2) genetic alterations in each of mcbR and hsk, thereby resulting in decreased expression of mcbR and hsk; and (3) an ethionine-resistant mutation; where the microorganism produces at least 16 g/l methionine under suitable conditions.

This invention further relates to methods of genetically engineering microorganisms that produce methionine at increased or enhanced levels. In some embodiments, the present invention provides vectors which may be introduced into microorganisms for making the various genetic alterations encompassed by this invention. Such genetic alterations may either increase expression of a gene or decrease expression of a gene. In some embodiments, vectors are used to introduce promoter and/or enhancer sequences upstream of a gene, thereby to increase expression of the gene.

Recombinant microorganisms described herein may either be Gram positive or Gram negative. In some embodiments, recombinant microorganisms belong to a genus chosen from Bacillus, Cornyebacterium, Lactobacillus, Lactococci and Streptomyces. In some embodiments, recombinant microorganisms described herein belong to genus Cornyebacterium, for example, a Cornyebacterium glutamicum strain.

In some embodiments, a method of producing methionine includes culturing a Cornyebacterium strain including genetic alterations in each of at least two, or at least three, or at least four, or at least five, or at least six, or at least seven, or at least eight genes chosen from ask, hom, metX, metY, metB, metC, metH, metE, metF, metK, ilvA, metQ, fprA, asd, cysD, cysN, cysC, pyc, cysH, cysI, cysY, cysX, cysZ, cysE, cysK, cysG, zwf hsk, mcbR and pepCK under conditions such that methionine is produced and recovering the methionine. In some embodiments, such a Cornyebacterium strain includes genetic alterations in at least eight genes.

In some embodiments, a method of culturing a recombinant microorganism described herein (e.g., a recombinant Cornyebacterium glutamicum) leads to production of methionine in an amount of at least 16 g per liter of culture.

In some embodiments, vectors include integration cassettes useful for integration of nucleic acid sequences into specific, desired genomic loci within the microorganism. In certain embodiments, integration cassettes modify an endogenous gene by inserting a heterologous nucleic acid sequence within the endogenous gene sequence. Such heterologous nucleic acid sequences may include, for example, nucleic acid sequences which express enzyme(s) in the methionine biosynthetic pathway. A heterologous gene can be a gene from a different organism, a modified endogenous gene, or an endogenous gene that has been moved from a different chromosomal location.

### BRIEF DESCRIPTION OF THE DRAWINGS

FIG. 1 is a schematic of the methionine biosynthetic pathway utilized in microorganisms described herein.

FIG. 2 is a schematic of the pH273 vector.

FIG. 3 is a schematic of the pH373 vector.

FIG. 4 is a schematic of the pH304 vector.

FIG. 5 is a schematic of the pH399 vector.

FIG. 6 is a schematic of the pH484 vector.

FIG. 7 is a schematic of the pH491 vector.

FIG. 8 is a schematic of the plasmid pOM62.

FIG. 9 is a schematic of the pH357 vector.

FIG. 10 is a schematic of the pH410 vector.

FIG. 11 is a schematic of the pH295 vector.

FIG. 12 is a schematic of the pH429 vector.

FIG. 13 is a schematic of the pH170 vector.

FIG. 14 is a schematic of the pH447 vector.

FIG. 15 is a schematic of the pH449 vector.

FIG. 16 is a schematic of the plasmid pOM423.

### DETAILED DESCRIPTION

The present invention is based, at least in part, on the discovery that certain genetic alterations in microorganisms lead to increased methionine production by the microorganisms. In another aspect, the present invention is based on the discovery that combinations of genetic alterations in certain genes are particularly favorable for methionine production.

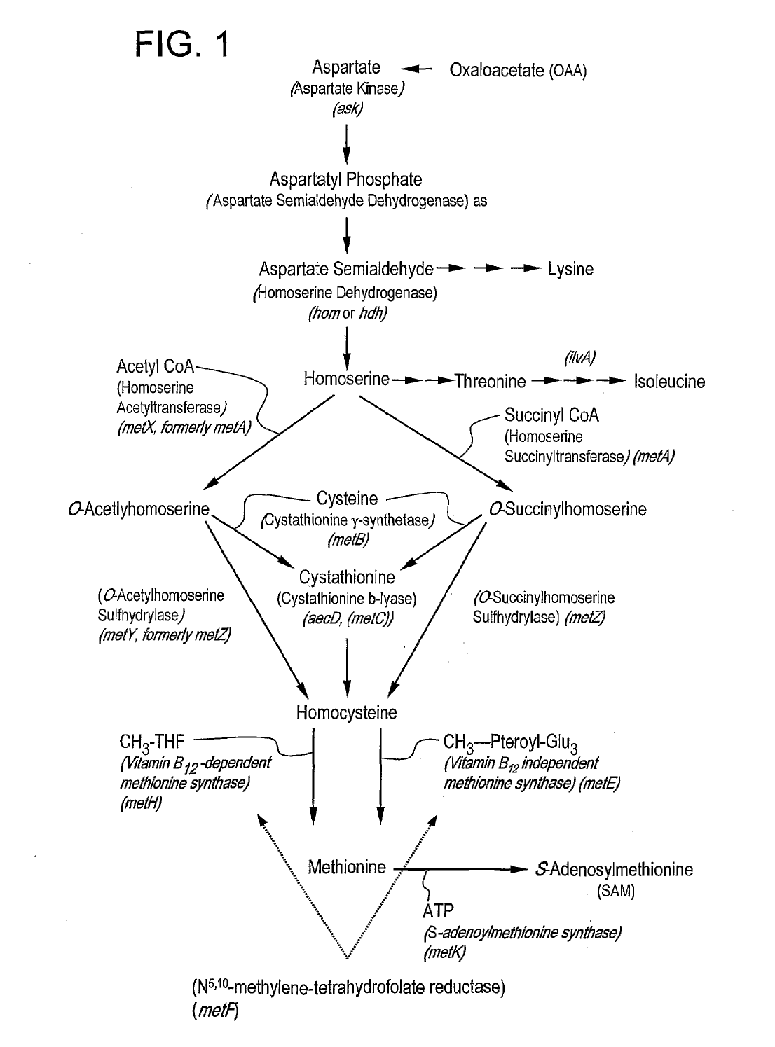

Two alternate pathways exist for the addition of sulfur atoms to intermediate substrates in methionine synthesis in microorganisms, as depicted in FIG. 1. For example, the bacterium Escherichia coli utilizes the transsulfuration pathway; whereas, some other microorganisms such as, for example, Saccharomyces cerevisiae and Corynebacterium glutamicum (C. glutamicum) employ a direct sulfhydrylation pathway. Although, many microorganisms appear to use one or the other pathway, C. glutamicum employs both pathways for methionine production.

This invention is based, at least in part, on the identification of genetic alterations which are beneficial for methionine production in Cornyebacterium, specifically, C. glutamicum. To maximize methionine production it is beneficial to decrease feedback inhibition of certain key enzymes in the pathway, such as, for example, Aspartate kinase (encoded by the ask gene), Homoserine dehydrogenase (encoded by the hom gene), O-Acetylhomoserine sulfhydrylase (encoded by the metY gene), Homoserine acetyltransferase (encoded by the metX gene), N5,10-Methylene tetrahydrofolate reductase (encoded by the metF gene) and Methionine synthases (encoded by genes metH and metE). For example, it has been reported that aspartate kinase enzymes (such as, for example, Ask), from various organisms, are inhibited by lysine and/or threonine. For example, changing amino acid at position 311 from threonine to isoleucine (T311L) reduces feedback inhibition of Ask in C. glutamicum (See U.S. Pat. No. 6,893,848, the entire disclosure of which is incorporated by reference herein). Similarly, homoserine dehydrogenase (Hom) can be inhibited by threonine, methionine, lysine and isoleucine, as described in: Sritharan V. Journal of General Microbiology, 136:203-209 (1990); Chassagnole C. et al. Biochemical Journal 356:415-23 (2001); Eikmanns B. J. et al. Antonie van Leeuwenhoek 64:145-63 (1993-94); and Cremer J. et al. Journal of General Microbiology 134(12):3221-3229 (1988)), the entire disclosures of which are incorporated by reference herein. Additionally, changing amino acid at position 393 from serine to phenylalanine (S393F) reduces feedback inhibition of Hom (also known as Hsdh) in C. glutamicum, as described in, Sugimoto M et al. Bioscience, Biotechnology & Biochemistry, 61:1760-1762 (1997), the entire disclosure of which is incorporated by reference herein. Additionally, the enzyme O-acetylhomoserine sulfhydrylase (MetY) is inhibited by methionine (WO 2004/108894 A2), as is methionine synthase (MetH) (Chen et al. J. Biol. Chem. 269:27193-27197 (1994)).

The instant invention demonstrates that it is beneficial to increase expression (e.g., transcription and/or translation) of certain genes in the methionine biosynthetic pathway, such as, for example, ask, hom (also known as hsd), metX (also known as metA), metY (also known as metZ), metB, metH, metE, metF, metC and/or certain genes of the cysteine biosynthetic pathway such as cysJ, cysE, cysK, cysN, cysD, cysH, cysA, cysI, cysG, cysZ, cysX, and cysM, in order to increase methionine production in microorganisms.

In addition, it is also beneficial to decrease or down regulate expression of certain genes whose products decrease methionine production under certain conditions, such as, for example, mcbR (also referred to as RXA00655), as described in Rey D. A., Journal of Biotechnology 103:51-65 (2003); and Rey D. A. et al., Molecular Microbiology 56:871-887 (2005), the entire disclosures of which are incorporated by reference herein, hsk, cysQ, cysY, ilvA, pepCK, metK, and metQ, in order to increase methionine production. For example, mutating the hsk gene which results in an enzyme with amino acid at position 190 changed from threonine to alanine (T190A), and/or mutating the metK gene to result in an S-Adenosylmethionine synthase enzyme with amino acid at position 94 changed from cysteine to alanine (C94A), is particularly beneficial for increasing methionine production in C. glutamicum.

This invention further features microorganisms which contain genetic alterations in each gene in a combination of any two, or a combination of any three, or a combination of any four, or a combination of any five, or a combination of any six; or a combination of any seven; or a combination of any eight of the following genes: ask^{fbr}, hom^{fbr}, metX (also referred to as metA), metY (also referred to as metZ), metB, metH, metE, metF and zwf, where the genetic alterations lead to overexpression of the any two, or any three, or any four, or any five, or the any six, or the any seven, or the any eight genes, thereby resulting in increased production of methionine relative to methionine produced in the absence of the genetic alterations. Also featured by the instant invention are microorganisms that contain genetic alterations in each of the nine genes listed above, which enhance the expression of all nine of the above recited genes, thereby increasing methionine production.

In some embodiments, recombinant microorganisms described herein contain genetic alterations in each of any two, or any three, or any four, or any five, or six, or seven, or eight, or nine of the following genes: ask^{fbr}, hom^{fbr}, metX, metY, metB, metH, metE, metF and zwf, in combination with genetic alterations in at least one of the following genes: mcbR, hsk, metQ, metK and pepCK, thereby to increase methionine production. It is understood that enhancing or increasing expression encompasses increasing transcription/translation of a gene or increasing activity or level of a protein/enzyme encoded by the gene. Similarly, decreasing expression encompasses decreasing transcription/translation of a gene or decreasing activity/level of a protein/enzyme encoded by the gene.

In order that the present invention may be more readily understood, certain terms are first defined herein.

The phrase a “methionine-producing microorganism,” as used herein, refers to any microorganism capable of producing methionine, e.g., bacteria, yeasts, fungi, Archaea etc. In some embodiments, a methionine producing microorganism belongs to the genus Corynebacterium. In yet other embodiments, a methionine producing microorganism is Corynebacterium glutamicum. In yet other embodiments, a methionine producing microorganism is chosen from: a microorganism belonging to the genus Corynebacterium, a microorganism belonging to the genus Enterobacteria, a microorganism belonging to the genus Bacillus, and a yeast. In some embodiments, a microorganism belonging to the genus Corynebacterium is Corynebacterium glutamicum; a microorganism belonging to the genus Enterobacteria is Escherichia coli. In other embodiments a microorganism belonging to the genus Bacillus is Bacillus subtlis. In yet other embodiments, a yeast is Saccharomyces cerevisiae.

As used herein, the phrase “increased levels of methionine production” refers to a titer of methionine (for example, in g/l under suitable fermentation conditions) produced by a microorganism including genetic alterations in two or more, or three or more, or four or more, or five or more, or six or more, or seven or more, or eight or more, or nine or more, or ten or more, or eleven or more, or twelve or more, or thirteen or more, or fourteen or more, or fifteen or more, or sixteen or more, or seventeen or more, or eighteen or more, or nineteen or more, or twenty or more, or twenty one or more, or twenty two or more, or twenty three or more, or twenty four or more, or twenty five or more, or twenty six or more, or twenty seven or more, or twenty eight or more, or twenty nine or more, or thirty or more, or thirty one or more, or thirty two or more, or thirty three or more, or thirty four or more genes, as described herein, where such titer is greater than the amount produced under similar fermentation conditions by a control microorganism, which is usually the microorganism lacking such genetic alterations. The phrase “increased levels of methionine” also refers to titer of methionine produced by recombinant microorganisms including at least two deregulated proteins described herein. The phrase “increased levels of methionine production” includes values and ranges of methionine included and/or intermediate of the values set forth herein. Increased levels of methionine production are also intended to encompass titers produced above a basal level established by microorganisms that have not been genetically engineered to express a heterologous methionine insensitive biosynthetic enzyme. In some embodiments, increased levels of methionine refer to a titer of methionine produced by a genetically engineered (e.g., modified or altered) microorganism relative to the amount produced by its wild-type or parental counterpart or by the strain that immediately preceded the genetically engineered strain during the strain construction, as discussed in the Examples herein.

The terms “biosynthetic pathway” and “biosynthetic process” as used herein refer to an in vivo or in vitro process by which a molecule or compound of interest is produced as the result of one or more biochemical reactions. Generally, beginning with a precursor molecule, a prototypical biosynthetic process involves the action of one or more enzymes functioning in a stepwise fashion to produce a molecule or compound of interest. Molecules or compounds of interest include, for example, small organic molecules, amino acids, peptides, cellular cofactors, vitamins and similar chemical entities. Molecules or compounds of interest particularly include chemicals such as methionine, homocysteine, S-adenosylmethionine, glutathione, cysteine, biotin, thiamine, mycothiol, coenzyme A, coenzyme M, and lipoic acid. In certain circumstances, an enzyme or enzymes functioning in a biosynthetic pathway may be regulated by chemical products generated in the process. In such cases, a feedback loop is said to exist such that increasing concentrations of an end or intermediate product modify the functioning or activity of enzymes within the pathway. For example, the ultimate product or an intermediate of a biosynthetic pathway may act to down-regulate the level or activity of an enzyme in the biosynthetic process, thereby decreasing the rate at which a desired end product is produced. Situations such as this are often undesirable, for example, in large scale fermentative processes used in industry for the production of molecules or compounds of interest. The methods and materials discussed herein are directed, at least in part, to increasing industrial scale and fermentative production of compounds of interest. A typical example of a feedback loop occurs in the production of methionine described herein.

The term “methionine biosynthetic pathway” refers to a biosynthetic pathway involving methionine biosynthetic enzymes (e.g. polypeptides encoded by biosynthetic enzyme-encoding genes), compounds (e.g., precursors, substrates, intermediates or products), cofactors and the like utilized in the formation or synthesis of methionine. The term “methionine biosynthetic pathway” includes biosynthetic pathway(s) leading to the synthesis of methionine in a microorganism (e.g., in vivo) as well as biosynthetic pathway(s) leading to the synthesis of methionine in vitro. FIG. 1 depicts a schematic representation of the methionine biosynthetic pathway.

The term “methionine biosynthetic enzyme,” as used herein, refers to any enzyme utilized in the formation of a compound (e.g., intermediate or product) of the methionine biosynthetic pathway. “Methionine biosynthetic enzyme” includes enzymes involved in e.g., the “transsulfulration pathway” and in the “direct sulfhydrylation pathway,” alternate pathways for the synthesis of methionine. For example, as discussed above, E. coli utilizes a transsulfuration pathway, whereas, other microorganisms such as Saccharomyces cerevisiae, C glutamicum, and B. subtilis and relatives of these microorganisms employ a direct sulfhydrylation pathway. Although, many microorganisms use either the transsulfuration pathway or the direct sulfhydrylation pathway, but not both, some microorganisms, such as for example, C. glutamicum, use both pathways for the synthesis of methionine.

As depicted in FIG. 1, synthesis of methionine from oxaloacetate (OAA) proceeds via the intermediates, aspartate, aspartate (aspartyl) phosphate and aspartate semialdehyde. Aspartate semialdehyde is converted to homoserine by homoserine dehydrogenase (the product of the hom gene, also known as thrA, metL, hdh, hsd, among other names in other organisms). The subsequent steps in methionine synthesis can proceed through the transsulfuration pathway and/or the direct sulfhydrylation pathway.

In the transsulfuration pathway, homoserine is converted to either O-acetylhomoserine by homoserine acetyltransferase (the product of the metX gene, also referred to as metA) and the additional substrate acetyl CoA, or to O-succinylhomoserine by use of the additional substrate succinyl CoA and the product of the meta gene (Homosenine succinyltransferase). Donation of a sulfur group from cysteine to either O-acetylhomoserine or O-succinylhomoserine by Cystathionine γ-synthase, the product of the metB gene, produces cystathionine. Cystathionine is then converted to homocysteine by Cystathionine β-lyase, the product of the metC gene (also referred to as the aecD gene in some microorganisms).

In the direct sulfhydrylation pathway, O-acetylhomoserine sulfhydrylase, the product of the metY gene (also referred to as the metZ gene) catalyzes the direct addition of sulfide to O-acetylhomoserine to form homocysteine. Homocysteine can also be formed in a variation of the direct sulfhydrylation pathway by the direct addition of a sulfide group to O-succinylhomoserine by O-Succinylhomoserine sulfhydralase, the product of the metZ gene. As used herein, metY is used interchangeably with metZ, and metA is used interchangeably with metX.

Unlike the transsulfuration/sulfhydrylation enzymes that are present only in organisms with de novo methionine synthesis, methionine synthase is present in many additional organisms to ensure regeneration of the methyl group of S-adenosylmethionine (SAM). Two types of methionine synthases can perform this function in E. coli, vitamin B₁₂-dependent methionine synthase (the product of the metH gene) and vitamin B₁₂-independent methionine synthase (the product of the metE gene). The methyl group of methionine is donated by methyl-tetrahydrofolate (methyl-THF), either with or without a polyglutamate tail, which is formed by reduction of methylene-THF in a reaction catalyzed by the metF gene product. S-adenosylmethionine synthase, encoded by the metK gene, is responsible for the formation of SAM from methionine and ATP.

Additionally, cysteine can be used as a sulphur donor in methionine biosynthesis in the trans-sulfuration pathway. In bacteria, cysteine is synthesized from serine by incorporation of sulfide or a sulfur atom from thiosulfate. The gene product of the cysK gene (O-acetylserine (thiol)-lyase A or CysK) synthesizes cysteine from O-acetylserine and sulfide, while the gene product of the cysM gene (O-acetylserine (thiol)-lyase B or Cys M) utilizes thio-sulfate instead of sulfide in the synthesis of cysteine.

When the ultimate source of sulfur is sulfate, a series of enzymes are required to reduce the sulfate to sulfide for cysteine and methionine biosynthesis. Usually, sulfate is taken up by cells with the help of transport proteins encoded by genes such as cysZ (sulfate transporter) or cysP. Sulfate is activated by products of the cysD (sulfate adenylyltransferase subunit 2) and cysN (sulfate adenyltransferase subunit 1) genes to generate adenosyl-phospho-sulfate (also referred to as APS). It has been reported that in some organisms, adenosyl-phospho-sulfate is then activated in a further step by a protein with adenosyl-phospho-sulfate-kinase activity to yield phosphoadenosyl-phospho-sulfate (referred to as PAPS), which is subsequently reduced by the enzyme, PAPS-reductase, encoded by the cysH gene. Alternatively, APS can be directly reduced to yield sulfite by an APS-reductase enzyme.

Since no gene encoding for a protein with the activity of an adenosyl-phospho sulfate kinase activity has yet been identified in C glutamicum, it remains unclear whether adenosyl-phospho sulfate or phosphoadenylyl-phospho-sulfate is the substrate for the enzyme encoded by the cysH gene. The product of the reduction step is sulfite, which is further reduced by the activity of the sulfite reductase enzyme encoded for by the genes cysI (sulfite reductase subunit 1) and cysJ (sulfite reductase subunit 2).

The precursor for cysteine biosynthesis is usually derived from serine, which is converted to O-acetyl serine by the activity of serine-acetyltransferase (encoded by the gene cysE). O-acetyl-serine and sulfide act as substrates for the enzyme O-acetylserine (thiol) lyase A, encoded by the cysK gene. In the case of thiosulfate as a sulphur source, a second cysteine synthase has been described in certain organisms including E. Coli and S typhimurium (See, for example, Neidhardt F C ed. ASM Press Washington (1996)) that use O-acetyl-serine and thiosulfate to generate sulfocysteine. The gene coding for the second cysteine synthase enzyme is referred to as cysM (O-acetylserine (thiol) lyase A) which is also found in C. glutamicum.

Table 1a lists various enzymes in the methionine biosynthetic pathway and the corresponding genes encoding them. Table 1b lists various enzymes in the cysteine biosynthetic pathway and the corresponding genes encoding them. Table 1c lists additional proteins and enzymes that affect methionine biosynthesis directly or indirectly, and the corresponding genes. For the purpose of convenience, genes featured herein are each assigned a letter code. It is understood that in some microorganisms the names of the genes encoding the corresponding enzymes may vary from the names listed herein.

TABLE 1a
Enzymes in the methionine biosynthetic
pathway and the genes encoding them

| Enzyme | Gene | Letter Code | |
|---|---|---|---|
| Aspartate kinase | ask | A | (+) |
| Homoserine Dehydrogenase | hom | D | (+) |
| Homoserine Acetyltransferase | metX | X | (+) |
| Homoserine Succinyltransferase | metA | S | (+) |
| (for example, in E. coli) | | | |
| Cystathionine γ-synthetase | metB | B | (+) |
| Cystathionine β-lyase | metC | C | (+) |
| O-Acetylhomoserine sulfhydrylase | metY | Y | (+) |
| O-Succinylhomoserine sulfhydrylase | metZ | Z | (+) |
| (for example, in Rhizobium) | | | |
| Vitamin B12-dependent methionine synthase | metH | H | (+) |
| Vitamin B12-independent methionine synthase | metE | E | (+) |
| N5,10-methylene-tetrahydrofolate reductase | metF | F | (+) |
| S-adenosylmethionine synthase | metK | K | (−) |
| D-methionine binding lipoprotein or subunit | metQ | Q | (−) |
| of methionine uptake system | | | |

(+): Refers to genes overexpression of which is desirable for increased production of methionine
(−): Refers to genes lowering or decreasing the expression or activity of which is desirable for increased production of methionine

TABLE 1b
Enzymes in the cysteine biosynthetic
pathway and genes encoding them

| Enzyme | Gene | Letter Code | |
|---|---|---|---|
| Sulfate adenylyltransferase subunit 2 | cysD | cD | (+) |
| Gamma-cystathionase | cysA | cA | (+) |
| Sulfate adenylyltransferase subunit 1 | cysN | cN | (+) |
| APS kinase (for example, in E. coli) | cysC | Cc | (+) |
| APS reductase (for example, in | cysH | cH | (+) |
| C. glutamicum.), PAPS reductase | | | |
| (for example, in E. coli) | | | |
| Sulfite reductase subunit 1 | cysI | cI | (+) |
| Sulfite reductase subunit 2 (in E. coli) | cysJ | cJ | (+) |
| Cystathionine beta synthase(reverse pathway) | cysY | cY | (−) |
| Accessory role sulfite reduction | cysX | cX | (+) |
| Sulfate transporter | cysZ | cZ | (+) |
| Serine O-acetyltransferase | cysE | cE | (+) |
| O-acetylserine (thiol)-lyase A | cysK | cK | (+) |
| O-acetylserine (thiol)-lyase A (for example, | cysM | cM | (+) |
| E. coli, etc) | | | |
| Uroporphyrinogen III synthase | cysG | cG | (+) |
| APS phosphatase (for example, in E. coli) | cysQ | cQ | (−) |

(+): Refers to genes overexpression of which is desirable for increased production of methionine
(−): Refers to genes lowering or decreasing the expression or activity of which is desirable for increased production of methionine

TABLE 1c
Additional genes that may be altered to increase methionine production

| Enzyme/Protein | Gene | Letter Code | |
|---|---|---|---|
| Glucose-6-phosphate dehydrogenase | zwf | W | (+) |
| Homoserine kinase | hsk | V | (−) |
| TetR-type transcriptional regulator of | mcbR | R | (−) |
| sulfur metabolism | | | |
| Phosphoenolpyruvate carboxykinase | pepCK | P | (−) |
| Pyruvate carboxylase | pyc | Py | (+) |
| NADP-ferredoxin reductase | fprA | Fp | (+) |
| Aspartate semialdehyde dehydrogenase | asd | As | (+) |
| Threonine dehydratase, biosynthetic | ilvA | Iv | (−) |
| Threonine dehydratase, catabolic | Cgl 0978, | T | (−) |
| | tdh | | |

(+): Refers to genes overexpression of which is desirable for increased production of methionine
(−): Refers to genes lowering or decreasing the expression of which is desirable for increased production of methionine

Exemplary combinations of genes that may be altered to increase methionine production are depicted in Table II. However, it is understood that any combination of genes may be altered, so long as the combination results in enhanced methionine production.

TABLE II
Exemplary combinations of altered genes

A, D, X, Y, B
A, D, X, Y, H
A, D, X, Y, E
A, D, X, Y, F
A, D, X, Y, W
A, D, X, B, H
A, D, X, B, E
A, D, X, B, F
A, D, X, B, W
A, D, X, H, E
A, D, X, H, F
A, D, X, H, W
A, D, X, E, W
A, X, Y, B, H
A, X, Y, B, E
A, X, Y, B, F
A, X, Y, B, W
A, X, Y, H, E
A, X, Y, H, F
A, X, Y, H, W
A, X, Y, E, F,
A, X, Y, E, W
A, D, X, E, F

Recombinant microorganisms encompassed by this invention may be genetically engineered to include alteration of endogenous genes which leads to an increase in methionine production, for example, by introducing alterations in genes that either increase the expression or decrease the expression of certain genes. Alternatively, recombinant microorganisms maybe genetically manipulated to express enzymes/proteins encoded by heterologous genes that are introduced into such microorganisms. In some embodiments, recombinant microorganisms are genetically engineered to alter expression of a combination of certain enzymes/proteins, where such combination leads to increased methionine production relative to methionine production in the absence of the combination. Expression of a combination of suitable enzymes/proteins can be achieved, for example, by altering the expression of endogenous genes and/or introducing heterologous genes into the microorganism.

Table III below includes Genbank Accession numbers for various genes isolated from C. glutamicum and proteins encoded by them, where various combinations of genes can be altered, thereby leading to enhanced methionine production.

TABLE III
Genbank Accession numbers for various C. glutamicum genes involved
in methionine biosynthesis and proteins encoded by them

| | Gene | Protein |
|---|---|---|
| Gene name | accession | accession |
| MetK | Cgl1603 | BAB98996.1 |
| Hom | Cgl1183 | BAB98576.1 |
| hsk/thrA | Cgl1184 | BAB98577.1 |
| metY/Z | Cgl0653 | BAB98046.1 |
| metA/X | Cgl0652 | BAB98045.1 |
| MetH | Cgl1507 | BAB98900.1 |
| MetE | Cgl1139 | BAB98532.1 |
| MetF | Cgl2171 | BAB99564.1 |
| MetC | Cgl2309 | BAB99702.1 |
| MetB | Cgl2446 | BAB99839.1 |
| ask/lysC | Cgl0251 | BAB97644.1 |
| Asd | Cgl0252 | BAB97645.1 |
| Zwf | Cgl1576 | BAB98969.1 |
| PepCK | Cgl1585 | BAB98978.1 |
| CysE | Cgl2563 | BAB99956.1 |
| cysH (encodes PAPS or APS reductase) | Cgl2816 | BAC00210.1 |
| gene encoding sulfite reductase | Cgl2817 | BAC00211.1 |
| cysJ/fprA | Cgl2818 | BAC00212.1 |
| cysN encoding sulfate adenylate transferase | Cgl2814 | BAC00208.1 |
| subunit 1 | | |
| cysD encoding sulfate adenylate transferase | Cgl2815 | BAC00209.1 |
| subunit 2 | | |
| gene encoding sulfate permease | Cgl1473 | BAB98866.1 |
| gene encoding sulfate permease | Cgl1051 | BAB98444.1 |
| gene encoding sulfate transport system | Cgl0870 | BAB98263.1 |
| permease protein | | |
| gene encoding sulfate permease | Cgl2812 | BAC00206.1 |
| gene encoding sulfate permease | Cgl2813 | BAC00207.1 |
| CysG | Cgl1998 | BAB99391.1 |
| CysK | Cgl2562 | BAB99955.1 |
| CysM | Cgl2136 | BAB99529.1 |
| gene encoding pyruvate carboxylase | Cgl0689 | BAB98082.1 |

In some embodiments, methionine producing microorganisms encompassed by the present invention contain genetic alterations in each of any two genes, or any three genes, or any four genes, or any five genes chosen from: ask^{fbr}; hom^{fbr}; metX; metY; metB; metH; metE; metF; and zwf. This invention further features microorganisms containing genetic alterations that include genetic alterations in each of any six genes chosen from ask^{fbr}, hom^{fbr}, metX, metY, metB, metH, metE, metF and zwf. Additionally, the present invention features microorganisms containing genetic alterations in each of any seven genes, or each of any eight genes, or nine genes chosen from ask^{fbr}, hom^{fbr}, metX, metY, metB, metH, metE, metF and zwf.

The number of possible combinations of the various genes that may be altered can be calculated, for example, based on the following equation:

$$\frac{n !}{( n - r ) ! Xr !}$$

where n is the total number of genes that may be altered and r is the number of genes that are altered in a microorganism. Accordingly, the number of possible combinations of any two genes chosen from ask^{fbr}, hom^{fbr}, metX, metY, metB, meth, metE, metF and zwf, that may be altered, can be calculated as follows:

$$\frac{9 !}{( 9 - 2 ) ! X 2 !} = 36.$$

Similarly, the number of possible combinations of any five genes chosen from ask^{fbr}, hom^{fbr}, metX, metY, metB, metH, metE, metF and zwf that may be altered, can be calculated as follows:

$$\frac{9 !}{( 9 - 5 ) ! X 5 !} = 126.$$

Therefore, based on the above formula, the number of possible combinations of any five genes, or any six genes, or any seven genes, or any eight genes, or nine genes chosen from ask^{fbr}, hom^{fbr}, metX, metY, metB, metH, metE, metF and zwf, that may be altered is 126, 84, 36, 9 and 1 respectively.

Similarly, number of possible combinations of any of the altered genes, as described herein, can be easily determined based on the above formula The phrase “insensitive to methionine feedback,” as used herein, refers to an enzyme that is capable of enzymatically functioning at a significant level in the presence of methionine and has a specific activity that is at least 20% of the activity in the absence of methionine. An enzyme that is insensitive to methionine feedback may function well in the presence of; for example, 1-10 μM, 10-100 μM or 100 μM-1 mM methionine. In some embodiments, an enzyme of interest is capable of functioning at concentrations of 1-10 mM, 10-100 mM methionine or at even higher concentrations. Also, in their native state, some methionine biosynthetic enzymes are sensitive to feedback inhibition by other amino acids, such as threonine and lysine. This invention features, at least in part, methionine, lysine, and/or threonine feedback insensitive enzymes which are involved in methionine biosynthetic pathways or processes which result in the production of methionine, such as, for example, Ask^{fbr} and Hom^{fbr}.

In some embodiments, a microorganism featured herein belongs to the genus Corynebacterium. In other embodiments, a microorganism is Corynebacterium glutamicum. In yet other embodiments, a microorganism is chosen from: Gram-negative bacteria (e.g., Escherichia coli or related Enterobacteria), Gram-positive bacteria (e.g., Bacillus subtilis or related Bacillus), yeast (e.g., Saccharomyces cerevisiae or related yeast strains), and Archaea.

In some embodiments, a microorganism described herein has deregulation of at least two, or at least three, or at least four, or at least five methionine biosynthetic enzymes. In other embodiments, a microorganism described herein has deregulation of at least six methionine biosynthetic enzymes. In some embodiments, a microorganism described herein has deregulation of at least seven or more methionine biosynthetic enzymes. The term “deregulation,” as used herein, refers to either an increase in level and/or activity or a decrease in level and/or activity or complete absence, of a biosynthetic enzyme relative to the level and/or specific activity of its parental or wild-type counterpart. In some embodiments, a “deregulated” biosynthetic enzyme is encoded by a gene that is altered, as described herein. For example, a “deregulated” biosynthetic enzyme may either be produced, for example, by altering an endogenous gene encoding, the enzyme, or by introducing a heterologous gene into a microorganism which produces the enzyme.

In other embodiments, a microorganism described herein has two or more, or three or more, or four or more, or five or more, or six or more enzymes from the cysteine biosynthetic pathway that are deregulated. In yet other embodiments, microorganisms described herein have two or more enzymes from the methionine biosynthetic pathway and two or more enzymes from the cysteine biosynthetic pathway that are deregulated. For example, in some embodiments, recombinant microorganisms include five or more enzymes from the methionine biosynthetic pathway and six or more enzymes from the cysteine biosynthetic pathway that are deregulated. Further, enzymes/proteins that directly or indirectly affect genes in methionine biosynthetic pathway and/or cysteine biosynthetic pathway can also be deregulated, for example, reduced in level and/or activity, thereby to increase methionine production. For example, in some embodiments, recombinant microorganisms include genetic alterations in at least two genes, where such alterations result in deregulation of at least two proteins chosen from: APS phosphatase; Cystationine beta synthase(reverse pathway), homoserine kinase; TetR-type transcriptional regulator of sulfur metabolism; D-methionine binding lipoprotein, phosphoenolpyruvate carboxykinase, S-adenosylmethionine synthase, and threonine dehydratase, encoded by the genes.

In some embodiments, the present invention features new and improved methods of producing methionine using genetically altered microorganisms in which the methionine biosynthetic pathway has been manipulated such that the microorganisms have the ability to produce methionine at an increased level relative to methionine produced in absence of the genetic alterations.

The new and improved methodologies described herein include methods of producing methionine in microorganisms including at least two, or at least three, or at least four, or at least five, or at least six, or at least seven, or at least eight or more enzymes of the methionine biosynthetic pathway that are deregulated, such that methionine is produced at an increased level relative to the microorganism without such a deregulation. For example, in some embodiments, microorganisms described herein include genetic alterations in five or more genes, which result in deregulation of the five or more enzymes encoded by the genes, where the enzymes are chosen from: aspartate kinase, homoserine dehydrogenase, homoserine acetyltransferase, cystathionine γ-synthetase, O-acetylhomoserine sulfhydralase, O-succinylhomoserine sulfydralase, Vitamin-B12-dependent methionine synthase, N5,10-methylene-tetrahydrofolate reductase, S-adenosylmethionine synthase, cystathionine-β-lyase, homoserine succinyltransferase, and Vitamin-B12-independent methionine synthase.

The methodologies of increasing methionine production described herein also include methods of producing microorganisms with genetic alteration(s) in genes in the cysteine biosynthetic pathway, such that methionine is produced at increased level relative to the level in absence of the genetic alterations.

For example, in some embodiments, microorganisms described herein include genetic alterations in two or more, or three or more, or four or more, or five or more, or six or more, or seven or more genes, which result in deregulation of the enzymes encoded by the genes, where the enzymes are chosen from: sulfite adenylyltransferase subunit 2, sulfate adenylyltransferase subunit 1, cystathionine beta synthetase, APS kinase, APS reductase, PAPS reductase, sulfite reductase subunit 1, sulfite reductase subunit 2, accessory role sulfite reduction, sulfate transporter, serine O-acetyltransferase, O-acetylserine (thiol)-lyase A, uroporphyrinogen III synthase, APS phosphatase and gamma cystathionase. In some embodiments, recombinant microorganisms include six deregulated enzymes of the cysteine biosynthetic pathway.

The methodologies described herein feature microorganisms, e.g., recombinant microorganisms, as well as vectors and genes (e.g., wild-type and/or mutated genes) as described herein and/or cultured in a manner which results in the increased production of methionine.

The term “recombinant microorganism” refers to a microorganism (e.g., bacteria, yeast cell, fungal cell, etc.) that has been genetically altered, modified or engineered (e.g., genetically engineered) using, for example, in vitro DNA manipulation techniques or classical in vivo genetic techniques, such that it exhibits an altered, modified or different genotype and/or phenotype (e.g., when the genetic modification affects coding nucleic acid sequences of the microorganism) as compared to the naturally-occurring microorganism from which it was derived.

A “recombinant microorganism” described herein may be genetically engineered to include genetic alterations in at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least twenty one, or at least twenty two, or at least twenty three, or at least twenty four, or at least twenty five genes, or all twenty six genes chosen from ask, hom, metX, metB, metC, metY, metH, mete, metF, cysE, cysK, cysM, cysD, cysA, cysN, cysH, cysI, cysJ, cysX, cysZ, cysC, cysG, zwf, pyc, fprA and asd, where the genetic alterations lead to overexpression of the genes. In some embodiments, a “recombinant microorganism” described herein may be genetically engineered to include genetic alterations in at least two genes, or at least three genes, or at least four genes, or at least five genes, or at least six genes, or at least seven genes or at least eight genes chosen from metK, metQ, cysY, cysQ, hsk, mcbR, pepCK and ilvA, where the genetic alterations lead to decreasing the expression of the genes. In other embodiments, embodiments, “recombinant microorganisms” include genetic alterations in some genes, which increase the expression of those genes, and genetic alterations in other genes, which decrease the expression of such genes, thereby resulting in increased methionine production by the recombinant microorganism.

The skilled artisan will appreciate that a microorganism expressing a gene at increased level produces the resultant gene product at an increased level and/or activity relative to a microorganism in absence of increased expression of gene. Similarly, a microorganism including decreased expression of a gene produces the resultant gene product at a lower level and/or activity relative to a microorganism in absence of decreased expression of the gene.

The term “recombinant microorganism,” as used herein, also refers to a microorganism that has been engineered (e.g., genetically engineered) or modified such that the microorganism has at least two enzymes of the methionine biosynthetic pathway and/or at least two enzymes of the cysteine biosynthetic pathway deregulated such that methionine is produced at increased levels. In some embodiments, recombinant microorganisms include at least five enzymes of the methionine biosynthetic pathway and at least six enzymes of the cysteine biosynthetic pathway that are deregulated such that methionine is produced at increased levels. Modification or engineering of such microorganisms can be achieved according to any methodology described herein or known in the art, including, but not limited to, alteration of a gene encoding a biosynthetic pathway enzyme.

The terms “deregulated” or “manipulated,” as used in reference to an enzyme or protein, are used interchangeably herein, and refer to an enzyme or protein, the activity or level of which has been altered or modified such that the level or rate of flux through at least one upstream or downstream precursor or intermediate, substrate or product of the enzyme is altered or modified, for example, as compared to a corresponding wild-type or naturally occurring enzyme or protein. A “manipulated” enzyme (e.g., a “manipulated” biosynthetic enzyme) includes an enzyme, the expression, production, or activity of which has been altered or modified such that at least one upstream or downstream precursor, substrate or product of the enzyme is altered or modified (e.g., an altered or modified level, ratio, etc. of precursor, substrate and/or product), for example, as compared to a corresponding wild-type or naturally occurring enzyme. A “manipulated” enzyme also includes one where resistance to inhibition, e.g., feedback inhibition, by one or more products or intermediates has been enhanced. For example, an enzyme that is capable of enzymatically functioning efficiently in the presence of, e.g., methionine.

The terms “overexpress,” “overexpressing,” “overexpressed” and “overexpression” refer to expression of a gene product (e.g., a methionine biosynthetic enzyme or sulfate reduction pathway enzyme or cysteine biosynthetic enzyme) at a level greater than that present prior to a genetic alteration of the microorganism or in a comparable microorganism which has not been genetically altered. In some embodiments, a microorganism can be genetically altered (e.g., genetically engineered) to express a gene product at an increased level relative to that produced by an unaltered microorganism or in a comparable microorganism which has not been altered. Genetic alteration includes, but is not limited to, altering or modifying regulatory sequences or sites associated with expression of a particular gene (e.g., by adding strong promoters, inducible promoters or multiple promoters or by removing regulatory sequences such that expression is constitutive), modifying the chromosomal location of a particular gene, altering nucleic acid sequences adjacent to a particular gene such as a ribosome binding site or transcription terminator, increasing the copy number of a particular gene, modifying proteins (e.g., regulatory proteins, suppressors, enhancers, transcriptional activators and the like) involved in transcription of a particular gene and/or translation of a particular gene product, or any other conventional means of deregulating expression of a particular gene routine in the art (including but not limited to use of antisense nucleic acid molecules, for example, to block expression of repressor proteins) and/or the use of mutator alleles, e.g., bacterial alleles that enhance genetic variability and accelerate, for example, adaptive evolution).

In some embodiments, a microorganism can be physically or environmentally altered to express a gene product at an increased or lower level relative to level of expression of the gene product by an unaltered microorganism or comparable microorganism which has not been altered. For example, a microorganism can be treated with or cultured in the presence of an agent known or suspected to increase transcription of a particular gene and/or translation of a particular gene product such that transcription and/or translation are enhanced or increased. Alternatively, a microorganism can be cultured at a temperature selected to increase transcription of a particular gene and/or translation of a particular gene product such that transcription and/or translation are enhanced or increased.

The terms “deregulate,” “deregulated” and “deregulation” refer to alteration or modification of at least one gene in a microorganism, wherein the alteration or modification results in increasing methionine production in the microorganism relative to methionine production in absence of the alteration or modification. In some embodiments, a gene that is altered or modified encodes an enzyme in a biosynthetic pathway, such that the level or activity of the biosynthetic enzyme in the microorganism is altered or modified. In some embodiments, at least one gene that encodes an enzyme in a biosynthetic pathway is altered or modified such that the level or activity of the enzyme is enhanced or increased relative to the level in presence of the unaltered or wild-type gene. In other embodiments, at least two, or at least three, or at least four, or at least five genes that encodes an enzyme in a biosynthetic pathway are altered or modified such that the level or activity of the enzymes encoded by the genes is decreased or lowered relative to the level in presence of the unaltered or wild-type gene. In some embodiments, the biosynthetic pathway is the methionine biosynthetic pathway. In other embodiments, the biosynthetic pathway is the cysteine biosynthetic pathway. Deregulation also includes altering the coding region of one or more genes to yield, for example, an enzyme that is feedback resistant or has a higher or lower specific activity. Also, deregulation further encompasses genetic alteration of genes encoding transcriptional factors (e.g., activators, repressors) which regulate expression of genes in the methionine and/or cysteine biosynthetic pathway.

The phrase “deregulated pathway” refers to a biosynthetic pathway in which at least one gene that encodes an enzyme in a biosynthetic pathway is altered or modified such that the level or activity of at least one biosynthetic enzyme is altered or modified. The phrase “deregulated pathway” includes a biosynthetic pathway in which more than one gene has been altered or modified, thereby altering level and/or activity of the corresponding gene products/enzymes. In some cases the ability to “deregulate” a pathway (e.g., to simultaneously deregulate more than one gene in a given biosynthetic pathway) in a microorganism arises from the particular phenomenon in microorganisms in which more than one enzyme (e.g., two or three biosynthetic enzymes) are encoded by genes occurring adjacent to one another on a contiguous piece of genetic material termed an “operon.” In other cases, in order to deregulate a pathway, a number of genes are deregulated in a series of sequential engineering steps.

The term “operon” refers to a coordinated unit of genetic material that contains a promoter and possibly a regulatory element associated with one or more, preferably at least two, structural genes (e.g., genes encoding enzymes, for example, biosynthetic enzymes). Expression of the structural genes can be coordinately regulated, for example, by regulatory proteins binding to the regulatory element or by anti-termination of transcription. The structural genes can be transcribed to give a single mRNA that encodes all of the structural proteins. The term “operon” includes at least two adjacent genes or ORFs, optionally overlapping in sequence at either the 5′ or 3′ end of at least one gene or ORF. The term “operon” includes a coordinated unit of gene expression that contains a promoter and possibly a regulatory element associated with one or more adjacent genes or ORFs (e.g., structural genes encoding enzymes, for example, biosynthetic enzymes). Expression of the genes can be coordinately regulated, for example, by regulatory proteins binding to the regulatory element or by anti-termination of transcription. The genes of an operon (e.g., structural genes) can be transcribed to give a single mRNA that encodes all of the proteins. Due to the coordinated regulation of genes included in an operon, alteration or modification of the single promoter and/or regulatory element can result in alteration or modification of each gene product encoded by the operon. Alteration or modification of a regulatory element includes, but is not limited to, removing endogenous promoter and/or regulatory element(s), adding strong promoters, inducible promoters or multiple promoters or removing regulatory sequences such that expression of gene products is modified, modifying the chromosomal location of the operon, altering nucleic acid sequences adjacent to the operon or within the operon such as a ribosome binding site, codon usage, increasing copy number of the operon, modifying proteins (e.g., regulatory proteins, suppressors, enhancers, transcriptional activators and the like) involved in transcription of the operon and/or translation of the gene products of the operon, or any other conventional means of deregulating expression of genes routine in the art (including, but not limited to, use of antisense nucleic acid molecules, for example, to block expression of repressor proteins).

In some embodiments, recombinant microorganisms described herein have been genetically engineered to overexpress a bacterially-derived gene or gene product. The terms “bacterially-derived” and “derived-from bacteria” refer to a gene which is naturally found in bacteria or a gene product which is encoded by a bacterial gene.

In some embodiments, recombinant microorganisms described herein include genetic alterations in each gene in a combination of any two genes, or a combination of any three genes, or a combination of any four genes, or a combination of any five genes, or a combination of any six genes, or a combination of any seven genes, or a combination of any eight genes, or a combination of any nine genes, or a combination of any ten genes, or a combination of any eleven genes, or a combination of any twelve genes, or a combination of any thirteen genes, or a combination of any fourteen genes, or a combination of any fifteen genes, or a combination of any sixteen genes, or a combination of any seventeen genes, or a combination of any eighteen genes chosen from, or a combination of any nineteen genes, or a combination of any twenty genes, or a combination of any twenty one genes, or a combination of any twenty two genes, or a combination of any twenty three genes, or a combination of any twenty four genes, or a combination of any twenty five genes, or a combination of any twenty six genes chosen from: ask, hom, metX, metY, metB, metH, metE, metF, zwf, metC, fprA, cysE, cysK, cysM, cysD, cysH, cysA, cysN, cysI, cysJ, cysX, cysZ, cysC, cysG, pyc and asd, where the genetic alterations result in overexpression of the genes in the combination. In other embodiments, microorganisms described herein include genetic alterations in a combination of any two, or any three, or any four, or any five, or any six, or any seven, or any eight, or all nine genes-chosen from ask^{fbr}, hom^{fbr}, metX, metY, metB, metH, metE, metF and zwf, where the genetic alterations lead to overexpression of the genes. For example, in some embodiments, microorganisms described herein include genetic alterations in a combination of any five genes chosen from ask^{fbr}, hom^{fbr}, metX, metY, metB, metH, mete, metF and zwf, where the genetic alterations lead to overexpression or constitutive expression of the any five genes. Microorganisms encompassed by this invention further include microorganisms that include genetic alterations in any six genes, or any seven genes, or any eight genes, or any nine genes chosen from ask^{fbr}, hom^{fbr}, metX, metY, metB, metH, metE, metF and zwf, where the genetic alterations lead to overexpression of the any six genes, or any seven genes, or any eight genes, or any nine genes. Microorganisms described herein also encompass microorganisms that have genetic alterations in two or more of genes chosen from mcbR, hsk, pepCK, metK and metQ, or any combinations thereof, where the genetic alterations lead to a decrease in expression of the genes. A decreased expression includes either lowering expression of the gene product encoded by a gene (e.g., mRNA and/or protein) and/or decreasing its activity (e.g., enzymatic activity of a protein encoded by the gene which is altered), or deleting/mutating the gene, such that no gene product is produced. In some embodiments, microorganisms include both overexpression of two or more genes that are favorable to methionine production (e.g., ask^{fbr}, hom^{fbr}, metX, metY, metB, metH, metE, metF and zwf) and decrease in expression of one or more genes, absence and/or lowering expression of which is beneficial for methionine production (e.g. mcbR, hsk, pepCK, metK and metQ).

The term “gene,” as used herein, includes a nucleic acid molecule (e.g., a DNA molecule or segment thereof) which is separated from another gene or other genes in an organism, by intergenic DNA (i.e., intervening or spacer DNA which naturally flanks the gene and/or separates genes in the chromosomal DNA of the organism). Alternatively, a gene may slightly overlap with another gene (e.g., the 3′ end of a first gene overlapping the 5′ end of a second gene), the overlapping genes separated from other genes by intergenic DNA. A gene may direct synthesis of an enzyme or another protein molecule (e.g., it may comprise coding sequences, for example, a contiguous open reading frame (ORF) which encodes a protein) or may itself be functional in the organism. A gene in an organism, may be clustered in an operon, as defined herein, the operon being separated from other genes and/or operons by the intergenic DNA. An “isolated gene,” as used herein, includes a gene which is essentially free of sequences which naturally flank the gene in the chromosomal DNA of the organism from which the gene is derived (i.e., is free of adjacent coding sequences that encode a second or distinct protein, adjacent structural sequences or the like) and optionally includes 5′ and 3′ regulatory sequences, for example promoter sequences and/or terminator sequences. In some embodiments, an isolated gene includes predominantly coding sequences for a protein (e.g., sequences which encode Corynebacterium proteins). In other embodiments, an isolated gene includes coding sequences for a protein (e.g., for a Corynebacterium protein) and adjacent 5′ and/or 3′ regulatory sequences from the chromosomal DNA of the organism from which the gene is derived (e.g., adjacent 5′ and/or 3′ Corynebacterium regulatory sequences). In some embodiments, an isolated gene contains less than about 10 kb, 5 kb, 2 kb, 1 kb, 0.5 kb, 0.2 kb, 0.1 kb, 50 bp, 25 bp, 10 bp, or fewer bp of nucleotide sequences which naturally flank the gene in the chromosomal DNA of the organism from which the gene is derived.

The terms “altered gene,” “genetic alteration,” “gene having an alteration” and “mutant gene,” as used interchangeably herein, refer to a gene having a nucleotide sequence which includes at least one modification (e.g., substitution, insertion, deletion) such that the polypeptide or protein encoded by the modified gene exhibits an activity that differs from the polypeptide or protein encoded by the wild-type nucleic acid molecule or gene. In some embodiments, a gene having an alteration or a mutant gene encodes a polypeptide or protein having an increased level or an increased activity as compared to the polypeptide or protein encoded by the wild-type gene, for example, when measured or assayed under similar conditions (e.g., assayed in microorganisms cultured at the same temperature and/or at the same concentration of an inhibitory compound). In other embodiments, a gene having an alteration or a mutant gene encodes a polypeptide or protein having a lower level or decreased activity as compared to the polypeptide or protein encoded by the wild-type gene, when measured or assayed under similar conditions. In some embodiments, a gene having an alteration or a mutant gene fails to encode a protein or polypeptide which is encoded by its wild-type counterpart. The terms “altered gene,” “mutant gene,” “gene having an alteration,” and “genetic alteration,” also include modifications in regulatory sequences for a gene or substitutions of regulatory sequences with heterologous sequences, including, but not limited to, promoters and/or enhancers, which result in an increase in, a decrease in, or absence of gene expression.

As used herein, terms “increased activity” and “increased enzymatic activity” refer to an activity that is at least 5% greater, or at least 5-10% greater, or at least 10-25% greater, or at least 25-50% greater, or at least 50-75% greater, or at least 75-100% greater than that of the polypeptide or protein encoded by the wild-type nucleic acid molecule or gene. Ranges intermediate to the above-recited values, e.g., 75-85%, 85-90%, 90-95%, are also intended to be encompassed herein. As used herein, “increased activity” and “increased enzymatic activity” also include an activity that is at least 1.25-fold, or at least 1.5-fold, or at least 2-fold, or at least 3-fold, or at least 4-fold, or at least 5-fold, or at least 10-fold, or at least 20-fold, or at least 50-fold, or at least 100-fold greater than the activity of the polypeptide or protein encoded by the wild-type gene.

Activity can be determined according to any well known assay for measuring activity of a particular protein of interest. Activity can be measured or assayed directly, for example, by measuring an activity of a protein in a crude cell extract or isolated or purified from a cell or microorganism. Alternatively, an activity can be measured or assayed within a cell or microorganism or in an extracellular medium. For example, assaying for a mutant can be accomplished by expressing the mutated or altered gene in a microorganism, for example, a mutant microorganism in which the enzyme is temperature-sensitive, and assaying the mutant gene for the ability to complement a temperature sensitive (Ts) mutant for enzymatic activity. A mutant or altered gene that encodes an “increased enzymatic activity” can be one that complements the Ts mutant more effectively than, for example, a corresponding wild-type gene. A mutant or altered gene that encodes a “reduced enzymatic activity” is one that complements the Ts mutant less effectively than, for example, a corresponding wild-type gene.

Without wishing to be bound by theory, it will be appreciated by a skilled artisan that even a single substitution in a nucleic acid or gene sequence (e.g., a base substitution that encodes an amino acid change in the corresponding amino acid sequence) can dramatically affect the activity of an encoded polypeptide or protein as compared to the corresponding wild-type polypeptide or protein. A mutant or altered gene (e.g., encoding a mutant or deregulated polypeptide or protein), as defined herein, is readily distinguishable from a nucleic acid or gene encoding a protein in that a mutant or altered gene encodes a protein or polypeptide having an altered level or activity, optionally observable as a different or distinct phenotype in a microorganism expressing the mutant gene or producing a mutant protein or polypeptide (i.e., a mutant or recombinant microorganism) as compared to a corresponding microorganism expressing the wild-type gene. By contrast, a protein encoded by a mutant gene can have an identical or substantially similar activity, optionally phenotypically indiscernible when produced in a microorganism, as compared to a corresponding microorganism expressing the wild-type gene. Accordingly it is not, for example, only the degree of sequence identity between nucleic acid molecules, genes, protein or polypeptides that may serve to distinguish between homologs and mutants, rather it is the level or activity of the encoded protein or polypeptide that distinguishes between homologs and mutants: homologs having, for example, low (e.g., 30-50% sequence identity) sequence identity yet having substantially equivalent functional activities, and mutants, for example sharing 99% sequence identity yet having dramatically different or altered functional activities.

In some embodiments, a gene having a mutation or a mutant gene encodes a polypeptide or protein having a reduced or increased activity as compared to the polypeptide or protein encoded by the wild-type gene, for example, when assayed under similar conditions (e.g., assayed in microorganisms cultured at the same temperature or in the presence of the same concentration of an inhibitor). A mutant gene may also encode no polypeptide or have a reduced level of production of the wild-type polypeptide.

As used herein, terms “reduced activity” and “reduced enzymatic activity” refer to an activity that is at least 5% less, or at least 5-10% less, or at least 10-25% less, or at least 25-50%, or at least 50-75%, or at least 75-100% less than that of the polypeptide or protein encoded by the wild-type nucleic acid molecule or gene. Ranges intermediate to the above-recited values, e.g., 75-85%, 85-90%, 90-95%, are also intended to be encompassed herein. As used herein, a “reduced activity” or “reduced enzymatic activity” can also include an activity that has been deleted or “knocked out” (e.g., approximately 100% less activity than that of the polypeptide or protein encoded by the wild-type nucleic acid molecule or gene).

In some embodiments, recombinant microorganisms described herein comprise deregulation of at least two proteins, or at least three proteins, or at least four proteins, or at least five proteins, or at least six proteins, or at least seven proteins, or at least eight proteins, or at least nine proteins, or at least ten proteins, or at least ten proteins, or at least eleven proteins, or at least twelve proteins, or at least thirteen proteins, or at least fourteen proteins, or at least fifteen proteins, or at least sixteen proteins, or at least seventeen proteins, or at least eighteen proteins, or at least nineteen proteins, or at least twenty proteins, or at least twenty one proteins, or at least twenty two proteins, or at least twenty three proteins, or at least twenty four proteins, or at least twenty five proteins, or at least twenty six proteins, or at least twenty seven proteins, or at least twenty eight proteins, or at least twenty nine proteins, or at least thirty proteins, or at least thirty one proteins, or at least thirty two proteins, or at least thirty three proteins, or at least thirty four proteins chosen from Aspartate kinase, Homoserine dehydrogenase, Homoserine acetyltransferase, O-Succinylhomoserine sulfyhydralase, Cystationine γ synthase, Cystathionine β-lyase, O-Acetylhomoserine sulfhydralase, Vitamin B12-dependent methionine synthase, Vitamin B12-independent methionine synthase, N5,10-methylene-tetrahydrofolate reductase, S-adenosylmethionine synthase, Methionine import protein, NADP-ferredoxin reductase, Aspartate semialdehyde dehydrogenase, Cystathionine beta synthetase, Sulfite reductase (subunits 1 or 2 or both), Serine acetyltransferase, O-acetylserine (thiol)-lyase A, Sulfate adenylyltransferase (subunit 1 or 2 or both), Phosphoadenosine phosphosulfate reductase, Gamma-cystathionase, APS kinase, APS reductase, Glucose-6-phosphate dehydrogenase, Pyruvate carboxylase, Homoserine kinase, Uroporphyrinogen III synthase, APS phosphatase, Sulfate transporter, Accessory role sulfite reduction, Threonine dehydrogenase, TetR-type transcriptional regulator of sulfur metabolism and Phosphoenolpyruvate carboxykinase.

In some embodiments, recombinant microorganisms described herein comprise two or more, or three or more, or four or more, or five or more, or six or more, or seven or more, or eight or more, or nine or more, or ten or more, or eleven or more, or twelve or more, or thirteen or more, or fourteen or more, or fifteen or more, or sixteen or more, or seventeen or more, or eighteen or more, or nineteen or more, or twenty or more, or twenty one or more, or twenty two or more, or twenty three or more, or twenty four or more, or twenty five or more, or twenty six or more, or twenty seven or more deregulated proteins chosen from Aspartate kinase, Homoserine dehydrogenase, Homoserine acetyltransferase, O-Succinyl homoserine sulfyhydralase, Homoserine succinyltransferase, Cystationine γ synthase, Cystathionine β-lyase, O-Acetylhomoserine sulfhydralase, Vitamin B12-dependent methionine synthase, Vitamin B12-independent methionine synthase, N5,10-methylene-tetrahydrofolate reductase, NADP-ferredoxin reductase, Aspartate semialdehyde dehydrogenase, Sulfite reductase (subunit 1 or 2 or both), Serine O-acetyltransferase, O-acetylserine (thiol)-lyase A, Sulfate adenylyltransferase (subunit 1 or 2 or both), APS kinase, APS reductase, Phosphoadenosine phosphosulfate reductase, Gamma-cystathionase, Glucose-6-phosphate dehydrogenase, Uroporphyrinogen III synthase, Sulfate transporter, Accessory role sulfite reduction, and Pyruvate decarboxylase, where the deregulated proteins are expressed at a level greater than and/or have a greater activity relative to the expression or activity in a microorganism that includes a wild-type counterpart of the protein or which does not express the protein.

In some embodiments, recombinant microorganisms described herein comprise two or more deregulated proteins chosen from Methionine import protein, S-Adenosylmethionine synthase, Cystathionine beta synthetase, APS phosphates, Homoserine kinase, TetR-type transcriptional regulator of sulfur metabolism, phosphoenolpyruvate carboxykinase and threonine dehydratase, where the two or more deregulated proteins are expressed at a level lower than and/or have a decreased activity relative to the expression or activity in a microorganism that includes a wild-type counterpart of the protein.

It is understood that a deregulated protein may be expressed at a level higher than level of the wild-type protein which and/or it has a higher activity relative to the wild-type protein. Alternatively, it may be expressed at a level lower than level of the wild-type protein and/or have a lower or decreased activity relative to the wild-type protein. In some instances, a deregulated protein is constitutively expressed and in other instances, a deregulated protein is not expressed at all or has lost its enzymatic activity. In some embodiments, a protein that is deregulated is an enzyme in the methionine biosynthetic pathway. In other embodiments, a protein that is deregulated is an enzyme in the cysteine biosynthetic pathway. In yet other embodiments, a protein that is deregulated is a transcriptional repressor or activator of genes in the methionine biosynthetic pathway and/or the cysteine biosynthetic pathway. In certain instances, a protein is deregulated such that it is feedback resistant. A deregulated protein is usually expressed by a genetically altered or modified gene in a microorganism.

Recombinant microorganisms described herein encompass microorganisms that have been genetically modified or altered in a way that they express two or more, or three or more, or four or more, or five or more, or six or more, or seven or more, or eight or more, or nine or more, or ten or more, or eleven or more, or twelve or more proteins, or thirteen or more, or fourteen or more, or fifteen or more, or sixteen or more, or seventeen or more, or eighteen or more, or nineteen or more, or twenty or more, or twenty one or more, or twenty two or more, or twenty three or more, or twenty four or more, or twenty five or more, or twenty six or more, or twenty seven or more, or twenty eight or more, or twenty nine or more, or thirty or more, or thirty one or more, or thirty two or more, or thirty three or more, or thirty four or more proteins at a level which is higher or lower than the level of protein produced in a microorganism which has not been genetically modified or altered. For example, in some embodiments, recombinant microorganisms produce five or more proteins with an activity (e.g., enzymatic activity) which is greater or lower than the activity of the protein in a microorganism which has not been genetically modified or altered.

In some embodiments, recombinant microorganisms described herein include, for example, a combination of genes that have been altered, where the level of methionine produced is greater than the sum of methionine levels produced in presence of each individual gene alteration in the combination (i.e., alteration of a combination of genes has a greater than additive, or synergistic, effect on methionine production). For example, microorganisms encompassed by this invention include microorganisms including two or more altered genes, where the level of methionine produced is greater than the sum of levels of methionine produced in presence of each individual altered gene. Accordingly, a synergistic effect of altering two or more, or three or more, or five or more, or six or more, or seven or more, or eight or more, or nine or more, or ten or more genes, for example, can be measured for any combination of the various genes described herein. In some embodiments, microorganisms including a combination of altered genes produce methionine, for example, at a level which is at least 1-2% greater, or at least 3-5% greater, or at least 5-10% greater, or at least 10-20% greater, or at least 20-30% greater, or at least 30-40% greater, or at least 40-50% greater, or at least 50-60% greater, or at least 60-70% greater, or at least 70-80% greater, or at least 80-90% greater, or at least 90-95% greater than the sum of methionine levels produced in presence of each individual altered gene, or in the presence of no alterations.

In some embodiments, level of methionine produced by microorganisms including a combination of altered genes is at least 2-fold, or at least 2.5-fold, or at least 3-fold, or at least 3.5-fold, or at least 4-fold, or at least 4.5-fold, or at least 5-fold, or at least 10-fold, or at least 15-fold, or at least 20-fold, or at least 25-fold, or at least 30-fold, or at least 35-fold, or at least 40-fold, or at least 45-fold, or at least 50-fold, or at least 100-fold higher than the sum of levels of methionine produced in presence of each individual altered gene, or in presence of no alterations.

In yet other embodiments, amount of methionine produced by a microorganism under suitable fermentation conditions, including a combination of altered genes, is at least 5 g, or at least 7 g, or at least 8 g, or at least 9 g, or at least 10 g, or at least 11 g, or at least 12 g, or at least 13 g, or at least 14 g, or at least 15 g, or at least 16 g, or at least 17 g, or at least 18 g, or at least 19 g, or at least 20 g, or at least 25 g, or at least 30 g, or at least 40 g, or at least 50 g greater per liter relative to the sum of amounts produced by a microorganism in the presence of each individual altered gene, or in presence of no gene alterations.

The level of methionine produced by microorganisms described herein can be easily measured using one or more assays described herein.

In some embodiments, “recombinant microorganisms” encompassed by this invention have a deregulated cysteine biosynthetic pathway. The phrase “microorganism having a deregulated cysteine biosynthetic pathway” includes a microorganism having an alteration or modification in at least two, or at least three, or at least four, or at least five, or at least six, or at least seven, or at least eight, or at least nine, or at least ten, or at least eleven, or at least twelve, or at least thirteen genes encoding enzymes of the cysteine biosynthetic pathway or having an alteration or modification in an operon including genes encoding enzymes of the cysteine biosynthetic pathway. In some embodiments, microorganisms having a deregulated cysteine biosynthetic pathway described herein are genetically engineered to include genetic alterations in at least two genes chosen from cysJ, cysA, cysE, cysK, cysM, cysD, cysI, cysN, cysG, cysC, cysX cysZ, and cysH, such that the genes are overexpressed. In some embodiments, microorganisms having a deregulated cysteine biosynthetic pathway are genetically engineered to include genetic alteration(s) in cysQ and/or cysY, thereby to decrease the expression of one or both genes. In yet other embodiments, recombinant microorganisms with a deregulated cysteine biosynthetic pathway include a combination of genetic alterations in at least two, or at least three, or at least four, or at least five, or at least six genes chosen from cysJ, cysA, cysE, cysK, cysM, cysD, cysI, cysN, cysG, cysC, cysY, cysX, cysZ, cysH and cysQ.

Further featured herein are mutant microorganisms. As used herein, the term “mutant microorganism” includes a recombinant microorganism that has been genetically engineered to express a mutated or altered gene or protein that is normally or naturally expressed by the microorganism. For example, in some embodiments a mutant microorganism expresses a mutated gene or protein such that the microorganism exhibits an altered, modified or different phenotype. In other embodiments, a mutant microorganism is altered or engineered such that a gene has been deleted (i.e., the protein encoded by the gene is not produced).

In some embodiments, a recombinant microorganism described herein is a Gram positive organism (e.g., a microorganism which retains basic dye, for example, crystal violet, due to the presence of a Gram-positive wall surrounding the microorganism). In other embodiments, a recombinant microorganism is a microorganism belonging to a genus chosen from Bacillus, Cornyebacterium, Lactobacillus, Lactococci and Streptomyces. In yet other embodiments, a recombinant microorganism belongs to the genus Cornyebacterium and in some embodiments, a recombinant microorganism is chosen from Cornyebacterium glutamicum.

In some embodiments, a recombinant microorganism is a Gram negative (excludes basic dye) organism. In other embodiments, a recombinant microorganism is a microorganism belonging to a genus chosen from Salmonella, Escherichia, Klebsiella, Serratia, and Proteus. In yet other embodiments, a recombinant microorganism is a yeast such as chosen from the genus Saccharomyces, Kluyveromyces, Pichia, Candida, Schizosaccharomyces, etc. (e.g., S. cerevisiae), or an Archaea.

An important aspect encompassed by this invention includes culturing recombinant microorganisms described herein under suitable conditions, such that methionine is produced. The term “culturing” includes maintaining and/or growing a living microorganism described herein (e.g., maintaining and/or growing a culture or strain). In some embodiments, a microorganism is cultured in liquid media. In other embodiments, a microorganism is cultured in solid media or semi-solid media. In yet other embodiments, a microorganism is cultured in media (e.g., a sterile, liquid medium) comprising nutrients essential or beneficial to the maintenance and/or growth of the microorganism (e.g., carbon sources or carbon substrate, for example complex carbohydrates such as bean or grain meal, starches, sugars, sugar alcohols, hydrocarbons, oils, fats, fatty acids, organic acids and alcohols; nitrogen sources, for example, vegetable proteins, peptones, peptides and amino acids derived from grains, beans and tubers, proteins, peptides and amino acids derived form animal sources such as meat, milk and animal byproducts such as peptones, meat extracts and casein hydrolysates; inorganic nitrogen sources such as urea, ammonium sulfate, ammonium chloride, ammonium nitrate and ammonium phosphate; phosphorus sources, for example, phosphoric acid, sodium and potassium salts thereof; trace elements, for example, magnesium, iron, manganese, calcium, copper, zinc, boron, nickel, molybdenum, and/or cobalt salts; as well as growth factors such as amino acids, vitamins, growth promoters and the like).

In some instances, microorganisms described herein are cultured under controlled pH. The term “controlled pH” includes any pH which results in production of methionine. In some embodiments, microorganisms are cultured at a pH of about 7. In other embodiments, microorganisms are cultured at a pH of between 6.0 and 8.5. The desired pH may be maintained by any number of methods known to those skilled in the art.

Also, in some instances, microorganisms described herein are cultured under controlled aeration. The term “controlled aeration” includes sufficient aeration (e.g., oxygen) which results in production of methionine. In some embodiments, aeration is controlled by regulating oxygen levels in the culture, for example, by regulating the amount of oxygen dissolved in culture media. For example, aeration of the culture may be controlled by agitating the culture. Agitation may be provided by a propeller or similar mechanical agitation equipment, by revolving or shaking the growth vessel (e.g., fermentor) or by various pumping equipment. Aeration may be further controlled by the passage of sterile air or oxygen through the medium (e.g., through the fermentation mixture). Also, microorganisms are cultured without excess foaming (e.g., via addition of antifoaming agents).

Additionally, microorganisms described herein may be cultured under controlled temperatures. The term “controlled temperature” includes any temperature which results in production of methionine. In some embodiments, controlled temperature is set to a specified temperature, for example, between 15° C. and 95° C., between 15° C. and 70° C., between 20° C. and 55° C., between 30° C. and 45° C., or between 30° C. and 50° C., or between 28° C. and 37° C.

Microorganisms can be cultured (e.g., maintained and/or grown) in liquid media and preferably are cultured, either continuously or intermittently, by conventional culturing methods such as standing culture, test tube culture, shaking culture (e.g., rotary shaking culture, shake flask culture, etc.), aeration spinner culture, or fermentation. In some embodiments, microorganisms are cultured in shake flasks. In yet other embodiments, microorganisms are cultured in a fermentor (e.g., in a fermentation process). Fermentation processes include, but are not limited to, batch, fed-batch and continuous methods of fermentation. The terms “batch process” and “batch fermentation” refer to a closed system in which the composition of media, nutrients, supplemental additives and the like is set at the beginning of the fermentation and not subject to alteration during the fermentation; however, attempts may be made to control such factors as pH and oxygen concentration to prevent excess media acidification and/or microorganism death. The terms “fed-batch process” and “fed-batch” fermentation refer to a batch fermentation with the exception that one or more substrates or supplements are added (e.g., added in increments or continuously) as the fermentation progresses. The terms “continuous process” and “continuous fermentation” refer to a system in which a defined fermentation media is added continuously to a fermentor and an equal amount of used or “conditioned” media is simultaneously removed, for example, for recovery of the desired product (e.g., methionine). A variety of such processes have been developed and are well-known in the art.

Microorganisms described herein may be cultured continuously or batchwise or in a fed batch or repeated fed batch process to produce methionine. An overview of known cultivation methods can be found in the textbook by Chmiel (Bioprozelitechnik 1. Einfiihrung in die Bioverfahrenstechnik (Gustav Fischer Verlag, Stuttgart, 1991)) or in the textbook by Storhas (Bioreaktoren und periphere Einrichtungen (Vieweg Verlag, Braunschweig/Wiesbaden, 1994)). A culture medium to be used must meet the requirements of the particular strains in a suitable manner. Descriptions of culture media for various microorganisms are contained in the handbook “Manual of Methods for General Bacteriology” of the American Society for Bacteriology (Washington D.C., USA, 1981).

The phrases “culturing under conditions such that a desired compound (e.g., methionine) is produced” and “suitable conditions” refers to maintaining and/or growing microorganisms under conditions (e.g., temperature, pressure, pH, duration, etc.) appropriate or sufficient to obtain production of the desired compound or to obtain desired yields of the particular compound being produced. For example, microorganisms are cultured under suitable conditions for a time sufficient to produce the desired amount of methionine. In some embodiments, microorganisms are cultured for a time sufficient to substantially reach a maximal production of methionine. In some embodiments, microorganisms are cultured for about 12 to 24 hours. In other embodiments, microorganisms are cultured for about 24 to 36 hours, about 36 to 48 hours, about 48 to 72 hours, about 72 to 96 hours, about 96 to 120 hours, about 120 to 144 hours, or for a duration greater than 144 hours. In yet other embodiments, culturing is continued for a time sufficient to reach desirable production yields of methionine, for example, microorganisms are cultured such that at least about 7 to 10 g/l, or at least 10 to 15 g/l, or at least about 15 to 20 g/l, or at least about 20 to 25 g/l, or at least about 25 to 30 g/l, or at feast about 30 to 35 g/l, or at least about 35 to 40 g/l, or at least about 40 to 50 g/l methionine is produced. In some embodiments, the amount of methionine produced by recombinant microorganisms encompassed by this invention is at least 16 g/l. In yet other embodiments, the amount of methionine produced under suitable fermentation conditions by recombinant microorganisms described herein is at least 17 g/l. In yet other embodiments, microorganisms are cultured under conditions such that a preferred yield of methionine, for example, a yield within a range set forth above, is produced in about 24 hours, in about 36 hours, in about 48 hours, in about 72 hours, or in about 96 hours.

The methodologies described herein can further include a step of recovering a desired compound (e.g., methionine). The term “recovering” a desired compound (e.g., methionine) refers to extracting, harvesting, isolating or purifying the compound from culture media. Recovering the compound can be performed according to any conventional isolation or purification methodology known in the art including, but not limited to, centrifugation, evaporation, treatment with a conventional resin (e.g., anion or cation exchange resin, non-ionic adsorption resin, etc.), treatment with a conventional adsorbent (e.g., activated charcoal, silicic acid, silica gel, cellulose, alumina, etc.), alteration of pH, solvent extraction (e.g., with a conventional solvent such as an alcohol, ethyl acetate, hexane and the like), dialysis, filtration, concentration, crystallization, recrystallization, pH adjustment, lyophilization and the like. For example, methionine can be recovered from culture media by first removing the microorganisms from the culture.

In some embodiments, methionine is “extracted,” “isolated” or “purified” such that it is substantially free of other components (e.g., free of media components and/or fermentation byproducts). The phrase “substantially free of other components” refers to preparations of desired compound, for example, methionine, in which methionine is separated (e.g., purified or partially purified) from media components or fermentation byproducts of the culture from which it is produced. In some embodiments, a preparation has greater than about 80% (by dry weight) of methionine (e.g., less than about 20% of other media components or fermentation byproducts), or greater than about 90% of methionine (e.g., less than about 10% of other media components or fermentation byproducts), or greater than about 95% of methionine (e.g., less than about 5% of other media components or fermentation byproducts), or greater than about 98-99% methionine (e.g., less than about 1-2% other media components or fermentation byproducts).

In an alternative embodiment, methionine is not purified from the microorganism, for example, when the microorganism is biologically non-hazardous (e.g., safe). For example, the entire culture (or culture supernatant) can be used as a source of product (e.g., crude product). In one embodiment, the culture (or culture supernatant) is used without modification. In another embodiment, the culture (or culture supernatant) is concentrated. In yet another embodiment, the culture (or culture supernatant) is dried or lyophilized.

This invention further encompasses biotransformation processes which feature various recombinant microorganisms described herein. The term “biotransformation process,” also referred to herein as “bioconversion processes,” includes biological processes which results in the production (e.g., transformation or conversion) of appropriate substrates and/or intermediate compounds into a desired product (e.g., methionine).

Microorganism(s) and/or enzymes used in biotransformation reactions are in a form that allows them to perform their intended function (e.g., producing a desired compound). Such microorganisms can be whole cells, or can be only those portions of a cell (for example genes and/or enzymes) necessary to obtain the desired end result. These microorganisms can be suspended (e.g., in an appropriate solution such as buffered solutions or media), rinsed (e.g., rinsed free of media from culturing the microorganism), acetone-dried, immobilized (e.g., with polyacrylamide gel or k-carrageenan or on synthetic supports, for example, beads, matrices and the like), fixed, cross-linked or permeabilized (e.g., have permeabilized membranes and/or walls such that compounds, for example, substrates, intermediates or products can more easily pass through said membrane or wall).

This invention further encompasses recombinant nucleic acid molecules (e.g., recombinant DNA molecules) that include genes described herein (e.g. isolated genes) including Corynebacterium genes, such as, for example, Corynebacterium glutamicum genes and more specifically, Corynebacterium glutamicum methionine biosynthetic genes and Corynebacterium glutamicum cysteine biosynthetic genes. The term “recombinant nucleic acid molecule” refers to a nucleic acid molecule (e.g., a DNA molecule) that has been altered, modified or engineered such that it differs in nucleotide sequence from the native or natural nucleic acid molecule from which the recombinant nucleic acid molecule was derived (e.g., by addition, deletion or substitution of one or more nucleotides). In some embodiments, a recombinant nucleic acid molecule (e.g., a recombinant DNA molecule) includes an isolated gene operably linked to regulatory sequences. The phrase “operably linked to regulatory sequence(s)” means that the nucleotide sequence of the gene of interest is linked to the regulatory sequence(s) in a manner which allows for expression (e.g., enhanced, increased, constitutive, basal, attenuated, decreased or repressed expression) of the gene, for example, expression of a gene product encoded by the gene (e.g., when the recombinant nucleic acid molecule is included in a recombinant vector, as defined herein, and is introduced into a microorganism).

The term “heterologous nucleic acid” is used herein to refer to nucleic acid sequences not typically present in a microorganism. Such nucleic acid sequences also include nucleic acid sequences present in a microorganism, but not in a genetic location where they are normally found in the microorganism. Similarly, the term “heterologous gene” can include a gene not present in a wild-type microorganism. Heterologous nucleic acids and heterologous genes generally comprise recombinant nucleic acid molecules. Heterologous nucleic acid or heterologous genes may or may not include modifications (e.g., by addition, deletion or substitution of one or more nucleotides).

Also encompassed by this invention are homologs of the various genes and proteins described herein. A “homolog,” in reference to a gene refers to a nucleotide sequence that is substantially identical over at least part of the gene or to its complementary strand or a part thereof, provided that the nucleotide sequence encodes a protein that has substantially the same activity/function as the protein encoded by the gene which it is a homolog of. Homologs of the genes described herein can be identified by percent identity between amino acid or nucleotide sequences for putative homologs and the sequences for the genes or proteins encoded by them (e.g. nucleotide sequences for Corynebacterium glutamicum genes ask, hom, metX, metY, metB, metH, metE, metF, zwf, metC, metK, metQ, cysJ, cysE, cysK, cysM, cysD, cysH, cysA, mcbR, hsk and pepCK, or their complementary strands). Percent identity may be determined, for example, by visual inspection or by using various computer programs known in the art or as described herein. For example, percent identity of two nucleotide sequences can be determined by comparing sequence information using the GAP computer program described by Devereux et al. (1984) Nucl. Acids. Res., 12:387 and available from the University of Wisconsin Genetics Computer Group (UWGCG). Percent identity can also be determined by aligning two nucleotide sequences using the Basic Local Alignment Search Tool (BLAST™) program (as described by Tatusova et al. (1999) FEMS Microbiol. Lett., 174:247. For example, for nucleotide sequence alignments using the BLAST™ program, the default settings are as follows: reward for match is 2, penalty for mismatch is −2, open gap and extension gap penalties are 5 and 2 respectively, gap.times.dropoff is 50, expect is 10, word size is 11, and filter is OFF.

As used herein, the terms “homology” and “homologous” are not limited to designate proteins having a theoretical common genetic ancestor, but includes proteins which may be genetically unrelated that have, none the less, evolved to perform similar functions and/or have similar structures. Functional homology to the various proteins described herein also encompasses proteins that have an activity of the corresponding protein it is a homolog of. For proteins to have functional homology, it is not required that they have significant identity in their amino acid sequences, but, rather, proteins having functional homology are so defined by having similar or identical activities, e.g., enzymatic activities. Similarly, proteins with structural homology are defined as having analogous tertiary (or quaternary) structure and do not necessarily require amino acid identity or nucleic acid identity for the genes encoding them. In certain circumstances, structural homologs may include proteins which maintain structural homology only at the active site or binding site of the protein.

In addition to structural and functional homology, the present invention further encompasses proteins having amino acid identity to the various proteins and enzymes described herein. To determine the percent identity of two amino acid sequences, the sequences are aligned for optimal comparison purposes (e.g., gaps can be introduced in the amino acid sequence of one protein for optimal alignment with the amino acid sequence of another protein). The amino acid residues at corresponding amino acid positions are then compared. When a position in one sequence is occupied by the same amino acid residue as the corresponding position in the other, then the molecules are identical at that position. The percent identity between the two sequences is a function of the number of identical positions shared by the sequences (i.e., % identity=# of identical positions/total # of positions multiplied by 100).

In some embodiments, nucleic acid and amino acid sequences of molecules described herein comprise a nucleotide sequence or amino acid sequence which hybridizes to or is at least about 50%, 60%, 70%, 80%, 90%, 95%, 96%, 97%, 98%, 99% or more identical to a nucleic acid or amino acid sequence described herein.

Techniques useful for the genetic engineering of the proteins described herein to produce enzymes with improved or modified characteristics are also described herein. For example, it is well within the teachings available in the art to modify a protein such that the protein has increased or decreased substrate binding affinity. It also may be advantageous, and within the teachings of the art, to design a protein which has increased or decreased enzymatic rates. Particularly for multifunctional enzymes, it may be useful to differentially fine tune the various activities of a protein to perform optimally under specified circumstances. Further the ability to modulate an enzyme's sensitivity to feedback inhibition (e.g., by methionine) may be accomplished through selective change of amino acids involved in binding or coordination of methionine or other cofactors which may be involved in negative or positive feedback. Further, genetic engineering encompasses events associated with the regulation of expression at the levels of both transcription and translation. For example, when a complete or partial operon is used for cloning and expression, regulatory sequences e.g. promoter or enhancer sequences of the gene may be modified such that they yield desired levels of transcription.

A “homolog” of any of the genes described herein can also be identified by an activity of the protein encoded by the homolog. For example, such a homolog can complement a mutation in the gene which it is a homolog of.

The term “regulatory sequence” refers to nucleic acid sequences that affect (e.g., modulate or regulate) expression of other nucleic acid sequences (i.e., genes). In some embodiments, a regulatory sequence is included in a recombinant nucleic acid molecule in a similar or identical position and/or orientation relative to a particular gene of interest as is observed for the regulatory sequence and gene of interest as it appears in nature, e.g., in a native position and/or orientation. For example, a gene of interest can be included in a recombinant nucleic acid molecule operably linked to a regulatory sequence which accompanies or is adjacent to the gene of interest in the natural organism (e.g., operably linked to “native” regulatory sequences (e.g., to the “native” promoter). Alternatively, a gene of interest can be included in a recombinant nucleic acid molecule operably linked to a regulatory sequence that accompanies or is adjacent to another (e.g., a different) gene in the natural organism. Alternatively, a gene of interest can be included in a recombinant nucleic acid molecule operably linked to a regulatory sequence from another organism. For example, regulatory sequences from other microbes (e.g., other bacterial regulatory sequences, bacteriophage regulatory sequences and the like) can be operably linked to a particular gene of interest.

In one embodiment, a regulatory sequence is a non-native or non-naturally-occurring sequence (e.g., a sequence which has been modified, mutated, substituted, derivatized, deleted including sequences which are chemically synthesized). Examples of regulatory sequences include promoters, enhancers, termination signals, anti-termination signals and other expression control elements (e.g., sequences to which repressors or inducers bind and/or binding sites for transcriptional and/or translational regulatory proteins, for example, in the transcribed mRNA). Such regulatory sequences are described, for example, in Sambrook, J., Fritsh, E. F., and Maniatis, T. Molecular Cloning: A Laboratory Manual. 2^{nd}, ed., Cold Spring Harbor Laboratory, Cold Spring Harbor Laboratory Press, Cold Spring Harbor, N.Y., 1989, and in Patek, M. et al, (2003) Journal of Biotechnology 104:311-323. Regulatory sequences include those which direct constitutive expression of a nucleotide sequence in a microorganism (e.g., constitutive promoters and strong constitutive promoters), those that direct inducible expression of a nucleotide sequence in a microorganism (e.g., inducible promoters, for example, xylose inducible promoters) and those that attenuate or repress expression of a nucleotide sequence in a microorganism (e.g., attenuation signals or repressor sequences). It is also within the scope of this invention to regulate expression of a gene of interest by removing or deleting regulatory sequences. For example, sequences involved in the negative regulation of transcription can be removed such that expression of a gene of interest is enhanced.

In some embodiments, a recombinant nucleic acid molecule described herein includes a nucleic acid sequence or gene that encodes at least one bacterial gene product (e.g., a methionine biosynthetic enzyme) operably linked to a promoter or promoter sequence. Promoters featured herein include, but are not limited to, Corynebacterium promoters and/or bacteriophage promoters (e.g., bacteriophage which infect Corynebacterium or other bacteria). For example, in some embodiments, a promoter is a Corynebacterium promoter, such as a strong, Corynebacterium promoter (e.g., a promoter associated with a biochemical housekeeping gene in Corynebacterium). In other embodiments, a promoter is a bacteriophage promoter. Additional promoters for use in Gram positive microorganisms include, but are not limited to, superoxide dismutase, groEL, groES, elongation factor Tu, amy and SPO1 promoters, such as P₁₅ and P₂₆— Examples of promoters for use in Gram negative microorganisms include, but are not limited to, cos, tac, trp, tet, trp-tet, lpp, lac, lpp-lac, lacIQ, T7, T5, T3, gal, trc, ara, SP6, λ-PR and λ-PL.

In some embodiments, a recombinant nucleic acid includes a terminator sequence or terminator sequences (e.g., transcription terminator sequences). The term “terminator sequences” includes regulatory sequences that serve to terminate transcription of mRNA. Terminator sequences (or tandem transcription terminators) can further serve to stabilize mRNA (e.g., by adding structure to mRNA), for example, against nucleases.

In some embodiments, a recombinant nucleic acid molecule includes sequences that allow for detection of the vector containing said sequences (i.e., detectable and/or selectable markers), for example, genes that encode antibiotic resistance sequences or that overcome auxotrophic mutations, for example, trpC, drug markers, fluorescent markers, and/or colorimetric markers (e.g., lacZ/β-galactosidase). In yet other embodiments, a recombinant nucleic acid molecule includes an artificial ribosome binding site (RBS) or a sequence that gets transcribed into an artificial RBS. The term “artificial ribosome binding site (RBS)” includes a site within an mRNA molecule (e.g., coded within DNA) to which a ribosome binds (e.g., to initiate translation) which differs from a native RBS (e.g. a RBS found in a naturally-occurring gene) by at least one nucleotide. Preferred artificial RBSs include about 5-6, 7-8, 9-10, 11-12, 13-14, 15-16, 17-18, 19-20, 21-22, 23-24, 25-26, 27-28, 29-30 or more nucleotides of which about 1-2, 3-4, 5-6, 7-8, 9-10, 11-12, 13-15 or more differ from the native RBS (e.g., the native RBS of a gene of interest).

Further encompassed by this invention are vectors (e.g., recombinant plasmids and bacteriophages) that include nucleic acid molecules (e.g., genes or recombinant nucleic acid molecules comprising said genes) as described herein. The term “recombinant vector” includes a vector (e.g., plasmid, phage, phasmid, virus, cosmid, fosmid, or other purified nucleic acid vector) that has been altered, modified or engineered such that it contains greater, fewer or different nucleic acid sequences than those included in the native or natural nucleic acid molecule from which the recombinant vector was derived. For example, a recombinant vector includes a biosynthetic enzyme-encoding gene or recombinant nucleic acid molecule including said gene, operably linked to regulatory sequences, for example, promoter sequences, terminator sequences and/or artificial ribosome binding sites (RBSs), as defined herein. In some embodiments, a recombinant vector includes sequences that enhance replication in bacteria (e.g., replication-enhancing sequences). In some embodiments, replication-enhancing sequences function in E. coli or C. glutamicum. In other embodiments, replication-enhancing sequences are derived from plasmids including, but not limited to, pBR322, pACYC177, pACYC184 and pSC101.

In some embodiments, a recombinant vector of the present invention includes antibiotic resistance sequences. The term “antibiotic resistance sequences” includes sequences which promote or confer resistance to antibiotics on the host organism (e.g., Corynebacterium). In some embodiments, antibiotic resistance sequences are chosen from: cat (chloramphenicol resistance) sequences, tet (tetracycline resistance) sequences, erm (erythromycin resistance) sequences, neo (neomycin resistance) sequences, kan (kanamycin resistance) sequences and spec (spectinomycin resistance) sequences. Recombinant vectors can further include homologous recombination sequences (e.g., sequences designed to allow recombination of the gene of interest into the chromosome of the host organism). It will further be appreciated by one of skill in the art that the design of a vector can be tailored depending on such factors as the choice of microorganism to be genetically engineered, the level of expression of gene product desired and the like.

“Campbell in,” as used herein, refers to a transformant of an original host cell in which an entire circular double stranded DNA molecule (for example a plasmid) has integrated into a chromosome by a single homologous recombination event (a cross in event), and that effectively results in the insertion of a linearized version of said circular DNA molecule into a first DNA sequence of the chromosome that is homologous to a first DNA sequence of the said circular DNA molecule. “Campbelled in” refers to the linearized DNA sequence that has been integrated into the chromosome of a “Campbell in” transformant. A “Campbell in” contains a duplication of the first homologous DNA sequence, each copy of which includes and surrounds a copy of the homologous recombination crossover point. The name comes from Professor Alan Campbell, who first proposed this kind of recombination.

“Campbell out,” as used herein, refers to a cell descending from a “Campbell in” transformant, in which a second homologous recombination event (a cross out event) has occurred between a second DNA sequence that is contained on the linearized inserted DNA of the “Campbelled in” DNA, and a second DNA sequence of chromosomal origin, which is homologous to the second DNA sequence of said linearized insert, the second recombination event resulting in the deletion (jettisoning) of a portion of the integrated DNA sequence, but, importantly, also resulting in a portion (this can be as little as a single base) of the integrated Campbelled in DNA remaining in the chromosome, such that compared to the original host cell, the “Campbell out” cell contains one or more intentional changes in the chromosome (for example, a single base substitution, multiple base substitutions, insertion of a heterologous gene or DNA sequence, insertion of an additional copy or copies of a homologous gene or a modified homologous gene, or insertion of a DNA sequence comprising more than one of these aforementioned examples listed above).

A “Campbell out” cell or strain is usually, but not necessarily, obtained by a counter-selection against a gene that is contained in a portion (the portion that is desired to be jettisoned) of the “Campbelled in” DNA sequence, for example the Bacillus subtilis sacB gene, which is lethal when expressed in a cell that is grown in the presence of about 5% to 10% sucrose. Either with or without a counter-selection, a desired “Campbell out” cell can be obtained or identified by screening for the desired cell, using any screenable phenotype, such as, but not limited to, colony morphology, colony color, presence or absence of antibiotic resistance, presence or absence of a given DNA sequence by polymerase chain reaction, presence or absence of an auxotrophy, presence or absence of an enzyme, colony nucleic acid hybridization, antibody screening, etc. The term “Campbell in” and “Campbell out” can also be used as verbs in various tenses to refer to the method or process described above.

It is understood that the homologous recombination events that leads to a “Campbell in” or “Campbell out” can occur over a range of DNA bases within the homologous DNA sequence, and since the homologous sequences will be identical to each other for at least part of this range, it is not usually possible to specify exactly where the crossover event occurred. In other words, it is not possible to specify precisely which sequence was originally from the inserted DNA, and which was originally from the chromosomal DNA. Moreover, the first homologous DNA sequence and the second homologous DNA sequence are usually separated by a region of partial non-homology, and it is this region of non-homology that remains deposited in a chromosome of the “Campbell out” cell.

For practicality, in C. glutamicum, typical first and second homologous DNA sequence are at least about 200 base pairs in length, and can be up to several thousand base pairs in length, however, the procedure can be made to work with shorter or longer sequences. For example, a length for the first and second homologous sequences can range from about 500 to 2000 bases, and the obtaining of a “Campbell out” from a “Campbell in” is facilitated by arranging the first and second homologous sequences to be approximately the same length, preferably with a difference of less than 200 base pairs and most preferably with the shorter of the two being at least 70% of the length of the longer in base pairs.

The present invention is further illustrated by the following examples which should not be construed as limiting. The contents of all references, patents and published patent applications cited throughout this application are incorporated herein by reference.

### EXAMPLES

### Example 1

### Generation of the M2014 Strain

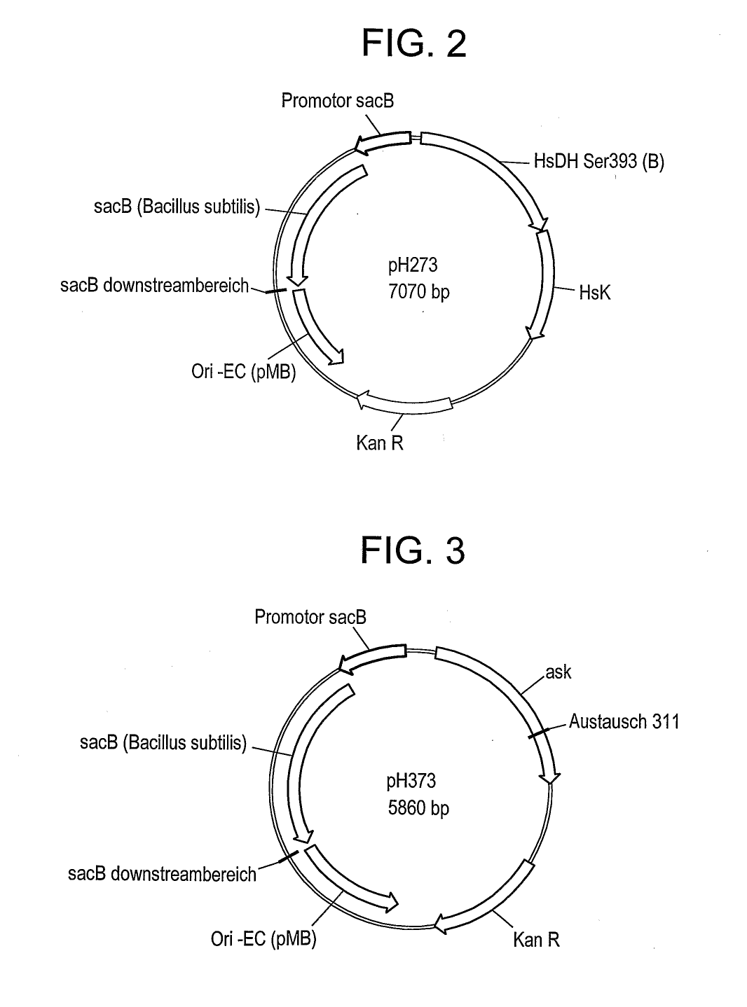

C. glutamicum strain ATCC 13032 was transformed with DNA A (also referred to as pH273) (SEQ ID NO:1) and “Campbelled in” to yield a “Campbell in” strain. FIG. 2 shows a schematic of plasmid pH273. The “Campbell in” strain was then “Campbelled out” to yield a “Campbell out” strain, M440, which contains a gene encoding a feedback resistant homoserine dehydrogenase enzyme (hom^{fbr}). The resultant homoserine dehydrogenase protein included an amino acid change where S393 was changed to F393 (referred to as Hsdh S393F).

The strain M440 was subsequently transformed with DNA B (also referred to as pH373) (SEQ ID NO:2) to yield a “Campbell in” strain. FIG. 3 depicts a schematic of plasmid pH373. The “Campbell in” strain were then “Campbelled out” to yield a “Campbell out” strain, M603, which contains a gene encoding a feedback resistant aspartate kinase enzyme (Ask^{fbr}) (encoded by lysC). In the resulting aspartate kinase protein, T311 was changed to 1311 (referred to as LysC T311I).

It was found that the strain M603 produced about 17.4 mM lysine, while the ATCC13032 strain produced no measurable amount of lysine. Additionally, the M603 strain produced about 0.5 mM homoserine, compared to no measurable amount produced by the ATCC13032 strain, as summarized in Table III.

TABLE III
Amounts of homoserine, O-acetylhomoserine, methionine
and lysine produced by strains ATCC13032 and M603

| | | O-acetyl | | |
|---|---|---|---|---|
| | Homoserine | homoserine | Methionine | Lysine |
| Strain | (mM) | (mM) | (mM) | (mM) |
| ATCC13032 | 0.0 | 0.4 | 0.0 | 0.0 |
| M603 | 0.5 | 0.7 | 0.0 | 17.4 |

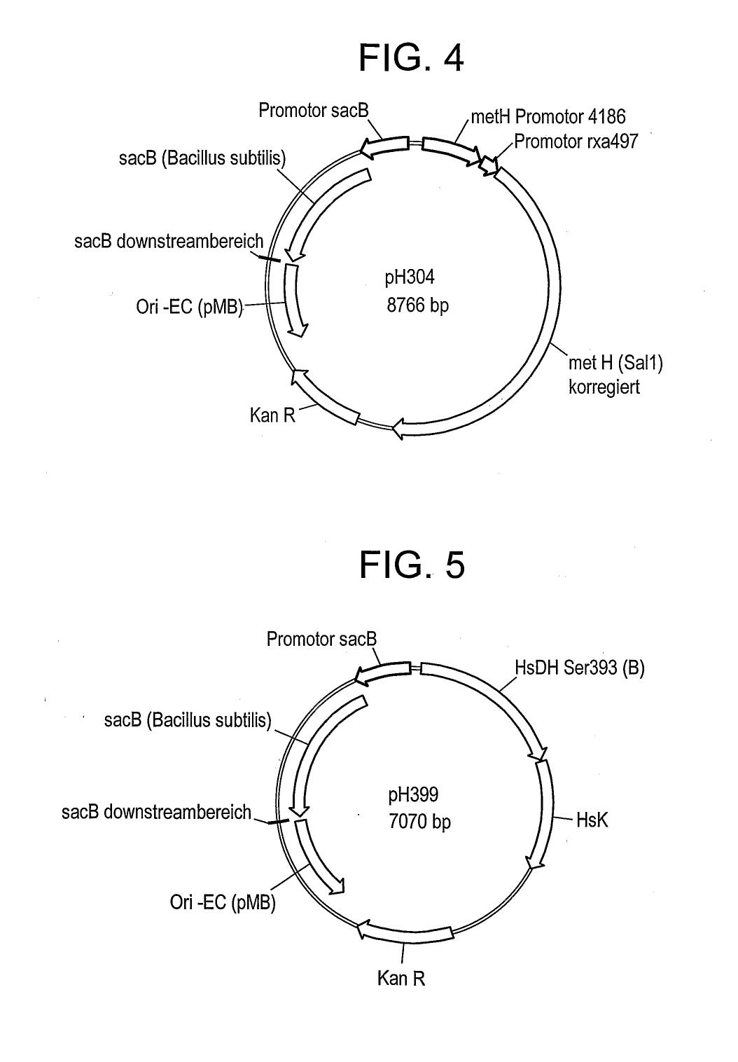

The strain M603 was transformed with DNA C (also referred to as pH304, a schematic of which is depicted in FIG. 4) (SEQ ID NO:3) to yield a “Campbell in” strain, which was then “Campbelled out” to yield a “Campbell out” strain, M690. The M690 strain contained a PgroES promoter upstream of the meth gene (referred to as P₄₉₇ metH). The sequence of the P₄₉₇ promoter is depicted in SEQ ID NO:4. The M690 strain produced about 77.2 mM lysine and about 41.6 mM homoserine, as shown below in Table IV.

TABLE IV
Amounts of homoserine, O-acetyl homoserine, methionine
and lysine produced by the strains M603 and M690

| | | O-acetyl | | |
|---|---|---|---|---|
| | Homoserine | homoserine | Methionine | Lysine |
| Strain | (mM) | (mM) | (mM) | (mM) |
| M603 | 0.5 | 0.7 | 0.0 | 17.4 |
| M690 | 41.6 | 0.0 | 0.0 | 77.2 |

The M690 strain was subsequently mutagenized as follows: an overnight culture of M603, grown in BHI medium (BECTON DICKINSON), was washed in 50 mM citrate buffer pH 5.5, treated for 20 min at 30° C. with N-methyl-N-nitrosoguanidine (10 mg/ml in 50 mM citrate pH 5.5). After treatment, the cells were again washed in 50 mM citrate buffer pH 5.5 and plated on a medium containing the following ingredients: (all mentioned amounts are calculated for 500 ml medium) 10 g (H₄)₂SO₄; 0.5 g KH₂PO₄; 0.5 g K₂HPO₄; 0.125 g MgSO₄.7H₂O; 21 g MOPS; 50 mg CaCl₂; 15 mg protocatechuic acid; 0.5 mg biotin; 1 mg thiamine; and 5 g/l D,L-ethionine (SIGMA CHEMICALS, CATALOG #E5139), adjusted to pH 7.0 with KOH. In addition the medium contained 0.5 ml of a trace metal solution composed of: 10 g/l FeSO₄*7H₂O; 1 g/l MnSO₄*H₂O; 0.1 g/l ZnSO₄*7H₂O; 0.02 g/l CuSO₄; and 0.002 g/l NiCl₂*6H₂O, all dissolved in 0.1 M HCl. The final medium was sterilized by filtration and to the medium, 40 mls of sterile 50% glucose solution (40 ml) and sterile agar to a final concentration of 1.5% were added. The final agar containing medium was poured to agar plates and was labeled as minimal-ethionine medium. The mutagenized strains were spread on the plates (minimal-ethionine) and incubated for 3-7 days at 30° C. Clones that grew on the medium were isolated and restreaked on the same minimal-ethionine medium. Several clones were selected for methionine production analysis.

Methionine production was analyzed as follows. Strains were grown on CM-agar medium for two days at 30° C., which contained: 10 g/l D-glucose, 2.5 g/l NaCl; 2 g/l urea; 10 g/l Bacto Peptone (DIFCO); 5 g/l Yeast Extract (DIFCO); 5 g/l Beef Extract (DIFCO); 22 g/l Agar (DIFCO); and which was autoclaved for 20 min at about 121° C.

After the strains were grown, cells were scraped off and resuspended in 0.15 M NaCl. For the main culture, a suspension of scraped cells was added at a starting OD of 600 nm to about 1.5 to 10 ml of Medium II (see below) together with 0.5 g solid and autoclaved CaCO₃ (RIEDEL DE HAEN) and the cells were incubated in a 100 ml shake flask without baffles for 72 h on a orbital shaking platform at about 200 rpm at 30° C. Medium II contained: 40 g/l sucrose; 60 g/l total sugar from molasses (calculated for the sugar content); 10 g/l (NH₄)₂SO₄; 0.4 g/l MgSO₄*7H₂O; 0.6 g/l KH₂PO₄; 0.3 mg/l thiamine*HCl; 1 mg/l biotin; 2 mg/l FeSO₄; and 2 mg/l MnSO₄. The medium was adjusted to pH 7.8 with NH₄OH and autoclaved at about 121° C. for about 20 min). After autoclaving and cooling, vitamin B₁₂ (cyanocobalamine) (SIGMA CHEMICALS) was added from a filter sterile stock solution (200 μg/ml) to a final concentration of 100 μg/l.

Samples were taken from the medium and assayed for amino acid content. Amino acids produced, including methionine, were determined using the Agilent amino acid method on an Agilent 1100 Series LC System HPLC. (AGILENT). A pre-column derivatization of the sample with ortho-pthalaldehyde allowed the quantification of produced amino acids after separation on a Hypersil AA-column (AGILENT).

Clones that showed a methionine titer that was at least twice that in M690 were isolated. One such clone, used in further experiments, was named M1197 and was deposited on May 18, 2005, at the DSMZ strain collection as strain number DSM 17322. Amino acid production by this strain was compared to that by the strain M690, as summarized below in Table V.

TABLE V
Amounts of homoserine, O-acetylhomoserine, methionine
and lysine produced by strains M690 and M1197

| | | O-acetyl- | | |
|---|---|---|---|---|
| | Homoserine | homoserine | Methionine | Lysine |
| Strain | (mM) | (mM) | (mM) | (mM) |
| M690 | 41.6 | 0.0 | 0.0 | 77.2 |
| M1179 | 26.4 | 1.9 | 0.7 | 79.2 |

The strain M1197 was transformed with DNA F (also referred to as pH399, a schematic of which is depicted in FIG. 5) (SEQ ID NO:5) to yield a “Campbell in” strain, which was subsequently “Campbelled out” to yield strain M1494. This strain contains a mutation in the gene for the homoserine kinase, which results in an amino acid change in the resulting homoserine kinase enzyme from T190 to A190 (referred to as HskT190A). Amino acid production by the strain M1494 was compared to the production by strain M1197, as summarized below in Table VI.

TABLE VI
Amounts of homoserine, O-acetylhomoserine, methionine
and lysine produced by strains M1197 and M1494

| | | O-acetyl- | | |
|---|---|---|---|---|
| | Homoserine | homoserine | Methionine | Lysine |
| Strain | (mM) | (mM) | (mM) | (mM) |
| M1197 | 26.4 | 1.9 | 0.7 | 79.2 |
| M1494 | 18.3 | 0.2 | 2.5 | 50.1 |

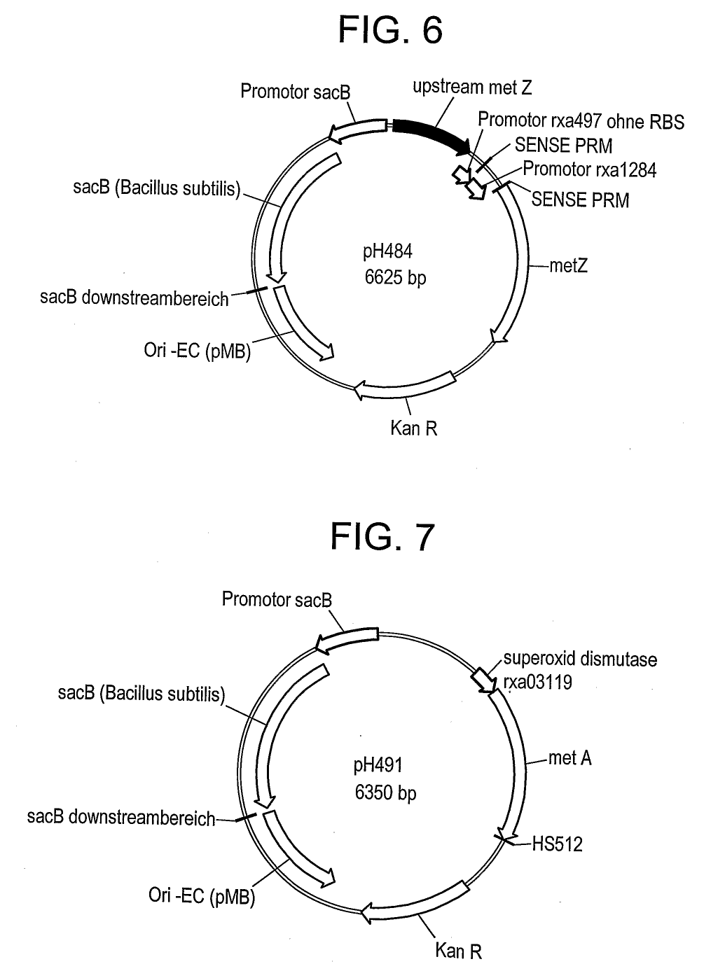

The strain M1494 was transformed with DNA D (also referred to as pH484, a schematic of which is shown in FIG. 6) (SEQ ID NO:6) to yield a “Campbell in” strain, which was subsequently “Campbelled out” to yield the M1990 strain. The M1990 strain overexpresses a metY allele using both a groES-promoter and an EFTU (elongation factor Tu)-promoter (referred to as P₄₉₇ P₁₂₈₄ metY). The sequence of P₄₉₇ P₁₂₈₄ promoter is set forth in SEQ ID NO:7. Amino acid production by the strain M1494 was compared to the production by strain M1990, as summarized below in Table VII.

TABLE VII
Amounts of homoserine, O-acetylhomoserine, methionine
and lysine produced by strains M1494 and M1990

| | | O-acetyl- | | |
|---|---|---|---|---|
| | Homoserine | homoserine | Methionine | Lysine |
| Strain | (mM) | (mM) | (mM) | (mM) |
| M1494 | 18.3 | 0.2 | 2.5 | 50.1 |
| M1990 | 18.2 | 0.3 | 5.6 | 48.9 |

The strain M1990 was transformed with DNA E (also referred to as pH 491, a schematic of which is depicted in FIG. 7) (SEQ ID NO:8) to yield a “Campbell in” strain, which was then “Campbelled out” to yield a “Campbell out” strain M2014: The M2014 strain overexpresses a metA allele using a superoxide dismutase promoter (referred to as P₃₁₁₉ metA). The sequence of P₃₁₁₉ promoter is set forth in SEQ ID NO:9. Amino acid production by the strain M2014 was compared to the production by strain M2014, as summarized below in Table VIII.

TABLE VIII
Amounts of homoserine, O-acetylhomoserine, methionine
and lysine produced by strains M1494 and M1990

| | | O-acetyl- | | |
|---|---|---|---|---|
| | Homoserine | homoserine | Methionine | Lysine |
| Strain | (mM) | (mM) | (mM) | (mM) |
| M1990 | 18.2 | 0.3 | 5.6 | 48.9 |
| M2014 | 12.3 | 1.2 | 5.7 | 49.2 |

### Example 2

### Enhancing the Expression of metF in M2014

Methylenetetrahydrofolate reductase (MetF) catalyzes the reduction of 5,10-methylenetetrahydrofolate to 5-methyltetrahydrofolate (5-MTF)-5-MTF is the methyl donor for the methylation of homocysteine to methionine. Either the MetE or the MetH enzyme catalyzes this methylation. This last step in methionine biosynthesis may be limited if the supply of 5-MTF is sub-optimal. Therefore, the metF gene was modified for constitutive expression. The native promoter of metF was replaced with the groES promoter P₄₉₇) (SEQ ID NO:4) and introduced into the C. glutamicum strain M2014 at the bioAD locus.

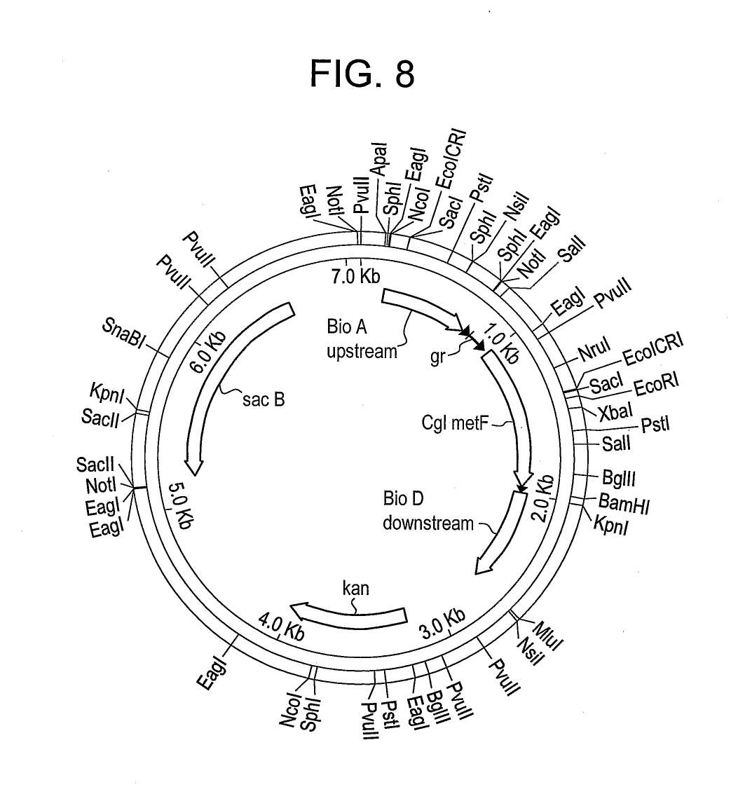

The C. glutamicum metF gene was obtained by PCR and ligated between the XbaI and BamHI sites of the plasmid pOM35, resulting in pOM62 (SEQ ID NO:10). A schematic of the pOM62 plasmid is set forth in FIG. 8. The P₄₉₇ metF cassette was introduced into M2014 at the bioAD chromosomal locus by first selecting for kanamycin resistant transformants (Campbelling in), and then using the sacB counter-selection to isolate kanamycin-sensitive derivatives that had lost the integrating plasmid backbone (Campbelling out). The resulting colonies were screened by PCR to find derivatives of M2014 with the P₄₉₇ metF cassette at the bioAD locus. One such C. glutamicum isolate was called OM41.

To assay for the production of methionine and other amino acids, shake flask cultures were grown in the standard molasses medium as described in Example 3 with strains M2014 in duplicate and strain OM41 in quadruplicate. As shown in Table IX, strain OM41 produced methionine at higher levels than the M2014 strain.

TABLE IX
Amino acids¹ produced by Corynebacterium glutamicum
M2014 and OM41 (a strain containing a P₄₉₇ metF cassette)
in a shake flask experiment at 48 hours.

| Sample | Gly + Hse³ | Met | Lys |
|---|---|---|---|
| OM41 | 1.6 | 1.0 | 6.3 |
| | 1.8 | 1.1 | 6.7 |
| M2014 | 1.7 | 0.9 | 6.6 |
| MM² | 0.06 | 0.05 | 0.0 |

¹Amino acids are measured in g/L. Average of duplicate flasks.
²Molasses Medium.
³Glycine (Gly) and homoserine (Hse) run with the same retention time in the amino acid analysis system used

### Example 3

### Shake Flask Experiments and HPLC Assay

Shake flasks experiments, with the standard Molasses Medium, were performed with strains in duplicate or quadruplicate. Molasses Medium contained in one liter of medium: 40 g glucose; 60 g molasses; 20 g (NH₄)₂ SO₄; 0-4 g MgSO₄*7H₂O; 0.6 g KH₂PO₄; 10 g yeast extract (DIFCO); 5 ml of 400 mM threonine; 2 mgFeSO₄.7H₂O; 2 mg of MnSO₄H₂O; and 50 g CaCO₃ (Riedel-de Haen), with the volume made up with ddH₂O. The pH was adjusted to 7.8 with 20% NH₄OH, 20 ml of continuously stirred medium (in order to keep CaCO₃ suspended) was added to 250 ml baffled Bellco shake flasks and the flasks were autoclaved for 20 min. Subsequent to autoclaving, 4 ml of “4B solution” was added per liter of the base medium (or 80 μl/flask). The “4B solution” contained per liter: 0.25 g of thiamine hydrochloride (vitamin B1), 50 mg of cyancobalamin (vitamin B12), 25 mg biotin, 1.25 g pyridoxine hydrochloride (vitamin B6) and was buffered with 12.5 mM KPO₄, pH 7.0 to dissolve the biotin, and was filter sterilized. Cultures were grown in baffled flasks covered with Biosbield paper secured by rubber bands for 48 hours at 28° C. or 30° C. and at 200 or 300 rpm in a New Brunswick Scientific floor shaker. Samples were taken at 24 hours and/or 48 hours. Cells were removed by centrifugation followed by dilution of the supernatant with an equal volume of 60% acetonitrile and then membrane filtration of the solution using Centricon 0.45 μm spin columns. The filtrates were assayed using HPLC for the concentrations of methionine, glycine plus homoserine, O-acetylhomoserine, threonine, isoleucine, lysine, and other indicated amino acids.

For the HPLC assay, filtered supernatants were diluted 1:100 with 0.45 cm filtered 1 mM Na₂EDTA and 1 μl of the solution was derivatized with OPA reagent (AGILENT) in Borate buffer (80 mM NaBO₃, 2.5 mM EDTA, pH 10.2) and injected onto a 200×4.1 mm Hypersil 5μ AA-ODS column run on an Agilent 1100 series HPLC equipped with a G1321A fluorescence detector (AGILENT). The excitation wavelength was 338 nm and the monitored emission wavelength was 425 nm. Amino acid standard solutions were chromatographed and used to determine the retention times and standard peak areas for the various amino acids. Chem Station, the accompanying software package provided by Agilent, was used for instrument control, data acquisition and data manipulation. The hardware was an HP Pentium 4 computer that supports Microsoft Windows NT 4.0 updated with a Microsoft Service Pack (SP6a).

### Example 4

### Enhancing MetA and MetZ Activity in M2014 and OM41 Increased Methionine Production

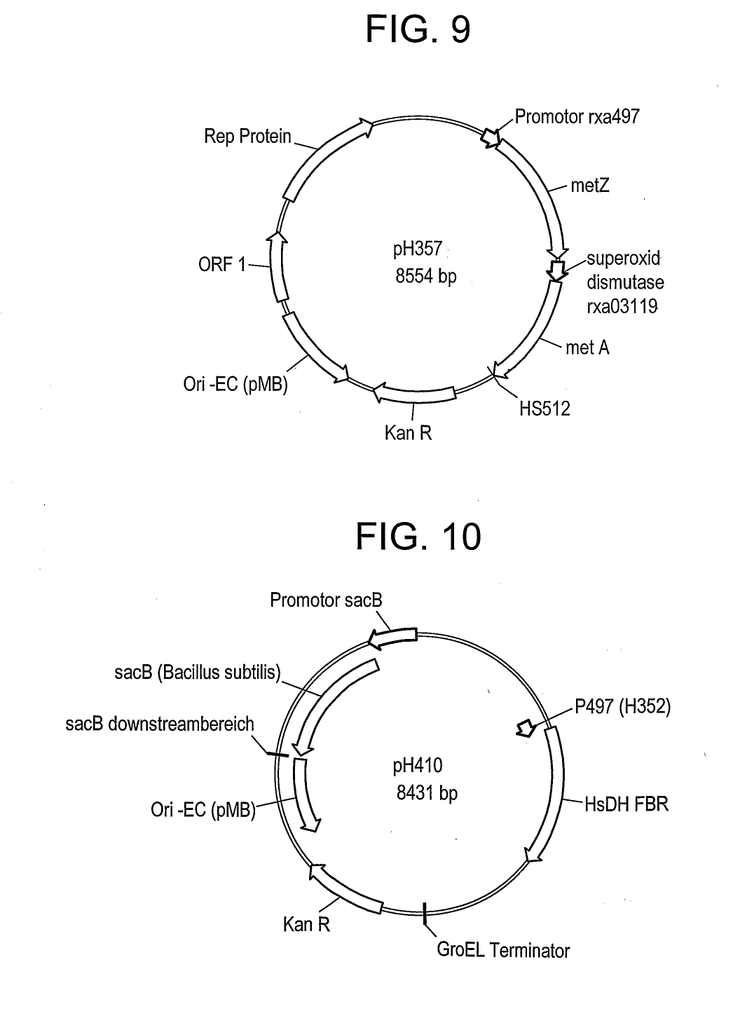

Strains M2014 and OM41 were transformed with the replicating plasmid pH357, a schematic of which is shown in FIG. 9 (SEQ ID:11) containing a P₄₉₇ metZ, P₃₁₁₉ metA cassette. The resulting strains, called M2014(H357) and OM41(H357), were compared to their parent strains in order to determine if additional expression of metZ and/or metA is beneficial for methionine production. In both strains, the presence of the H357 plasmid improved methionine production. As shown in Table X in standard molasses medium, the methionine titer of OM41(H357) was approximately 75% higher than that of OM41, indicating that additional MetA and/or MetZ activity are beneficial for increasing methionine titers (1.4 g/l vs 0.8 g/l). Moreover, the addition of 1% yeast extract (YE) to the medium further increased titers by an additional 30-40%.

TABLE X
48 hour shake flask experiment at 30° C. comparing OM41 to
OM41(H357) in standard molasses medium with or without supplemented
1% yeast extract

| | | Hse + | | | |
|---|---|---|---|---|---|
| | Other | Gly | O-AcHse | Lys | Met |
| Strain | Additions | (g/l) | (g/l) | (g/l) | (mg/l) |
| OM41 | — | 0.9 | 3.7 | 5.1 | 0.8 |
| | — | 0.9 | 3.9 | 5.7 | 0.8 |
| | 1% YE | 1.0 | 1.6 | 4.3 | 1.1 |
| | 1% YE | 1.2 | 1.8 | 4.7 | 1.3 |
| OM41 | — | 1.5 | 3.4 | 3.3 | 1.3 |
| (pH357) | — | 1.6 | 3.8 | 4.0 | 1.5 |
| | 1% YE | 1.8 | 1.2 | 3.1 | 1.9 |
| | 1% YE | 1.8 | 1.3 | 3.5 | 2.0 |
| M2014 | — | 0.2 | 3.4 | 2.2 | 0.4 |
| | — | 0.2 | 3.2 | 2.2 | 0.4 |

### Example 5

### Incorporation of a P₄₉₇ hom^{fbr} Cassette at the pepCK Locus in M2014 Resulted in an Increase in Methionine Production

A feedback resistant homoserine dehydrogenase gene (hom^{fbr}) is present in the chromosome of M2014. This gene, however, uses its native promoter for expression, which is reportedly repressed by methionine. (Rey D. A. et al., J. Molecular Microbiology. 56:871-887 (2005)). In order to obtain a M2014 strain containing a hom gene free from regulation by McbR, a P₄₉₇hom^{fbr} cassette, derived from plasmid pH410, a schematic of which is shown in FIG. 10 (SEQ ID NO:12), was inserted into the pepCK locus of M2014 by Campbelling in and Campbelling out, and subsequently verified by PCR. The resulting strain was called OM224.

Standard shake flask studies were performed on M2014 and OM224, as previously described. As shown in Table XI, OM224 exhibited increased titers of glycine plus homoserine (Gly+Hse), O-acetylhomoserine (O—AcHse), and methionine as compared to M2014; however; there was a decrease in lysine titer as compared to M2014. Amino acids were measured in g/l.

TABLE XI
48 hour shake flask study of the M2014 derivative OM224

| Strain | Cassette | Gly + Hse | O-AcHse | Lys | Met |
|---|---|---|---|---|---|
| M2014 | None | 2.0 | 1.3 | 4.9 | 0.5 |
| | | 2.1 | 1.4 | 5.1 | 0.6 |
| OM224-1 | P₄₉₇ hom^{fbr} | 3.2 | 3.0 | 2.9 | 0.7 |
| | | 2.9 | 2.1 | 2.6 | 0.7 |
| | | 3.1 | 2.6 | 3.4 | 0.8 |
| | | 3.6 | 2.7 | 3.9 | 0.9 |

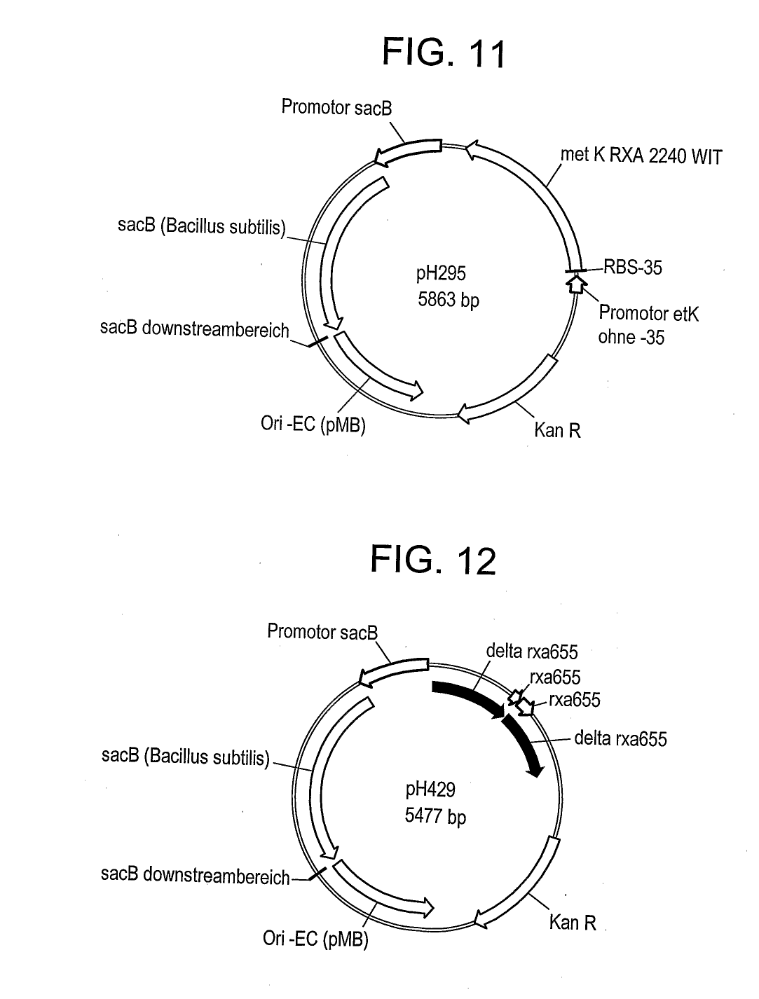

The P₄₉₇ metF cassette was integrated into OM244 strain of the bioAD locus using plasmid pOM62 as described above in Example 2, thereby resulting in strain OM89. OM89 was subsequently modified further by integrating a mutant SAM synthase gene, metK*(C94A) encoding an enzyme with significantly reduced activity compared to the wild-type enzyme (Reczkowski, R. S, and G. D. Markham, J. Biolᵢ Chem., 270:18484-18490 (1995)), at the MetK native-locus. It was expected that lower MetK activity should diminish the production of S-adenosyl methionine. Plasmid pH295 (SEQ ID NO:13), a schematic of which is shown in FIG. 11, was Campbelled in and out of OM89 to replace the wild-type metK in OM89 with metK* resulting in the strain OM99. The metK* allele is identifiable because it introduces a PshAI restriction site into a PCR product derived from the chromosome of OM99. The OM99 strain was next transformed with the replicating plasmid H357, harboring the P₄₉₇ metZ and P₃₁₁₉ meta cassettes, to yield strain OM99(H357).

Standard shake flask experiments were performed on OM89, OM99, OM99(H357), and the parent strains. As shown in Table XII, OM41 and OM224 each produced about 20% more methionine than their parent strain, M2014. OM89 behaved similar to M2014 in this experiment. Integration of the metK* gene into OM89 (strain OM99) appeared to increase methionine titers over the parent strain. Finally, OM99(H357) resulted in a titer of 1.7 g/l methionine, about a 70% increase over the parent strain OM99. All amino acids were measured in g/l.

TABLE XII
Shake flask experiment with various M2014 derivatives

| | | Gly + | | | | |
|---|---|---|---|---|---|---|
| Strain | Cassette | Hse | O-Ac-Hse | Ile | Lys | Met |
| M2014 | | 1.4 | 3.3 | 0.0 | 3.8 | 0.8 |
| | | 1.4 | 3.3 | 0.0 | 3.9 | 0.9 |
| OM41 | P₄₉₇ metF | 0.8 | 4.8 | 0.0 | 4.0 | 1.0 |
| | | 0.9 | 5.9 | 0.0 | 4.6 | 1.1 |
| OM224 | P₄₉₇ hom^{fbr} | 3.6 | 5.2 | 0.1 | 1.8 | 1.0 |
| | | 3.3 | 5.1 | 0.1 | 1.7 | 1.0 |
| OM89 | P₄₉₇ metF | 2.4 | 3.9 | 0.0 | 1.3 | 0.7 |
| | P₄₉₇ hom^{fbr} | 3.5 | 5.3 | 0.1 | 1.7 | 1.0 |
| OM99 | P₄₉₇ metF | 2.7 | 3.4 | 0.1 | 1.6 | 1.0 |
| | P₄₉₇ hom^{fbr} metK* | 3.0 | 3.0 | 0.2 | 1.5 | 1.0 |
| OM99 | P₄₉₇ metF | 1.7 | 1.7 | 0.2 | 0.5 | 1.7 |
| (H357) | P₄₉₇ hom^{fbr} metK* | 1.7 | 1.8 | 0.1 | 0.5 | 1.7 |
| | (H357) | | | | | |

The OM99 (H357) strain also performed well in bench scale fermentations, producing 8.5 g/l of methionine after about 78 hours (see Example 11).

### Example 6

### Deletion of mcbR from M2014 Increased Methionine Production

Plasmid pH429 containing an RXA00655 deletion, (SEQ ID NO: 14), a schematic of which is shown in FIG. 12, was used to introduce the mcbR deletion into C. glutamicum via integration and excision. (See WO 2004/050694 A1). Plasmid pH429 was transformed into the M2014 strain with selection for kanamycin resistance (Campbell in). Using sacB counter-selection, kanamycin-sensitive derivatives of the transformed strain were isolated which presumably had lost the integrated plasmid by excision (Campbell out). The transformed strain produced kanamycin-sensitive derivatives that made small colonies and larger colonies. Colonies of both sizes were screened by PCR to detect the presence of mcbR deletion. None of the larger colonies contained the deletion, whereas 60-70% of the smaller colonies contained the expected mcbR deletion.

When an original isolate was streaked for single colonies on BHI plates, a mixture of tiny and small colonies appeared. When the tiny colonies were restreaked on BHI, once again a mixture of tiny and small colonies appeared. When the small colonies were restreaked on BHI, the colony size was usually small and uniform. Two small single colony isolates, called OM403-4 and OM403-8, were selected for further study.

Shake flask experiments (Table XIII) showed that OM403-8 produced at least twice the amount of methionine as the parent M2014. This strain also produced less than one-fifth the amount of lysine as M2014, suggesting a diversion of the carbon flux from aspartate semialdehyde towards homoserine. A third striking difference was a greater than 10-fold increase in the accumulation of isoleucine by OM403 relative to M2014. Cultures were grown for 48 hours in standard molasses medium.

TABLE XIII
Amino acid production by isolates of the OM403 strain in shake
flask cultures inoculated with freshly grown cells

| | Colony | Deletion | Met | Lys | Hse + Gly | Ile |
|---|---|---|---|---|---|---|
| Strain | size | ΔmcbR | (g/l) | (g/l) | (g/l) | (g/l) |
| M2014 | Large | none | 0.2 | 2.4 | 0.3 | 0.04 |
| | | | 0.2 | 2.5 | 0.3 | 0.03 |
| | | | 0.2 | 2.4 | 0.3 | 0.03 |
| | | | 0.4 | 3.1 | 0.4 | 0.03 |
| OM403-8 | Small | ΔRXA0655 | 1.0 | 0.3 | 0.8 | 0.8 |
| | | | 1.0 | 0.3 | 0.8 | 0.8 |
| | | | 0.9 | 0.3 | 0.8 | 0.8 |
| | | | 1.0 | 0.3 | 0.8 | 0.6 |

Also as shown in Table XIV, there was a greater than 15-fold decrease in the accumulation of O-acetylhomoserine by OM403 relative to M2014. The most likely explanation for this result is that most of the O-acetylhomoserine that accumulates in M2014 is being converted to methionine, homocysteine, and isoleucine in OM403. Cultures were grown for 48 hours in standard molasses medium.

TABLE XIV
Amino acid production by two isolates of OM403 in shake
flask cultures inoculated with freshly grown cells.

| | Deletion | Met | OAc-Hse | Ile |
|---|---|---|---|---|
| Strain | ΔmcbR | (g/l) | (g/l) | (g/l) |
| M2014 | None | 0.4 | 3.4 | 0.1 |
| | | 0.4 | 3.2 | 0.1 |
| OM403-4 | ΔRXA0655 | 1.7 | 0.2 | 0.3 |
| | | 1.5 | 0.1 | 0.3 |
| OM403-8 | ΔRXA0655 | 2.2 | <0.05 | 0.6 |
| | | 2.5 | <0.05 | 0.6 |

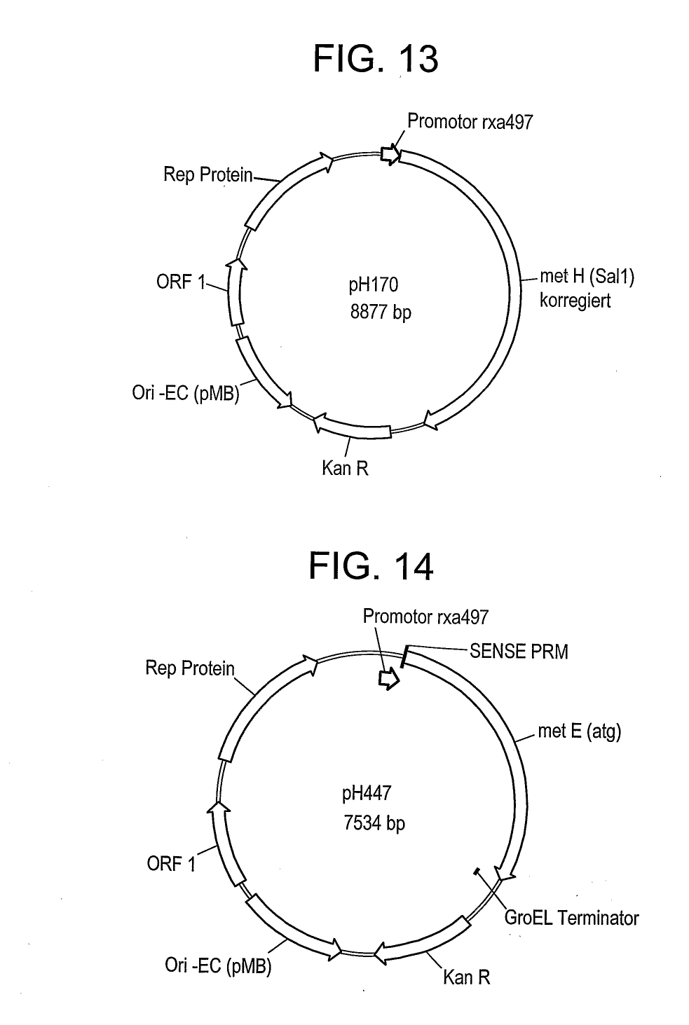

To improve the conversion of homocysteine to methionine in the OM403 background, OM403-8 was transformed with replicating plasmids that cause the overexpression of the meth (pH170) (a schematic of the plasmid pH170 is set forth in FIG. 13 and the sequence in SEQ ID NO: 15) or metE (pH447) (a schematic of the plasmid pH447 is set forth in FIG. 14 and the sequence in SEQ ID NO:16) genes in C. glutamicum. The new strains (OM418 and OM419, respectively) produced more methionine in shale flask experiments than OM403-8 (Table XV).

TABLE XV
Shake flask assays of OM403-8 (M2014 ΔmcbR)
transformed with pH 170 (P₄₉₇ metH), pH 447 (P₄₉₇ metE),
or pH 448 (P₁₂₈₄ metE)

| | | Gly + Hse | OAcHse | Met |
|---|---|---|---|---|
| Strain | plasmid | (g/l) | (g/l) | (g/l) |
| OM403-8 | NONE | 1.2 | 0.4 | 1.5 |
| | | 1.5 | 0.2 | 2.0 |
| OM418-7 | pH 170 | 1.4 | 0.1 | 2.3 |
| -8 | ″ | 1.4 | 0.1 | 2.3 |
| -9 | ″ | 1.3 | 0.1 | 2.1 |
| -10 | ″ | 1.5 | 0.2 | 2.3 |
| -11 | ″ | 1.4 | 0.1 | 2.2 |
| OM403-8 | NONE | 1.1 | 0.3 | 1.7 |
| | ″ | 1.2 | 0.3 | 1.8 |
| OM419-1 | pH 447 | 1.2 | 0.3 | 1.9 |
| -2 | ″ | 1.1 | 0.3 | 1.8 |
| -3 | ″ | 1.5 | 0.3 | 2.4 |
| -4 | ″ | 1.3 | 0.3 | 2.1 |

Cultures were grown for 48 hours in standard molasses medium with or without 25 μg/ml kanamycin. These strains were tested in the fermentor, where OM419 produced significantly more methionine than OM403-8.

### Example 7

### Increasing metF Expression in OM419 Increased Methionine Production

In order to increase metF expression in OM403-8, the native metF promoter was replaced with the E. coli phage lambda P_{R} promoter. This was accomplished using the standard Campbelling in and Campbelling out technique with plasmid pOM427 (SEQ ID NO:17). The resulting strain, called OM428-2, was transformed with the metE expression vector H447. Four isolates of the resulting strain, called OM448, were assayed for methionine production in shake flask assays along with OM403-8 and OM428-2. The results of this experiment, depicted in Table XVI, show that OM428-2 and all four isolates of OM448 produced significantly more methionine than OM403-8, but only one of the four isolates of OM448 produced more methionine than OM428-2.

TABLE XVI
Shake flask assays of OM428-2 and OM448

| | | metF | | | | Lys | Ile | Gly/Hse | OAcHS |
|---|---|---|---|---|---|---|---|---|---|
| Strain | | promoter | plasmid | OD₆₀₀ | Met (g/l) | (g/l) | (g/l) | (g/l) | (g/l) |
| OM403-8 | | Native | none | 31 | 4.1 | 1.4 | 2.7 | 2.7 | 0.3 |
| OM428-2 | | λP_{R} | none | 48 | 5.0 | 1.5 | 3.1 | 3.1 | 0.4 |
| OM448 | -1 | λP_{R} | pH447 | 39 | 5.0 | 1.4 | 3.2 | 3.0 | 0.4 |
| | -2 | λP_{R} | pH447 | 41 | 5.2 | 1.3 | 3.2 | 3.1 | 0.5 |
| | -3 | λP_{R} | pH447 | 42 | 4.7 | 1.2 | 2.8 | 2.9 | 0.7 |
| | -4 | λP_{R} | pH447 | 38 | 4.7 | 1.2 | 3.0 | 2.9 | 0.5 |

### Example 8

### Generation of a Microorganism Containing a Deregulated Sulfate Reduction Pathway

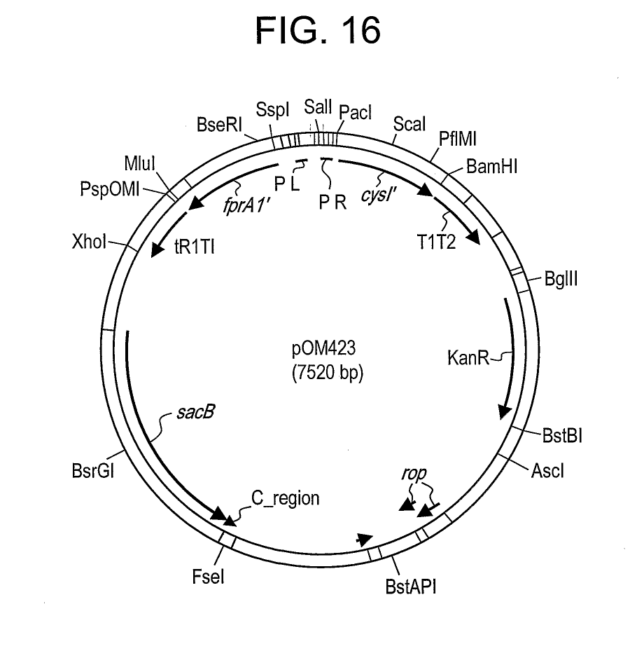

Plasmid pOM423 (SEQ ID NO:18) was used to generate strains that contain a deregulated sulfate reduction pathway. A schematic of the plasmid pOM423 is depicted in FIG. 16. Specifically, an E. coli phage lambda P_{L} and P_{R} divergent promoter construct was used to replace the native sulfate reduction regulon divergent promoters. Strain OM41 was transformed with pOM423 and selected for kanamycin resistance (Campbell in). Following sacB counter-selection, kanamycin sensitive derivatives were isolated from the transformants (Campbell out). These were subsequently analyzed by PCR to determine the promoter structures of the sulfate reduction regulon. Isolates containing the P_{L}-P_{R} divergent promoters were named OM429. Four isolates of OM429 were assayed for sulfate reduction using the DTNB strip test and for methionine production in shake flask assays. To estimate relative sulfide production using the DTNB strip test, a strip of filter paper was soaked in a solution of Ellman's reagent (DTNB) and suspended over a shake flask culture of the strain to be tested for 48 hours. Hydrogen sulfide produced by the growing culture reduces the DTNB, producing a yellow color that is roughly proportional to the amount of H₂S generated. Thus, the intensity of the color produced can be used to obtain a rough estimate of the relative sulfate reduction activity of various strains. The results (Table XVII) show that two of the four isolates displayed relatively high levels of sulfate reduction. These same two isolates also produced the highest levels of methionine. Cultures were grown for 48 hours in standard molasses medium.

TABLE XVII
Methionine production and sulfate reduction
by isolates of OM429 in shake flask cultures

| | Sulfate regulon | Met | DTNB |
|---|---|---|---|
| Strain | promoters | (g/l) | Test |
| M2014 | Native | 1.1 | − |
| OM429-1 | P_{L}/P_{R} | 1.1 | − |
| -2 | | 1.1 | − |
| -3 | | 1.3 | ++ |
| -4 | | 1.4 | ++ |

### Example 9

### Decreasing metQ Expression Decreased Methionine Import

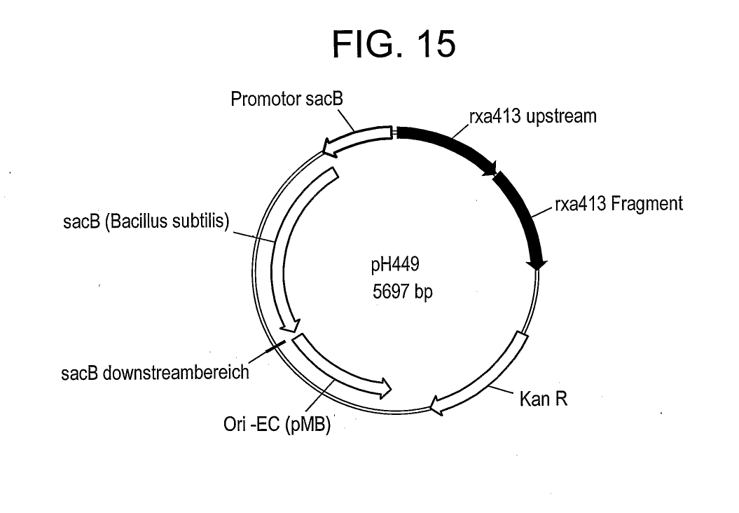

In order to decrease the import of methionine in OM403-8, the promoter and 5′ portion of the metQ gene were deleted. The metQ gene encodes a subunit of a methionine import complex that is required for the complex to function. This was accomplished using the standard Campbelling in and Campbelling out technique with plasmid pH449, a schematic of which is shown in FIG. 15, (SEQ ID NO:19). The resulting strain, called OM456-2, was transformed with the metE expression vector H447 or metF expression plasmid pOM436 (SEQ ID NO:20). Four isolates each of the resulting strains, called OM464 and OM465, respectively, were assayed for methionine production in shake flask assays along with OM403-8 and OM456-2. The results (Table XVIII) show that OM456-2 produced slightly more methionine than OM403-8, and all four isolates of OM464 and OM465 produced more methionine than OM403-8. Cultures were grown for 48 hours in standard molasses medium.

TABLE XVIII

| | | | [Met] | [Lys] | [Gly/Hse] | [OAcHS] | [Ile] |
|---|---|---|---|---|---|---|---|
| Strain | | vector | (g/l) | (g/l) | (g/l) | (g/l) | (g/l) |
| Expt. #1 | | | | | | | |
| OM403-8 | | none | 4.0 | 0.8 | 2.2 | 0.4 | 1.9 |
| | | | 3.9 | 0.6 | 2.2 | 0.4 | 1.9 |
| OM456-2 | | none | 4.2 | 0.4 | 2.3 | 0.4 | 2.3 |
| | | | 4.3 | 0.5 | 2.4 | 0.4 | 2.3 |
| OM464 | -1 | H447 | 4.6 | 1.1 | 2.6 | 0.6 | 2.3 |
| | -2 | ″ | 4.4 | 0.5 | 2.4 | 0.5 | 2.2 |
| | -3 | ″ | 4.3 | 0.5 | 2.3 | 0.5 | 2.1 |
| | -4 | ″ | 4.8 | 0.5 | 2.5 | 0.5 | 2.3 |
| OM465 | -1 | pOM436 | 4.6 | 0.4 | 2.4 | 0.6 | 2.5 |
| | -2 | ″ | 5.2 | 0.6 | 2.8 | 0.4 | 2.9 |
| | -3 | ″ | 4.8 | 0.5 | 2.6 | 0.5 | 2.6 |
| | -4 | ″ | 4.6 | 0.5 | 2.5 | 0.6 | 2.5 |

### Example 10

### Construction of OM469 and OM508

Because deletion of metQ and deregulation of metF each improve methionine production, a strain referred to as OM469, which contains both features, was constructed. OM469 was constructed from strain OM456-2 by replacing the wild type metF promoter with the phage lambda P_{R} promoter. This was accomplished using the standard Campbelling in and Campbelling out technique with plasmid pOM427 (SEQ ID NO:17). Four isolates of OM469 were assayed for methionine production in shake flask culture assays where they all produced more methionine than OM456-2, as shown in Table XIX.

TABLE XIX
Shake flask assays of OM469, a derivative of OM456-2 containing
the phage lambda P_{R} promoter in place of the metF promoter.

| | | metF | | | | [Gly/ | | |
|---|---|---|---|---|---|---|---|---|
| | | pro- | | [Met] | [Lys] | Hse] | [OAcHS] | [Ile] |
| Strain | | moter | metQ | (g/l) | (g/l) | (g/l) | (g/l) | (g/l) |
| OM428-2 | | λP_{R} | native | 4.5 | 0.5 | 2.6 | 0.4 | 2.6 |
| | | | | 4.6 | 0.4 | 2.6 | 0.3 | 2.5 |
| OM456-2 | | native | ΔmetQ | 4.2 | 0.4 | 2.4 | 0.3 | 2.5 |
| | | | | 4.2 | 0.5 | 2.4 | 0.3 | 2.5 |
| OM469 | -1 | λP_{R} | ΔmetQ | 5.0 | 0.5 | 2.7 | 0.4 | 3.1 |
| | -2 | | | 4.9 | 0.5 | 2.7 | 0.4 | 2.8 |
| | -3 | | | 4.8 | 0.4 | 2.6 | 0.4 | 2.7 |
| | -4 | | | 4.7 | 0.5 | 2.6 | 0.4 | 2.8 |

Cultures were grown for 48 hours in standard molasses medium containing 2 mM threonine.

In order to construct strain OM508, strain OM469-2 was transformed with replicating plasmid pH357 (SEQ ID NO: 11). Four isolates of OM508 were assayed for methionine production in shake flask culture assays. Three of the four isolates produced less methionine than OM469 and one of the isolates produced about the same amount of methionine as OM469-2, as depicted in Table XX. All four isolates consumed less glucose than OM469-2, suggesting a higher yield of methionine per mole of glucose.

TABLE XX
Shake flask assays of OM469 containing a metX metY expression
cassette on a replicating vector.

| | | | met genes on | | [Met] | [Lys] | [Gly] | [Hse] | [AHs] | [Ile] |
|---|---|---|---|---|---|---|---|---|---|---|
| Strain | | plasmid | plasmid | Glu* | (g/l) | (g/l) | (g/l) | (g/l) | (g/l) | (g/l) |
| OM469-2 | | pCLIK | none | 0.22 | 4.3 | 0.6 | 2.4 | <0.1 | 0.4 | 1.8 |
| | | | | 0.19 | 3.9 | 0.5 | 2.1 | <0.1 | 0.4 | 1.6 |
| OM508 | -1 | pH357 | X & Y | 17.6 | 3.3 | 0.9 | 1.8 | <0.1 | 0.2 | 0.9 |
| | -2 | | | 20.2 | 3.4 | 0.9 | 1.9 | <0.1 | 0.1 | 0.8 |
| | -3 | | | 18.7 | 3.5 | 1.0 | 1.9 | <0.1 | 0.1 | 0.9 |
| | -4 | | | 23.1 | 4.3 | 1.1 | 2.3 | <0.1 | 0.1 | 1.2 |

Cultures were grown for 48 hours in standard molasses medium containing 2 mM threonine.
*remaining glucose (g/l) at end of 48 hour incubation.

### Example 11

### Fermentation in 7.5 Liter NBS BioFlo 110 Jars

Fed batch fermentations were conducted in 7-liter New Brunswick Scientific (NBS) BioFlo jars with 5-liter working volumes. The sterile batch medium for run M111 included: molasses 150 g/l; glucose 10 g/l; Difco yeast extract 10 g/l; (NH₄)₂SO4 30 g/l; MgSO₄*7H₂O 1 g/l; KH₂PO₄*3H₂O 5 g/l; Mazu DF204C 1.5 g/l (antifoam reagent); 25 mM threonine; 25 mg/l kanamycin; 1×Met Minerals; 1×Met Vitamins; and dH₂0 to 2.0 liters. To this medium was added 150 ml of OM99(H357) inoculum that had been grown for 18 hours at 28° C. in BHI-10 (Becton Dickinson Brain-Heart Infusion medium with 10 g/l glucose added). 1×Met Minerals has a final concentration of 10 mg/l FeSO₄*7H₂O, 10 mg/l MnSO₄*H₂O, 1 mg/l H₃BO₃*4H₂O, 2 mg/l ZnSO₄*7H₂O, 0.25 mg/l CuSO₄, and 0.02 mg/l Na₂MoO₄*2H₂O. 1×Met Vitamins has a final concentration of 6 mg/l nicotinic acid, 9.2 mg/l thiamine, 0.8 mg/l biotin, 0.4 mg/l pyridoxal, and 0.4 mg/l cyancobalamin (vitamin B₁₂), from a 250× filter sterilized stock that contains 12.5 mM potassium phosphate, pH 7.0 to dissolve the biotin.

The fermentation was fed 400 ml of 12.5 mM threonine, plus 12.5 mM isoleucine at a constant rate over a 32 hour period. A separate glucose feed contained glucose 750 g/l, MgSO₄*7H₂O 2 g/l, (NH₄)₂SO₄ 20 g/l, and 10×Met Vitamins in dH₂. The fermentation of OM99 (H357) was fed the glucose and the amino acids feeds separately, but both feeds were begun when the initial glucose level fell to 10 g/l.

The batched initial carbohydrate in the molasses and glucose was consumed during the first 16 to 24 hours after inoculation. After the initial glucose consumption by the cells, glucose concentrations were maintained at between 10 and 15 g/l by feeding the above described glucose solution containing vitamins, magnesium sulfate, and ammonium sulfate.

Agitation was initially set at 200-300 rpm. When the dissolved oxygen concentration falls to 25%, computer control automatically adjusts the agitation rate to maintain a dissolved oxygen concentration of 20±5% [pO₂]. The maximum agitation rate achievable by the hardware was 1200 rpm. When 1200 rpm was not sufficient to maintain a dissolved oxygen level of 20±5% [pO₂], pure oxygen was pulsed into the air supply. The fermentations were maintained at pH 7.0±0.1 and 28°±0.5° C. Computer control and data recording was by New Brunswick Scientific Biocommand software.

Fermentation M111 produced 8.5 g/l methionine in 72 hours and 11.5 g/l methionine in 96 hours. At 96 hours, lysine was 16.5 g/l and O-acetylhomoserine was 8.5 g/l. Therefore, a pool of precursors exists which, if converted to methionine, could increase methionine production an additional 20 g/l.

### Example 12

### Fed Batch Fermentation of OM448-1, Fermentation M190

OM448-1 was fermented as described in Example 11, but starting with the following initial batch medium for run M190: molasses 150 g/l, glucose 10 g/l, Difco yeast extract 20 g/l, (NH₄)₂SO₄ 30 g/l, MgSO₄.7H₂O 1 g/l, KH₂PO₄*3H₂O 12 g/l, HySoyT 20 g/l, Mazu DF204C1.5 g/l, 25 mM threonine, 25 mg/l kanamycin, 1×Met Minerals, 10×Met Vitamins, and dH₂0 to 1.5 liters. To this medium was added 500 ml of OM448-2 inoculum that had been grown for 24 hours at 30° C. in BHySoy-10 (Becton Dickinson Brain-Heart Infusion medium with 10 g/l glucose and 10 g/l HySoy added) to create a starting volume of 2 liters.

The fermentation was fed 400 ml of 30 mM threonine at the rate of 12.5 ml/hr. A separate glucose feed contained glucose 750 g/l, MgSO₄*7H₂O 2 g/l, (NH₄)₂SO₄ 30 g/l, 1×Met Minerals, and 25×Met Vitamins.

Fermentation of OM448-2 in the above described medium produced 16.6 g/1 methionine in 72 hours and 17.1 g/l methionine in 76 hours.

### Example 13

### Fed Batch Fermentation of OM508-4, Fermentation Run M322

OM508-4 was fermented as described in Example 11, but starting with the following initial batch medium for run M322: molasses 150 g/l, Difco yeast extract 20 g/l, (H₄)₂SO₄ 30 g/l, MgSO₄*7H₂O 1 g/l, KH₂PO₄*3H₂O 20 g/l, HySoyT 20 g/l, Mazu DF204C 1.5 g/l, threonine 6 g/l, serine 10 g/l, 25 mg/l kanamycin, 1×Met Minerals, batch Vitamins, and dH₂0 to 1.5 liters. Vitamins were added to the initial batch medium to give a final concentration of 15 mg/l nicotinic acid, 23 mg/l thiamine, 2 mg/l biotin, 1 mg/l pyridoxal, and 1 mg/l cyancobalamin. To 1.5 L of this medium was added 500 ml of OM508-4 inoculum that had been grown for 24 hours at 30° C. in BHySoy-15 (Becton Dickinson Brain-Heart Infusion medium with 15 g/l glucose and 10 g/l HySoy added) to create a starting volume of 2 liters.

The feed contained glucose 750 g/l, MgSO₄*7H₂O 2 g/l, (NH₄)₂SO₄ 40 g/l, serine 10 g/l, threonine 3.6 g/l, 1×Met Minerals and feed Vitamins.

Vitamins were added to the glucose feed to give a final concentration of 75 mg/l nicotinic acid, 115 mg/l thiamine, 10 mg/l biotin, 5 mg/l pyridoxal, and 5 mg/l cyancobalamin in the feed solution. Fermentation of OM508-4 in the above described medium produced 25.8 g/l methionine in 56 hours.

The specification is most thoroughly understood in light of the teachings of the references cited within the specification which are hereby incorporated by reference. The embodiments within the specification provide an illustration of embodiments encompassed by the present invention and should not be construed to limit its scope. The skilled artisan readily recognizes that many other embodiments are encompassed by this invention. All publications and patents cited and sequences identified by accession or database reference numbers described herein are incorporated by reference in their entirety. To the extent the material incorporated by reference contradicts or is inconsistent with the present specification, the present specification will supercede any such material. The citation of any references herein is not an admission that such references are prior art to the present invention.

Unless otherwise indicated, all numbers expressing quantities of ingredients, cell culture, treatment conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained by the present invention. Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the following claims.

What is claimed is:

1. A recombinant microorganism comprising genetic alterations in each of at least five genes chosen from ask^{fbr}, hom^{fbr}, metX, metY, metB, metH, metE, metF and zwf, wherein the genetic alterations lead to overexpression of the at least five genes, thereby resulting in an increased methionine production by the microorganism relative to the methionine produced in absence of the genetic alterations in the at least five genes.

2. A recombinant microorganism comprising genetic alterations in each of at least eight genes chosen from ask^{fbr}, hom^{fbr}, metX, metY, metB, metH, metE, metF and zwf, wherein the genetic alterations lead to overexpression of the at least eight genes, thereby resulting in an increased methionine production by the microorganism relative to the methionine produced in absence of the genetic alterations in the at least eight genes.

3. A recombinant microorganism comprising a combination of:
(a) genetic alterations in each of at least five genes chosen from ask^{fbr}, hom^{fbr}, metX, metY, metB, metH, metE, metF and zwf, thereby resulting in overexpression of each of the at least five genes; and
(b) genetic alterations in at least one gene chosen from mcbR, hsk, metQ, metK and pepCK, thereby resulting in decreased expression of the at least one gene; and
wherein the microorganism produces increased level of methionine relative to the methionine produced in absence of the combination.

4. A recombinant microorganism comprising a combination of:
(a) genetic alterations in each gene chosen from the group consisting of ask^{fbr}, hom^{fbr}, metH and ask^{fbr}, hom^{fbr}, metE, thereby resulting in overexpression of the each gene; and
(b) genetic alterations in each of mcbR and hsk, thereby resulting in decreased expression of mcbR and hsk,
wherein the microorganism produces increased level of methionine relative to the methionine produced in absence of the combination.

5. A recombinant microorganism comprising a combination of:
(a) genetic alterations in each of at least six genes chosen from the group consisting of ask^{fbr}, hom^{fbr}, metX, metY, metF, metH, metE, and ask^{fbr}, hom^{fbr}, metX, metY, metF, metE, thereby resulting in overexpression of each of the at least six genes; and
(b) genetic alterations in each of mcbR and hsk, thereby resulting in decreased expression of mcbR and hsk,
wherein the microorganism produces increased level of methionine relative to the methionine produced in absence of the combination.

6. A recombinant microorganism comprising a combination of:
(a) genetic alterations in each of at least six genes chosen from the group consisting of ask^{fbr}, hom^{fbr} metX, metY, metF, metH and ask^{fbr}, hom^{fbr} metX, metY, metF, metH, metE, thereby resulting in overexpression of each of the at least six genes;
(b) genetic alterations in each of mcbR and hsk, thereby resulting in decreased expression of mcbR and hsk, and
(c) an ethionine-resistant mutation;
wherein the microorganism produces at least 16 g/l methionine under suitable conditions.

7. A recombinant microorganism-comprising genetic alterations in each of at feast eight genes chosen from ask, hom, metX, metY, metB, metH, metE, metF, metC, zwf, frpA, pyc, asd, cysE, cysK, cysM, cysZ, cysC, cysG, cysN, cysD, cysH, cysJ, cysA, cysI, and cysX, wherein the genetic alterations lead to overexpression of the at least eight genes, thereby resulting in increased production of methionine by the microorganism relative to the methionine produced in absence of the genetic alterations.

8. A recombinant microorganism comprising a combination of:
(a) genetic alterations in each of at least five genes chosen from ask, hom, metX, metY, metB, metH, metE, metF, metC, and zwf, wherein the genetic alterations, lead to overexpression of the at least five gene; and
(b) genetic alterations in each of at least six genes chosen from cysM, cysA, cysZ, cysC, cysG, cysJ, cysE, cysK, cysN, cysD, cysH, cysI, and cysX, wherein the genetic alterations lead to overexpression of the at least six genes,
thereby resulting in an increased production of methionine by the microorganism relative to the methionine produced in absence of the combination.

9. A recombinant microorganism comprising a combination of:
(a) genetic alteration in each of at least five genes chosen from ask^{fbr}, hom^{fbr}, metX, metY, metB, metH, metE, metF and zwf, wherein the genetic alterations lead to overexpression of the at least five genes,
(b) genetic alterations in at least one gene chosen from mcbR, hsk, metQ, metK and pepCK, thereby resulting in decreased expression of the at least one gene;
wherein the combination results in a methionine production of at least 8 g/l in under suitable conditions.

10. The recombinant microorganism of any one of claims 1 to 9, wherein the microorganism is Gram positive.

11. The recombinant microorganism of any one of claims 1 to 9, wherein the microorganism is Gram negative.

12. The recombinant microorganism of any one of claims 1 to 9, wherein the microorganism is a microorganism belonging to a genus chosen from Bacillus, Cornyebacterium, Lactobacillus, Lactococci and Streptomyces.

13. The recombinant microorganism of any one of claims 1 to 9, wherein the microorganism belongs to genus Corynebacterium.

14. The recombinant microorganism of claims 13, wherein the microorganism is Corynebacterium glutamicum.

15. A recombinant microorganism chosen from strains M2014, M1119, M1494, M1990, OM41, OM224, OM89, OM99, OM99(H357), OM403, OM418, OM419, OM428, OM429, OM448, OM456, OM464, OM469, OM465, and OM508 or derivatives thereof set forth in claims 1-9.

16. A recombinant microorganism as deposited under DSMZ Accession No. DSM17322.

17. A recombinant microorganism comprising deregulation of at least five proteins chosen from: Aspartate kinase, Homoserine Dehydrogenase, Homoserine Acetyltransferase, Homoserine Succinyltransferase, Cystathionine γ-synthase, Cystathionine β-lyase, O-Acetylhomoserine sulfhydralase, O-Succinylhomoserine sulfhydralase, Vitamin B12-dependent methionine synthase, Vitamin B12-independent methionine synthase, N5,10-methylene-tetrahydrofolate reductase, Sulfate adenylyltrnnsferase subunit 1, Sulfate adenylyltransferase subunit 2, APS kinase, APS reductase, Phosphoadenosine phosphosulfate reductase, NADP-ferredoxin reductase, Sulfite reductase subunit 1, Sulfite reductase subunit 2, Sulfate transporter, Serine O-acetyltransferase, O-acetyl serine (thiol)-lyase A, Uroporphyrinogen III synthase, Glucose-6-phosphate dehydrogenase, Pyruvate carboxylase, and Aspartate semialdehyde dehydrogenase, wherein the deregulation comprises overexpression of the at least five proteins, thereby resulting in production of methionine in an amount of at least 8 g/l under suitable conditions.

18. A method of producing methionine comprising culturing a recombinant microorganism of any of claims 1-5 under conditions such that methionine is produced in an amount of at least 8 g/l.

19. A method of producing methionine comprising:
(a) culturing a Corynebacterium strain comprising genetic alterations in each of at least eight genes chosen from ask, hom, metX, metY, metB, metC, metH, metE, metF, metk, ilvA, metQ, fprA, asd, cysD, cysN, cysC, pyc, cysH, cysI, cysY, cysX, cysZ, cysE, cysK, cysG, zwf, hsk, mcbR and pepCK under conditions such that methionine is produced; and
(b) recovering the methionine.

20. The method of claim 19, wherein the Corynebacterium strain is derived from Corynebacterium glutamicum.

21. The method of claim 19, wherein methionine is produced in an amount of at least 16 g per liter of culture.

22. The method of claim 19, wherein methionine is produced in an amount of at least 25 g/l of culture.
